(19) United States
(12) Patent Application Publication
(10) Pub. No.: US 2010/0158905 A1
(43) Pub. Date: Jun. 24, 2010

(54) COMBINATION THERAPY OF ARTHRITIS WITH TRANILAST

(75) Inventors: Rodney PEARLMAN, El Granada, CA (US); Helen JENKINS, Foster City, CA (US); Tito SERAFINI, Belmont, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: NUON THERAPEUTICS, INC., San Diego, CA (US)

(21) Appl. No.: 12/642,803

(22) Filed: Dec. 19, 2009

Related U.S. Application Data
(60) Provisional application No. 61/139,415, filed on Dec. 19, 2008.

(51) Int. Cl. A61K 31/195; A61K 31/60; A61K 39/395; A61K 38/13; A61K 31/70; A61K 31/4985; A61K 31/56; A61P 19/02

(52) U.S. Cl. 424/133.1; 514/563; 514/165; 514/11; 514/62; 514/249; 514/171

(57) ABSTRACT

Combination therapy is disclosed herein for the treatment an arthritic condition (e.g. rheumatoid arthritis, osteoarthritis or psoriatic arthritis). The therapies disclosed herein comprise administering tranilast or an analogous compound in combination with a pharmaceutical agent, such as a non-steroidal anti-inflammatory drug, a disease-modifying drug, a COX-2 inhibitor, an antibiotic, an analgesic or combination thereof.

### RELATED APPLICATION

This application claims priority to U.S. Provisional Application No. 61/139,415 filed Dec. 19, 2008, the contents of which are hereby incorporated in their entirety by reference.

### FIELD OF THE INVENTION

The present invention relates generally to methods of treating an arthritic condition in a subject in need thereof with tranilast, or derivatives thereof, in combination with another pharmaceutical agent to provide improved therapeutic benefit.

### BACKGROUND

Arthritis (e.g. rheumatoid arthritis, osteoarthritis and psoriatic arthritis) affects more than 20 million Americans and is the leading cause of physical disability and restricted daily activity in more than 7 million Americans. By 2020, this number is expected to grow to more than 60 million Americans.

Rheumatoid arthritis (RA) is considered a chronic, inflammatory autoimmune disorder mainly characterized by inflammation of the lining, or synovium, of the joints. It can lead to long-term joint damage, resulting in chronic pain, loss of function and disability. Rheumatoid arthritis affects about 1% of the U.S. population or 2.1 million Americans. Rheumatoid arthritis is three times more common in women as in men. It afflicts people of all races equally. The disease can begin at any age, but most often starts after age forty and before sixty. In some families, multiple members can be affected, suggesting a genetic basis for the disorder.

Rheumatoid arthritis is generally thought to progress in three stages. The first stage includes swelling of the synovial lining, causing pain, warmth, stiffness, redness and swelling around the joint. The second stage includes rapid division and growth of cells, or pannus, which causes the synovium to thicken. In the third stage, the inflamed cells release enzymes that may digest bone and cartilage, often causing the involved joint to lose its shape and alignment, more pain, and loss of movement. Because rheumatoid arthritis is a systemic disease, it can also affect other organs in the body. Early diagnosis and treatment of rheumatoid arthritis can be critical to continue living a productive lifestyle. Studies have shown that early aggressive treatment of rheumatoid arthritis can limit joint damage, which in turn limits loss of movement, decreased ability to work, higher medical costs and potential surgery.

Osteoarthritis is an acquired musculoskeletal disorder that can occur when the rate of cartilage degradation exceeds that of regeneration, resulting in cartilage erosion, subchondral bone thickening, inflammation and joint damage. Over time, underlying bone can be exposed. The exposed bone is less capable of withstanding mechanical stress and can be prone to microfractures. In addition, localized osteonecrosis can occur beneath the bone surface, leading to cysts that can further weaken the bone's support of the cartilage.

As osteoarthritis progresses, it can eventually influence structures surrounding the joint. Local inflammation such as synovitis can occur, for example in response to inflammatory mediators released during the cartilage degradation process. The joint capsule tends to thicken, and movement of nutrients into and metabolic waste products out of the joint can be restricted. Eventually, periarticular muscle wasting can become evident as osteoarthritis progresses, and the joint is used less often or improperly.

According to the Centers for Disease Control and Prevention (CDC), osteoarthritis is the most common form of arthritic disease. The prevalence of osteoarthritis increases with age, and age is the largest risk factor. A survey reported by Brandt (2001) Principles of Internal Medicine, 15th ed. (Braunwald et al., eds.), New York: McGraw-Hill, pp. 1987-1994, found that only 2% of women less than 45 years old had radiographic evidence of osteoarthritis. In women aged 45 to 64 years, however, the prevalence was 30%, and for those 65 years or older it was 68%. Other risk factors can include excess body weight, genetics, estrogen deficiency, repetitive joint use, and trauma.

Psoriatic arthritis is a chronic inflammatory arthritic condition affecting the skin, the joints, the insertion sites of tendons, ligaments, and fascia. Psoriatic arthritis is commonly associated with psoriasis. Approximately 7% of patients with psoriasis develop psoriatic arthritis. Psoriatic arthritis usually develops in the fourth to sixth decades of life, but it can occur at almost any age.

Psoriatic arthritis may appear in a variety of clinical patterns. There are five general patterns of psoriatic arthritis: arthritis of the distal interphalangeal joints, destructive arthritis, symmetric polyarthritis, asymmetric oligoarthritis, and spondyloarthropathy. Psoriasis appears to precede the onset of psoriatic arthritis in 60-80% of patients. Occasionally, arthritis and psoriasis appear simultaneously. Cutaneous eruptions may be preceded by the arthropathy.

Symptoms of psoriatic arthritis can include extra bone formation, joint stiffness, dactylitis, enthesopathy, tendonitis, and spondylitis. Most patients have the classic psoriasis pattern of skin lesions. Scaly, erythematous plaques; guttate lesions, lakes of pus, and erythroderma are psoriatic skin lesions that may be seen in patients with psoriatic arthritis. Nail lesions, including pitting, Beau lines, leukonychia, onycholysis, oil spots, subungual hyperkeratosis, splinter hemorrhages, spotted lunulae, and cracking, are clinical features significantly associated with the development of psoriatic arthritis. Ocular symptoms in psoriatic arthritis include conjunctivitis, iritis, episcleritis, keratoconjunctivitis sicca and aortic insufficiency.

Although the exact cause of psoriatic arthritis is unknown, genetic, environmental, immunologic, and vascular factors contribute to one's predisposition. The disease is more likely to occur in first-degree relatives who are affected than in the general population. Population studies have shown that multiple human leukocyte antigens (HLA) can be associated with the condition. Much evidence suggests that a T-cell-mediated process drives the pathophysiology of psoriatic arthritis. Activated T cells may contribute to the enhanced production of cytokines found in synovial fluid. Th1 cytokines (e.g., tumor necrosis factor-alpha (TNF-alpha), interleukin (IL)-1-beta and IL-10) can be more prevalent in psoriatic arthritis than in rheumatoid arthritis, suggesting that the two diseases may result from a different mechanism. Monocytes can also play a role in psoriatic arthritis and are responsible for the production of matrix metalloproteinases, which may mediate the destructive changes in the joints of patients with psoriatic arthritis.

The methods disclosed herein offer superior clinical efficacy and long-lasting beneficial results for the treatment of arthritic conditions when compared to the existing treatment approaches.

### SUMMARY OF THE INVENTION

Described herein are combination therapies for the treatment of an arthritic condition (e.g. rheumatoid arthritis) comprising administering to a subject in need thereof tranilast, analogues of tranilast or derivatives thereof, such as compounds of formula I or formula II, and a pharmaceutical agent. The combination therapies disclosed herein can provide a beneficial therapeutic effect, particularly an additive or over-additive effect. As disclosed herein, tranilast, a compound of formula I and/or a compound of formula II can be administered in the same composition containing a pharmaceutical agent; alternately, tranilast, a compound of formula I and/or a compound of formula II can be administered in one composition and the pharmaceutical agent administered in a separate composition. The disclosed compositions can be administered simultaneously or sequentially. One aspect disclosed herein is a method for treating an arthritic condition comprising administering to a subject in need thereof a pharmaceutical composition comprising: a therapeutically effective amount of tranilast or a pharmaceutical acceptable salt thereof, and; a therapeutically effective amount of a pharmaceutical agent. Another aspect disclosed herein is a method for treating an arthritic condition comprising administering to a subject in need thereof a therapeutically effective amount of tranilast or a pharmaceutical acceptable salt thereof, and; a therapeutically effective amount of a pharmaceutical agent. In one embodiment, the pharmaceutical agent is a non-steroidal anti-inflammatory, in another embodiment, the pharmaceutical agent is a DMD; in another embodiment the pharmaceutical agent is etanercept; in yet another embodiment, the pharmaceutical agent is adalimumab; in yet a further embodiment the pharmaceutical agent is a selected COX-2 inhibitor; in another embodiment the pharmaceutical agent is an antibiotic; in yet another embodiment the pharmaceutical agent is an analgesic.

### INCORPORATION BY REFERENCE

All publications and patent applications cited in this specification are herein incorporated by reference in their entirety as if each individual publication or patent application were specifically and individually indicated to be incorporated by reference.

### DETAILED DESCRIPTION OF THE INVENTION

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Described herein are combination therapies for the treatment of an arthritic condition (e.g. rheumatoid arthritis) in a subject in need thereof comprising administering tranilast, analogues of tranilast or derivatives thereof and at least one other pharmaceutical agent. The combination therapies disclosed herein can provide a beneficial therapeutic effect, particularly an additive or over-additive effect. In some embodiments the combination therapies disclosed herein can provide an overall reduction of side effects (e.g. adverse effects). In some embodiments the additive or over-additive beneficial therapeutic effect of the combination therapies disclosed herein provides for dose reduction and/or interval extension when compared to the isolated use of the individual pharmaceutical agents.

An “arthritic condition” herein can be a musculoskeletal disorder, usually accompanied by pain, of one or more joints of a subject. Non limiting examples of arthritic conditions contemplated for treatment herein are rheumatoid arthritis (including juvenile rheumatoid arthritis), osteoarthritis and psoriatic arthritis. Other disorders embraced herein as “arthritic conditions” include, without limitation, infectious arthritis, ankylosing spondylitis, neurogenic arthropathy and polyarthralgia.

### Tranilast and Derivatives

The methods described herein contemplate the use of tranilast in combination with other agents to treat or prevent rheumatoid arthritis. Accordingly, in some embodiments, the methods described herein contemplate the use of tranilast as well as derivatives and compounds generated from modifications of tranilast.

Accordingly, in one embodiment a pharmaceutical composition comprises a compound of formula (A):

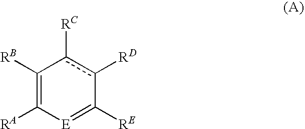

or a pharmaceutically acceptable salt thereof,
wherein
E is selected from N and CR^{N}; [symbol]
represents a single or double bond;
RA is selected from H, C₁₋₄alkyl, OH, C₁₋₄alkoxy, halo, CO₂H and CO₂C₁₋₄alkyl;
R^{B} is selected from H, OH, C₁₋₄alkoxy, halo, or R^{A} and R^{B} together form an optionally substituted fused phenyl or heterocyclic ring;
R^{C} is selected from H, C₁₋₄alkyl, OH, C₁₋₄alkoxy and halo;
R^{D} is selected from H, C₁₋₄alkyl, C₂₋₄alkenyl, OH, C₁₋₄alkoxy, CO₂H, CO₂C₁₋₄alkyl and

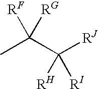

R^{E} is selected from C₁₋₄alkyl, OH, C₁₋₄alkoxy, halo, CO₂H, CO₂C₁₋₄alkyl, NH₂ and NHR^{K};
R^{N} is selected from H, C₁₋₄alkyl, OH and C₁₋₄alkoxy;
R^{F}, R^{G}, R^{H} and R^{I} are each independently H and C₁₋₄alkyl or R^{F} and R^{G} together form an oxo group or R^{F} and R^{H} form a bond;
R^{J} is selected from CH(CO₂H)NH₂, CH(CO₂C₁₋₄alkyl)NH₂, C(O)CO₂H, C(O)CO₂C₁₋₄alkyl, C(O)H, CO₂H, CO₂C₁₋₄alkyl, C(O)NH₂, C(O)NHR^{L}, CH₂NH₂, CH₂NHC₁₋₄alkyl and CH₂N(C₁₋₄alkyl)₂;
R^{K} is selected from H, C₁₋₄alkyl and C(O)H; and
R^{L} is selected from H, C₁₋₄alkyl and optionally substituted phenyl or heterocyclic ring, wherein optionally substituted phenyl or heterocyclic ring is optionally substituted with one or more C₁₋₄alkyl, OH, C₁₋₄alkoxy, CO₂H, tetrazole, CO₂C₁₋₄alkyl, halo, NH₂, NHC₁₋₄alkyl, N(C₁₋₄alkyl)₂,

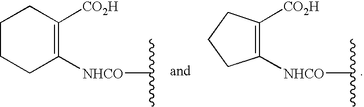

Additional compounds contemplated for use herein are represented by compounds of formula (I):

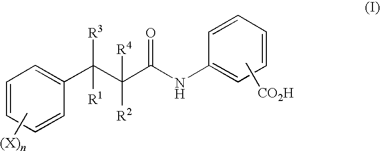

or a pharmaceutically acceptable salt thereof,
wherein
each of R¹ and R² is independently selected from a hydrogen atom or a C₁-C₄ alkyl group,
R³ and R⁴ are each hydrogen atoms or together form another chemical bond,
each X is independently selected from a hydroxyl group, a halogen atom, a C₁-C₄ alkyl group or a C₁-C₄ alkoxy group, or when two X groups are alkyl or alkoxy groups, they may be connected together to form a ring, and
n is an integer from 1 to 3.

The carboxyl group may be in the 2-, 3- or 4-position of the aromatic ring. Generally, the carboxyl group is in the 2-position.

Generally at least one of R¹ and R² is a hydrogen atom. More often, both of R¹ and R² are hydrogen atoms.

Generally R³ and R⁴ taken together form a chemical bond. Such compounds having an unsaturated bond may be in the form of E or Z geometric isomers.

Generally n is 1 or 2 and each X, which may be the same or different, is selected from halogen, C₁-C₄ alkyl or C₁-C₄ alkoxy. Generally X is selected from halogen and C₁-C₄ alkoxy. More often, n is 2 and both X are selected from C₁-C₄ alkoxy, especially when both X are methoxy.

Compounds useful in the invention include those of formula (II):

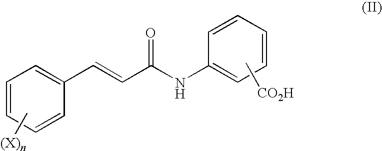

where X and n are defined above.

Examples of compounds of formula (II) include:
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl]-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylpheriyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

One such compound of formula (II) for use in the invention is tranilast (TNL) also known as 2-[[3-(3,4-dimethoxyphenyl)-1-oxo-2-propenyl]amino]benzoic acid. In other embodiments the compound is 3-hydroxykynurenic acid (3-HKA), 3-hydroxyanthranilic acid (3-HAA), picolinic acid (PA), or quinolinic acid (QA).

As used herein, the term “C₁-C₄ alkyl” refers to linear or branched hydrocarbon chains having 1 to 4 carbon atoms. Examples of such groups include methyl, ethyl, n-propyl, isopropyl, n-butyl, sec-butyl and tert-butyl.

As used herein the term “C₂-C₄ alkenyl” refers to linear or branched hydrocarbon chains having 2 to 4 carbon atoms and one or two double bonds. Examples of such groups include vinyl, propenyl, butenyl and butadienyl.

As used herein, the term “C₁-C₄ alkoxy” refers to hydroxy groups substituted with linear or branched alkyl groups having 1 to 4 carbon atoms. Examples of such groups include methoxy, ethoxy, n-propoxy, isopropoxy, n-butoxy, sec-butoxy and tert-butoxy.

As used herein, the term “halogen” or “halo” refers to fluoro, chloro or bromo atoms.

As used herein the term “heterocyclic ring” refers to optionally substituted unsaturated, five- to six-membered cyclic structure in which one or more skeletal atoms is oxygen, nitrogen, sulfur, or combinations thereof. Heterocyclic ring, includes, but is not limited to furanyl, imidazolyl, isoxazolyl, isothiazolyl, oxadiazolyl, oxazolyl, purinyl, pyrazinyl, pyrazolyl, pyridinyl, pyrimidinyl, pyrrolyl, triazolyl, thiazolyl, thiophenyl, tetrazolyl, thiadiazolyl, and thienyl.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Compounds of formula (I) and their pharmaceutically acceptable salts are known and may be prepared by methods known in the art, see U.S. Pat. No. 3,940,422 the contents of which are incorporated herein by reference.

It will also be recognized that some compounds of formula (I) may possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds represented by the present structures, but with the replacement of a hydrogen by a deuterium or tritium, or the replacement of a carbon by a ¹³C- or ¹⁴C-enriched carbon are within the scope of this invention.

In some embodiments, tranilast, a compound of formula (A), a compound of formula (I), or a compound of formula (II) may be modified in order to reduce side effects, improve pharmacokinetic and/or pharmacodynamic profiles. Incorporation of a heavy atom particularly substitution of deuterium for hydrogen, can give rise to an isotope effect that can alter the pharmacokinetics of the drug. The safety profile of a composition may be improved through incorporation of a heavy atom (e.g., deuterium). For example, compositions with substituted deuterium may be delivered in smaller doses with equivalent efficacy. By reducing the dosage, corresponding side effects may be diminished as well.

Replacement within a drug with a heavy isotope can alter its physicochemical properties such as pKa and lipid solubility. These changes may influence the fate of the drug at different steps along its passage through the body. Absorption, distribution, metabolism or excretion can be changed. Absorption and distribution are processes that depend primarily on the molecular size and the lipophilicity of the substance.

Drug metabolism can give rise to large isotopic effect if the breaking of a chemical bond to a deuterium atom is the rate limiting step in the process. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. In any reaction in which the breaking of this bond is the rate limiting step, the reaction will proceed slower for the molecule with the heavy isotope due to kinetic isotope effect. A reaction involving breaking a C—D bond can be up to 700 percent slower than a similar reaction involving breaking a C—H bond.

More caution has to be observed when using deuterium labeled drugs. If the C—D bond is not involved in any of the steps leading to the metabolite, there may not be any effect to alter the behavior of the drug. If a deuterium is placed at a site involved in the metabolism of a drug, an isotope effect will be observed only if breaking of the C—D bond is the rate limiting step. There are evidences to suggest that whenever cleavage of an aliphatic C—H bond occurs, usually by oxidation catalyzed by a mixed-function oxidase, replacement of the hydrogen by deuterium will lead to observable isotope effect. It is also important to understand that the incorporation of deuterium at the site of metabolism slows its rate to the point where another metabolite produced by attack at a carbon atom not substituted by deuterium becomes the major pathway by a process called “metabolic switching.”

For example, substitution of hydrogens with heavier isotopes such as deuterium, i.e., ²H, can afford certain therapeutic advantages resulting from greater metabolic stability, for example increased in vivo half-life or reduced dosage requirements and, hence, may be preferred in some circumstances.

The compounds of formula (I) can be orally active anti-allergic compounds. One such compound of the invention is known by either of the chemical names N-[3,4-dimethoxycinnamoyl]-anthranilic acid or 2-[[3-(3,4-dimethoxyphenyl)-1-oxo-2-propenyl]amino]benzoic acid and may also be referred to as Tranilast. Still further, it is known by the trade name Rizaben. The structure is depicted below:

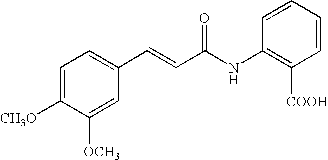

The compounds of formula (A), formula (I) or formula (II) or pharmaceutically acceptable salts thereof or their antagonists may be linked, bound or otherwise associated with any proteinaceous or non-proteinaceous molecules. For example, in one embodiment of the present invention the compounds of formula (I) or pharmaceutically acceptable salts thereof may be associated with a molecule which permits targeting to a localized region.

Metabolites and derivatives of compounds of formula (A), formula (I) and formula (II), including tranilast, and pharmaceutically acceptable salts thereof are contemplated for use herein with another therapy or treatment regime. In some embodiments, the use of tranilast and a second drug or agent can allow the use of a lower dose of the second drug or agent than would ordinarily be used.

The term “mammal” as used herein can include humans, primates, livestock animals (e.g. sheep, pigs, cattle, horses, donkeys), laboratory test animals (e.g. mice, rabbits, rats, guinea pigs), companion animals (e.g. dogs, cats) and captive wild animals (e.g. foxes, kangaroos, deer). The mammal can be a human or a laboratory test animal. In some embodiments the mammal is a human.

The term “subject” as used herein can be a mammal. In some embodiments the term “subject” refers to a human. In some embodiments the human is a human patient.

Reference to “antagonist of a compound of formula (I) or a pharmaceutically acceptable salt thereof' should be understood as a reference to any proteinaceous or non-proteinaceous molecule which directly or indirectly inhibits, retards or otherwise down-regulates the cell functioning inhibitory activity of the compounds of formula (I) or pharmaceutically salts thereof. Identification of antagonists suitable for use in the present invention can be routinely achieved using methods well known to those skilled in the art.

An “effective amount” means an amount necessary at least partly to attain the desired response, or to delay the onset or inhibit progression or halt altogether, the onset or progression of a particular condition being treated. The amount varies depending upon the health and physical condition of the subject to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis can include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Tranilast, compounds of formula (A), compounds of formula (I), compounds of formula (II), pharmaceutically salts thereof and derivatives thereof can be herein referred to as a “modulatory agent” or “modulatory agents.” Modulatory agents and other biologically active agents (e.g. anti-arrythmia agents, anti-hypertension agents, vasodilators, cholesterol or lipid lowering agents and the like) can be referred to herein as agents or active ingredients.

Administration of modulatory agents and all active ingredients disclosed herein, in the form of a pharmaceutical composition, can be performed by any suitable method.

An active ingredient (e.g. a modulatory agent) may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like.

Disclosed herein is a method for treating an arthritic condition in a subject in need thereof comprising administering a therapeutically effective amount of tranilast or a pharmaceutical acceptable salt thereof, and a therapeutically effective amount of a pharmaceutical agent selected from the group consisting of hydroxychloroquine, leflunomide, methotrexate, minocycline, cyclosporine, sulfasalazine, abatacept, adalimumab, entercept, infliximab, rituximab, anakinra, cyclophosphamide, penicillamine, tacrolimus, azathioprine, prednisone, methylprednisolone and pharmaceutically acceptable salts thereof. In one embodiment, the pharmaceutical agent selected from the group consisting of hydroxychloroquine, leflunomide, methotrexate, minocycline, sulfasalazine, abatacept, adalimumab, entercept, infliximab, rituximab, prednisone, methylprednisolone and pharmaceutically acceptable salts thereof. In another embodiment, the pharmaceutical agent selected from the group consisting of hydroxychloroquine, leflunomide, methotrexate, minocycline, sulfasalazine, abatacept, adalimumab, entercept, infliximab, rituximab and pharmaceutically acceptable salts thereof. In one embodiment, the pharmaceutical agent is methotrexate.

Disclosed herein is a method for treating an arthritic condition in a subject in need thereof comprising administering a therapeutically effective amount of tranilast or a pharmaceutical acceptable salt thereof, and a therapeutically effective amount of a non-steroidal anti-inflammatory drug. Also disclosed herein is a method for treating an arthritic condition in a subject in need thereof comprising administering a pharmaceutical composition comprising a therapeutically effective amount of tranilast or a pharmaceutical acceptable salt thereof; and a therapeutically effective amount of a non-steroidal anti-inflammatory drug. In some embodiments the non-steroidal anti-inflammatory drug is selected from the group of ibuprofen, aspirin and naproxen. In some embodiments the arthritic condition is rheumatoid arthritis. In some embodiments the arthritic condition is osteoarthritis. In some embodiments the arthritic condition is psoriatic arthritis.

Disclosed herein is a method for treating an arthritic condition comprising administering to a subject in need thereof a therapeutically effective amount of tranilast or a pharmaceutical acceptable salt thereof, and a therapeutically effective amount of a steroidal anti-inflammatory drug. Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of a steroidal anti-inflammatory drug. In some embodiments the arthritic condition is rheumatoid arthritis. In some embodiments the arthritic condition is osteoarthritis. In some embodiments the arthritic condition is psoriatic arthritis. In some embodiments the steroidal anti-inflammatory drug is selected from the group of alclometasone, amcinonide, betamethasone, betamethasone 17-valerate, clobetasol, clobetasol propionate, clocortolone, cortisone, dehydrotestosterone, deoxycorticosterone, desonide, desoximetasone, dexamethasone, dexamethasone 21-isonicotinate, diflorasone, fluocinonide, fluocinolone, fluorometholone, flurandrenolide, fluticasone, halcinonide, halobetasol, hydrocortisone, hydrocortisone acetate, hydrocortisone cypionate, hydrocortisone hemisuccinate, hydrocortisone 21-lysinate, hydrocortisone sodium succinate, isoflupredone, isoflupredone acetate, methylprednisolone, methylprednisolone acetate, methylprednisolone sodium succinate, methylprednisolone suleptnate, mometasone, prednicarbate, prednisolone, prednisolone acetate, prednisolone hemisuccinate, prednisolone sodium phosphate, prednisolone sodium succinate, prednisolone valerate-acetate, prednisone, triamcinolone, triamcinolone acetonide, and pharmaceutically acceptable salts thereof.

Disclosed herein is a method for treating an arthritic condition comprising administering to a subject in need thereof a therapeutically effective amount of tranilast or a pharmaceutical acceptable salt thereof, and a therapeutically effective amount of a DMD (Disease modifying drug, also known as Disease modifying antirheumatic drug, DMARD) Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of a DMD. In some embodiments the arthritic condition is rheumatoid arthritis. In some embodiments the arthritic condition is osteoarthritis. In some embodiments the arthritic condition is psoriatic arthritis. In some embodiments the DMD is selected from the group consisting of etanercept, adalimumab, infliximab, abatacept, an IL-1 receptor antagonist, a glucocorticoid, penicillamine, hydroxychloroquine sulfate, chlorambucil, cyclosphosphamide, leflunomide, terifluomide, cyclosporine, auranofin, aurothioglucose, azathioprine, gold sodium thiomalate, methotrexate, cyclophosphamide, minocycline, sulfasalazine, rituximab, bucillamine, chloroquine, hydroxychloroquine, 6-mercaptopurine, lobenzarit, misoprostol, glucosamine, tacrolimus and pharmaceutically acceptable salts thereof. In some embodiments the DMD is a TNF antagonist. In some embodiments the TNF antagonist is etanercept, adalimumab or infliximab. In some embodiments, the DMD is abatacept, rituximab, leflunomide, terifluomide, azathioprine, 6-mercaptopurine, chloroquine or hydroxychloroquine. In some embodiments, the DMD is abatacept, rituximab, leflunomide, azathioprine, 6-mercaptopurine, chloroquine or hydroxychloroquine. In some embodiments the DMD is abatacept. In some embodiments the DMD is rituximab. In some embodiments the DMD is leflunomide. In some embodiments the DMD is terifluomide. In some embodiments the DMD is azathioprine. In some embodiments the DMD is 6-mercaptopurine. In some embodiments the DMD is methotrexate. In some embodiments the DMD is chloroquine or hydroxychloroquine. In some embodiments the DMD is a glucocorticoid. In some embodiments the glucocorticoid is budesonide, prednisone or methylprednisolone. In some embodiments the DMD is tacrolimus. In some embodiments the IL-1 receptor antagonist is anakinra. In some embodiments the DMD is a DMOAD (disease-modifying osteoarthritis drug) selected from the group consisting of glucosamine, chondroitin sulfate, calcitonin, alendronate, risedronate, zoledronic acid, teriparatide, VX-765 ((S)-1-((S)-2-{[1-(4-Amino-3-chloro-phenyl)-methanoyl]-amino}-3,3-dimethylbutanoyl)-pyrrolidine-2-carboxylic acid ((2R,3S)-2-ethoxy-5-oxo-tetrahydro-furan-3-yl)-amide, see WO 01/90063), pralnacasan, SB-462795 (relacatib, N-((1S)-3-methyl-1-((((4S,7R)-7-methyl-3-oxo-1-(2-pyridinylsulfony)hexahydro-1H-azepin-4-yl)-amino)carbonyl)-butyl)-1-benzofuran-2-carboxamide), CPA-926 (6-(2-Acetamido-2-deoxy-beta-D-glucopyranosyloxy)-7-hydroxy-2H-1-benzopyran-2-one), ONO-4817 (N-[(1S,3S)-1-[(Ethoxymethoxy)methyl]-4-(hydroxyamino)-3-methyl-4-oxobutyl]-4-phenoxybenzamide), S-3536, PG-530742 (dehydrated salt form of PG-116800), CP-544439 (4-[4-(4-fluorophenoxy)-benzenesulfonylamino]tetrahydropyran-4-carboxylic acid hydroxyamide) and pharmaceutically acceptable salts thereof. In some embodiments the DMOAD is glucosamine or chondroitin sulfate.

Disclosed herein is a method for treating an arthritic condition comprising administering to a subject in need thereof a therapeutically effective amount of tranilast or a pharmaceutical acceptable salt thereof, and a therapeutically effective amount of adalimumab. Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of adalimumab. In some embodiments the arthritic condition is rheumatoid arthritis. In some embodiments the arthritic condition is osteoarthritis. In some embodiments the arthritic condition is psoriatic arthritis.

Disclosed herein is a method for treating an arthritic condition comprising administering to a subject in need thereof a therapeutically effective amount of tranilast or a pharmaceutical acceptable salt thereof, and a therapeutically effective amount of etanercept. Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of etanercept. In some embodiments the arthritic condition is rheumatoid arthritis. In some embodiments the arthritic condition is osteoarthritis. In some embodiments the arthritic condition is psoriatic arthritis.

Disclosed herein is a method for treating an arthritic condition comprising administering to a subject in need thereof a therapeutically effective amount of tranilast or a pharmaceutical acceptable salt thereof, and a therapeutically effective amount of a selective COX-2 inhibitor. Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of a selective COX-2 inhibitor. In some embodiments the arthritic condition is rheumatoid arthritis. In some embodiments the arthritic condition is osteoarthritis. In some embodiments the arthritic condition is psoriatic arthritis. In some embodiments the selective COX-2 inhibitor is selected from the group consisting of celecoxib, deracoxib, valdecoxib, parecoxib, rofecoxib, etoricoxib, lumiracoxib, PAC-10549, cimicoxib, GW-406381, LAS-34475, CS-502 and pharmaceutically acceptable salts thereof. In some embodiments the selective COX-2 inhibitor is celecoxib.

Disclosed herein is a method for treating an arthritic condition comprising administering to a subject in need thereof a therapeutically effective amount of tranilast or a pharmaceutical acceptable salt thereof, and a therapeutically effective amount of an antibiotic. Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of an antibiotic. In some embodiments the arthritic condition is rheumatoid arthritis. In some embodiments the arthritic condition is osteoarthritis. In some embodiments the arthritic condition is psoriatic arthritis. In some embodiments the antibiotic is aminosalicylate, minocycline or doxycycline.

Disclosed herein is a method for treating an arthritic condition comprising administering to a subject in need thereof a therapeutically effective amount of tranilast or a pharmaceutical acceptable salt thereof, and a therapeutically effective amount of an analgesic. Also disclosed herein is a method for treating an arthritic condition comprising administering to a subject in need thereof a pharmaceutical composition comprising a therapeutically effective amount of tranilast or a pharmaceutical acceptable salt thereof, and a therapeutically effective amount of an analgesic. In some embodiments the arthritic condition is rheumatoid arthritis. In some embodiments the arthritic condition is osteoarthritis. In some embodiments the arthritic condition is psoriatic arthritis. In some embodiments the analgesic is diproqualone, lidocaine topical, or an opiate.

Disclosed herein is a method for treating an arthritic condition comprising administering to a subject in need thereof: (a) a therapeutically effective amount of tranilast or a pharmaceutical acceptable salt thereof; and (b) a therapeutically effective amount of a non-steroidal anti-inflammatory drug. In some embodiments the arthritic condition is rheumatoid arthritis. In some embodiments the arthritic condition is osteoarthritis. In some embodiments the arthritic condition is psoriatic arthritis. In one embodiment, the non-steroidal anti-inflammatory drug is ibuprofen, aspirin or naproxen. In another embodiment, the therapeutically effective amount of tranilast or a pharmaceutical acceptable salt thereof and the therapeutically effective amount of an non-steroidal anti-inflammatory drug are comprised in a single pharmaceutical composition.

Disclosed herein is a method for treating an arthritic condition comprising administering to a subject in need thereof: (a) a therapeutically effective amount of tranilast or a pharmaceutical acceptable salt thereof; and (b) a therapeutically effective amount of a DMD selected from the group consisting of etanercept, adalimumab, infliximab, abatacept, an IL-1 receptor antagonist, a glucocorticoid, penicillamine, hydroxychloroquine sulfate, chlorambucil, cyclosphosphamide, leflunomide, cyclosporine, auranofin, aurothioglucose, azathioprine, gold sodium thiomalate, methotrexate, cyclophosphamide, minocycline, sulfasalazine, rituximab, bucillamine, chloroquine, hydroxychloroquine, 6-mercaptopurine, lobenzarit, misoprostol, glucosamine and pharmaceutically acceptable salts thereof. In one embodiment, the therapeutically effective amount of tranilast or a pharmaceutical acceptable salt thereof and the therapeutically effective amount of the DMD are comprised in a single pharmaceutical composition. In some embodiments the arthritic condition is rheumatoid arthritis. In some embodiments the arthritic condition is osteoarthritis. In some embodiments the arthritic condition is psoriatic arthritis. In another embodiment, the DMD is a TNF antagonist selected from the group consisting of etanercept, adalimumab and infliximab. In another embodiment, the DMD is abatacept, rituximab, leflunomide, azathioprine, 6-mercaptopurine, chloroquine or hydroxychloroquine. In yet another embodiment, the DMD is methotrexate. Alternately, the DMD is a glucocorticoid selected from the group consisting of budesonide, prednisone and methylprednisolone. In yet another embodiment, the DMD is a DMOAD selected from the group consisting of glucosamine, chondroitin sulfate, calcitonin, alendronate, risedronate, zoledronic acid, teriparatide, VX-765, pralnacasan, SB-462795, CPA-926, ONO-4817, S 3536, PG-530742, CP-544439 and pharmaceutically acceptable salts thereof.

Disclosed herein is a method for treating an arthritic condition comprising administering to a subject in need thereof: (a) a therapeutically effective amount of tranilast or a pharmaceutical acceptable salt thereof; and (b) a therapeutically effective amount of a selective COX-2 inhibitor, an antibiotic or an analgesic. In one embodiment, the therapeutically effective amount of tranilast or a pharmaceutical acceptable salt thereof and the therapeutically effective amount of the selective COX-2 inhibitor, antibiotic or analgesic are comprised in a single pharmaceutical composition. In some embodiments the arthritic condition is rheumatoid arthritis. In some embodiments the arthritic condition is osteoarthritis. In some embodiments the arthritic condition is psoriatic arthritis. In another embodiment, the selective COX-2 inhibitor is selected from the group consisting of celecoxib, deracoxib, valdecoxib, parecoxib, rofecoxib, etoricoxib, lumiracoxib, PAC-10549, cimicoxib, GW-406381, LAS-34475, CS-502 and pharmaceutically acceptable salts thereof. In another embodiment, the antibiotic is aminosalicylate, minocycline or doxycycline.

In one variation of any aspect or embodiment disclosed herein the arthritic condition is rheumatoid arthritis, osteoarthritis or psoriatic arthritis. In another variation of any aspect or embodiment disclosed herein the arthritic condition is rheumatoid arthritis. In yet another variation of any aspect or embodiment disclosed herein the arthritic condition is osteoarthritis. In one variation of any aspect or embodiment disclosed herein the arthritic condition is psoriatic arthritis.

Disclosed herein is a pharmaceutical composition comprising (a) a therapeutically effective amount of tranilast or a pharmaceutical acceptable salt thereof; and (b) a therapeutically effective amount of a pharmaceutical agent selected from the group consisting of hydroxychloroquine, leflunomide, methotrexate, minocycline, cyclosporine, sulfasalazine, abatacept, adalimumab, entercept, infliximab, rituximab, anakinra, cyclophosphamide, penicillamine, tacrolimus, azathioprine, prednisone, methylprednisolone and pharmaceutically acceptable salts thereof. In one embodiment, the pharmaceutical agent selected from the group consisting of hydroxychloroquine, leflunomide, methotrexate, minocycline, sulfasalazine, abatacept, adalimumab, entercept, infliximab, rituximab, prednisone, methylprednisolone and pharmaceutically acceptable salts thereof. In another embodiment, the pharmaceutical agent selected from the group consisting of hydroxychloroquine, leflunomide, methotrexate, minocycline, sulfasalazine, abatacept, adalimumab, entercept, infliximab, rituximab and pharmaceutically acceptable salts thereof. In one embodiment, the pharmaceutical agent is methotrexate. In one embodiment, the amount of tranilast or salt thereof and the amount of said pharmaceutical agent together are an effective amount to treat rheumatoid arthritis, osteoarthritis or psoriatic arthritis. In one embodiment, the amount of tranilast or salt thereof and the amount of said pharmaceutical agent together are an effective amount to treat rheumatoid arthritis. In another embodiment, the amount of tranilast or salt thereof and the amount of said pharmaceutical agent together are an effective amount to treat osteoarthritis. In another embodiment, the amount of tranilast or salt thereof and the amount of said pharmaceutical agent together are an effective amount to treat psoriatic arthritis.

### Combination Therapies

Pharmaceutical agents (i.e. agents or active ingredients) that are contemplated for use herein in combination with tranilast, a compound of formula (A), a compound of formula (I), a compound of formula (II) or a pharmaceutically acceptable salt thereof include, but are not limited to, an anti-inflammatory agent such as a non-steroidal anti-inflammatory drug (NSAID), a disease-modifying drug (DMD) (e.g. a disease-modifying anti-rheumatic drug (DMARD) or a disease-modifying osteoarthritis drug (DMOAD)), a COX-2 inhibitor, an antibiotic, an analgesic and combinations thereof. Tranilast, a compound of formula (A), a compound of formula (I), a compound of formula (II) or a pharmaceutically acceptable salt thereof can also be combined with a standard treatment algorithm of rheumatoid arthritis, such as disclosed in Saag et al., “American College of Rheumatology 2008 Recommendations for the use of Nonbiologic and Biologic Disease Modifying Antirheumatic Drugs in Rheumatoid Arthritis” Arthritis and Rheumatism (2008) 59(6):762-784. Tranilast, a compound of formula (A), a compound of formula (I), a compound of formula (II) or a pharmaceutically acceptable salt thereof can also be combined with a standard treatment algorithm of rheumatoid arthritis, as reported in “Guidelines for the Management of Rheumatoid Arthritis” Arthritis & Rheumatism (2002) 46(2):328-346, see Table 1.

TABLE 1

| | Approximate time to | |
|---|---|---|
| Drug | benefit | Usual maintenance dose |
| Hydroxychloroquine | 2-6 months | 200 mg twice a day |
| Sulfasalazine | 1-3 months | 1,000 mg 2-3 times a day |
| Methotrexate | 1-2 months | Oral 7.5-20 mg/week; |
| | | injectable 7.5-20 mg/ |
| | | week |
| Leflunomide | 4-12 weeks (skewed earlier) | 20 mg/day in a single dose, |
| | | if tolerated; otherwise, |
| | | 10 mg/day† |
| Etanercept | A few days to 12 weeks | 25 mg subcutaneously twice |
| | | a week |
| Infliximab plus oral and | A few days to 4 months | 3-10 mg IV every 8 weeks |
| subcutaneous methotrexate | | or |
| | | 3-5 mg IV every 4 weeks‡ |
| Azathioprine | 2-3 months | 50-150 mg/day |
| D-penicillamine | 3-6 months | 250-750 mg/day |
| Gold, oral | 4-6 months | 3 mg twice a day |
| Gold, intramuscular | 3-6 months | 25-50 mg intramuscularly |
| | | every 2-4 weeks¶ |
| Minocycline | 1-3 months | 100 mg twice a day |
| Cyclosporine | 2-4 months | 2.5-4 mg/kg/day** |
| Staphylococcal protein A | 3 months | Weekly for 12 weeks |
| immunoadsorption | | |

†The recommended loading dose for leflunomide is 100 mg/day for 3 days.
‡Start infusions at the first visit (week 0), followed by infusions at weeks 2 and 6, and then every 8 weeks thereafter. Can consider increasing the frequency of infusions from every 8 weeks to every 4-6 weeks if there is an incomplete response. IV = intravenous.
¶Start with a 10-mg intramuscular test dose, followed by a loading dose of 50 mg intramuscularly every week until a cumulative dose of 1,000 mg is reached.
**Start at 2.5 mg/kg/day in 2 divided doses taken 12 hours apart, and increase the dosage by 0.5 mg/kg/day every 2-4 weeks until a clinical repsonse is noted or a maximum dosage of 5 mg/kg/day is reached.

Nonlimiting examples of NSAIDs that are contemplated for use herein to treat arthritic conditions include salicylic acid derivatives (such as salicylic acid, acetylsalicylic acid, methyl salicylate, diflunisal, olsalazine, salsalate and sulfasalazine), indole and indene acetic acids (such as indomethacin, etodolac and sulindac), fenamates (such as etofenamic, meclofenamic, mefenamic, flufenamic, niflumic and tolfenamic acids), heteroaryl acetic acids (such as acemetacin, alclofenac, clidanac, diclofenac, fenchlofenac, fentiazac, furofenac, ibufenac, isoxepac, ketorolac, oxipinac, tiopinac, tolmetin, zidometacin and zomepirac), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), enolic acids (such as the oxicam derivatives ampiroxicam, cinnoxicam, droxicam, lornoxicam, meloxicam, piroxicam, sudoxicam and tenoxicam, and the pyrazolone derivatives aminopyrine, antipyrine, apazone, dipyrone, oxyphenbutazone and phenylbutazone), alkanones (such as nabumetone), nimesulide, proquazone, MX-1094, licofelone, and pharmaceutically acceptable salts thereof, and combinations thereof. In some embodiments a method for treating an arthritic condition (e.g. rheumatoid arthritis) comprises administering to a subject in need thereof a pharmaceutical composition comprising a therapeutically effective amount of a compound of formula (A) or a pharmaceutical acceptable salt thereof, and a therapeutically effective amount of an non-steroidal anti-inflammatory drug. In some embodiments a method for treating an arthritic condition (e.g. rheumatoid arthritis) comprises administering to a subject in need thereof a therapeutically effective amount of a compound of formula (A) or a pharmaceutical acceptable salt thereof, and a therapeutically effective amount of an non-steroidal anti-inflammatory drug. In some embodiments a method for treating an arthritic condition (e.g. rheumatoid arthritis) comprises administering to a subject in need thereof a pharmaceutical composition comprising a therapeutically effective amount of a compound of formula (I) and/or a compound of formula (II) or a pharmaceutical acceptable salt thereof, and a therapeutically effective amount of an non-steroidal anti-inflammatory drug. In some embodiments a method for treating an arthritic condition (e.g. rheumatoid arthritis) comprises administering to a subject in need thereof a therapeutically effective amount of a compound of formula (I) and/or a compound of formula (II) or a pharmaceutical acceptable salt thereof, and a therapeutically effective amount of an non-steroidal anti-inflammatory drug. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of an non-steroidal anti-inflammatory drug. In some embodiments a method for treating an arthritic condition comprises administering to a subject in need thereof a therapeutically effective amount of tranilast or a pharmaceutical acceptable salt thereof, and a therapeutically effective amount of a non-steroidal anti-inflammatory drug. In some embodiments the NSAID are selected from the group consisting of salicylic acid derivatives (such as salicylic acid, acetylsalicylic acid, methyl salicylate, diflunisal, olsalazine, salsalate and sulfasalazine), indole and indene acetic acids (such as indomethacin, etodolac and sulindac), fenamates (such as etofenamic, meclofenamic, mefenamic, flufenamic, niflumic and tolfenamic acids), heteroaryl acetic acids (such as acemetacin, alclofenac, clidanac, diclofenac, fenchlofenac, fentiazac, furofenac, ibufenac, isoxepac, ketorolac, oxipinac, tiopinac, tolmetin, zidometacin and zomepirac), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), enolic acids (such as the oxicam derivatives ampiroxicam, cinnoxicam, droxicam, lornoxicam, meloxicam, piroxicam, sudoxicam and tenoxicam, and the pyrazolone derivatives aminopyrine, antipyrine, apazone, dipyrone, oxyphenbutazone and phenylbutazone), alkanones (such as nabumetone), nimesulide, proquazone, MX-1094, licofelone, and pharmaceutically acceptable salts thereof. In some embodiments the NSAID is ibuprofen, aspirin or naproxen.

Nonlimiting examples of DMDs that are contemplated for use herein to treat arthritic conditions include a tumor necrosis factor (TNF) antagonist (e.g. etanercept, adalimumab & infliximab), abatacept, an IL-1 receptor antagonist (e.g. diacerein & anakinra), a glucocorticoid (e.g. budesonide, prednisone, prednisolone, and methylprednisolone), penicillamine, hydroxychloroquine sulfate, chlorambucil, cyclosphosphamide, leflunomide, cyclosporine, auranofin, aurothioglucose, azathioprine, gold sodium thiomalate, methotrexate, cyclophosphamide, minocycline, sulfasalazine, rituximab, bucillamine, chloroquine, hydroxychloroquine, lobenzarit, 6-mercaptopurine, misoprostol, glucosamine, pharmaceutically acceptable salts thereof, and combinations thereof. Therefore, in some embodiments a method for treating an arthritic condition (e.g. rheumatoid arthritis) comprises administering to a subject in need thereof a pharmaceutical composition comprising a therapeutically effective amount of a compound of formula (A) or a pharmaceutical acceptable salt thereof, and a therapeutically effective amount of a DMD. In some embodiments a method for treating an arthritic condition (e.g. rheumatoid arthritis) comprises administering to a subject in need thereof a therapeutically effective amount of a compound of formula (A) or a pharmaceutical acceptable salt thereof, and a therapeutically effective amount of a DMD. In some embodiments a method for treating an arthritic condition (e.g. rheumatoid arthritis) comprises administering to a subject in need thereof a pharmaceutical composition comprising a therapeutically effective amount of a compound of formula (I) and/or a compound of formula (II) or a pharmaceutical acceptable salt thereof, and a therapeutically effective amount of a DMD. In some embodiments a method for treating an arthritic condition (e.g. rheumatoid arthritis) comprises administering to a subject in need thereof a therapeutically effective amount of a compound of formula (I) and/or a compound of formula (II) or a pharmaceutical acceptable salt thereof, and a therapeutically effective amount of a DMD. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of a DMD. In some embodiments a method for treating an arthritic condition comprises administering to a subject in need thereof a therapeutically effective amount of tranilast or a pharmaceutical acceptable salt thereof, and a therapeutically effective amount of a DMD. In some embodiments the DMD is a TNF antagonist. In some embodiments the DMD is etanercept, adalimumab and/or infliximab. In some embodiments the DMD is abatacept. In some embodiments the DMD is selected from the group consisting of an IL-1 receptor antagonist, a glucocorticoid such as prednisone and methylprednisolone, penicillamine, hydroxychloroquine sulfate, chlorambucil, cyclosphosphamide, leflunomide, cyclosporine, auranofin, aurothioglucose, azathioprine, gold sodium thiomalate, methotrexate, cyclophosphamide, minocycline, sulfasalazine, rituximab, bucillamine, chloroquine, hydroxychloroquine, lobenzarit, misoprostol, glucosamine, and pharmaceutically acceptable salts thereof. In some embodiments, the DMD comprises methotrexate in combination with cyclosporine, minocycline, hydroxychloroquine, sulfasalazine, leflunomide or combinations there. In some variations, the DMD comprises sulfasalazine in combination with hydroxychloroquine and methotrexate. In other embodiments the DMD comprises methotrexate in combination with leflunomide. In other embodiments, the DMD comprises cyclosporine in combination with hydroxychloroquine.

Adalimumab, etanercept, and infliximab have demonstrated marked improvements in treating RA when used in combination with methotrexate (Breedveld et al, 2006; Genovese et al, 2005; Keystone et al, 2004; Navarro-Sarabia et al, 2005; Smolen et al, 2006; St. Clair et al, 2004; van der Heijde et al, 2006). Therefore in some embodiments the DMD is methotrexate and a TNF antagonist. In some embodiments, the DMD comprises methotrexate in combination with infliximab; in other embodiments, the DMD comprises methotrexate in combination with etanercept.

In some embodiments the DMD is a DMOAD. Non-limiting examples of DMOADs contemplated for use herein include glucosamine, chondroitin sulfate, calcitonin, alendronate, risedronate, zoledronic acid, teriparatide, VX-765, pralnacasan, SB-462795, CPA-926, ONO-817, S-3536, PG-530742, CP-544439, pharmaceutically acceptable salts thereof, and combinations thereof. Therefore, in some embodiments a method for treating an arthritic condition (e.g. rheumatoid arthritis) comprises administering to a subject in need thereof a pharmaceutical composition comprising a therapeutically effective amount of a compound of formula (A) or a pharmaceutical acceptable salt thereof, and a therapeutically effective amount of a DMOAD. In some embodiments a method for treating an arthritic condition (e.g. rheumatoid arthritis) comprises administering to a subject in need thereof a therapeutically effective amount of a compound of formula (A) or a pharmaceutical acceptable salt thereof, and a therapeutically effective amount of a DMOAD. In some embodiments a method for treating an arthritic condition (e.g. rheumatoid arthritis) comprises administering to a subject in need thereof a pharmaceutical composition comprising a therapeutically effective amount of a compound of formula (I) and/or a compound of formula (II) or a pharmaceutical acceptable salt thereof, and a therapeutically effective amount of a DMOAD. In some embodiments a method for treating an arthritic condition (e.g. rheumatoid arthritis) comprises administering to a subject in need thereof a therapeutically effective amount of a compound of formula (I) and/or a compound of formula (II) or a pharmaceutical acceptable salt thereof, and a therapeutically effective amount of a DMOAD. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of a DMOAD. In some embodiments a method for treating an arthritic condition thereof comprises administering to a subject in need a therapeutically effective amount of tranilast or a pharmaceutical acceptable salt thereof, and a therapeutically effective amount of a DMOAD. In some embodiments the DMOAD is glucosamine or chondroitin.

Nonlimiting examples of COX-2 inhibitor that are contemplated for use herein to treat arthritic conditions include celecoxib, deracoxib, valdecoxib, parecoxib, rofecoxib, etoricoxib, lumiracoxib, PAC-10549, cimicoxib, GW-406381, LAS-34475, CS-502, pharmaceutically acceptable salts thereof, and combinations thereof. Therefore, in some embodiments a method for treating an arthritic condition (e.g. rheumatoid arthritis) comprises administering to a subject in need thereof a pharmaceutical composition comprising a therapeutically effective amount of a compound of formula (A) or a pharmaceutical acceptable salt thereof, and a therapeutically effective amount of a COX-2 selective inhibitor. In some embodiments a method for treating an arthritic condition (e.g. rheumatoid arthritis) comprises administering to a subject in need thereof a therapeutically effective amount of a compound of formula (A) or a pharmaceutical acceptable salt thereof, and a therapeutically effective amount of a COX-2 selective inhibitor. In some embodiments a method for treating an arthritic condition (e.g. rheumatoid arthritis) comprises administering to a subject in need thereof a pharmaceutical composition comprising a therapeutically effective amount of a compound of formula (I) and/or a compound of formula (II) or a pharmaceutical acceptable salt thereof, and a therapeutically effective amount of a COX-2 selective inhibitor. In some embodiments a method for treating an arthritic condition (e.g. rheumatoid arthritis) comprises administering to a subject in need thereof a therapeutically effective amount of a compound of formula (I) and/or a compound of formula (II) or a pharmaceutical acceptable salt thereof, and a therapeutically effective amount of a COX-2 selective inhibitor. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of a COX-2 selective inhibitor. In some embodiments a method for treating an arthritic condition comprises administering to a subject in need thereof a therapeutically effective amount of tranilast or a pharmaceutical acceptable salt thereof, and a therapeutically effective amount of a COX-2 selective inhibitor. In some embodiments the COX-2 selective inhibitor is selected from the group consisting of celecoxib, deracoxib, valdecoxib, parecoxib, rofecoxib, etoricoxib, lumiracoxib, PAC-10549, cimicoxib, GW-406381, LAS-34475, CS-502 and pharmaceutically acceptable salts thereof. In some embodiments the COX-2 selective inhibitor is celecoxib.

Nonlimiting examples of antibiotics that are contemplated for use herein to treat arthritic conditions include aminosalicylate, minocycline and doxycycline. Therefore, in some embodiments a method for treating an arthritic condition (e.g. rheumatoid arthritis) comprises administering to a subject in need thereof a pharmaceutical composition comprising a therapeutically effective amount of a compound of formula (A) or a pharmaceutical acceptable salt thereof, and a therapeutically effective amount of an antibiotic. In some embodiments a method for treating an arthritic condition (e.g. rheumatoid arthritis) comprises administering to a subject in need thereof a therapeutically effective amount of a compound of formula (A) or a pharmaceutical acceptable salt thereof, and a therapeutically effective amount of an antibiotic. In some embodiments a method for treating an arthritic condition (e.g. rheumatoid arthritis) comprises administering to a subject in need thereof a pharmaceutical composition comprising a therapeutically effective amount of a compound of formula (I) and/or a compound of formula (II) or a pharmaceutical acceptable salt thereof, and a therapeutically effective amount of an antibiotic. In some embodiments a method for treating an arthritic condition (e.g. rheumatoid arthritis) comprises administering to a subject in need thereof a therapeutically effective amount of a compound of formula (I) and/or a compound of formula (II) or a pharmaceutical acceptable salt thereof, and a therapeutically effective amount of an antibiotic. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of an antibiotic. In some embodiments a method for treating an arthritic condition comprises administering to a subject in need thereof a therapeutically effective amount of tranilast or a pharmaceutical acceptable salt thereof, and a therapeutically effective amount of an antibiotic. In some embodiments the antibiotic is aminosalicylate, minocycline or doxycycline.

An analgesic can be any member of the diverse group of drugs used to relieve pain (i.e. achieve analgesia). Non-limiting examples of an analgesic contemplated for use herein include an NSAID, a DMD, a COX-2 inhibitor as well as a narcotic (e.g. an opiate or a morphinomimetic). Therefore, in some embodiments a method for treating an arthritic condition (e.g. rheumatoid arthritis) comprises administering to a subject in need thereof a pharmaceutical composition comprising a therapeutically effective amount of a compound of formula (A) or a pharmaceutical acceptable salt thereof, and a therapeutically effective amount of an analgesic. In some embodiments a method for treating an arthritic condition (e.g. rheumatoid arthritis) comprises administering to a subject in need thereof a therapeutically effective amount of a compound of formula (A) or a pharmaceutical acceptable salt thereof, and a therapeutically effective amount of an analgesic. In some embodiments a method for treating an arthritic condition (e.g. rheumatoid arthritis) comprises administering to a subject in need thereof a pharmaceutical composition comprising a therapeutically effective amount of a compound of formula (I) and/or a compound of formula (II) or a pharmaceutical acceptable salt thereof, and a therapeutically effective amount of an analgesic. In some embodiments a method for treating an arthritic condition (e.g. rheumatoid arthritis) comprises administering to a subject in need thereof a therapeutically effective amount of a compound of formula (I) and/or a compound of formula (II) or a pharmaceutical acceptable salt thereof, and a therapeutically effective amount of an analgesic. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of an analgesic. In some embodiments a method for treating an arthritic condition comprises administering to a subject in need thereof a therapeutically effective amount of tranilast or a pharmaceutical acceptable salt thereof, and a therapeutically effective amount of an analgesic. In some embodiments the analgesic is diproqualone, lidocaine topical, or an opiate.

In some embodiments, a method for treating an arthritic condition comprises administering to a subject in need thereof a pharmaceutical composition comprising a therapeutically effective amount of a compound of formula (A), a compound of formula (I) and/or a compound of formula (II) or a pharmaceutical acceptable salt thereof, and a therapeutically effective amount of an agent selected from the group consisting of an NSAID, a DMD (e.g. DMARD or DMOAD), a COX-2 inhibitor, and an antibiotic. In some embodiments, a method for treating an arthritic condition comprises administering to a subject in need thereof a therapeutically effective amount of a compound of formula (A), a compound of formula (I) and/or a compound of formula (II) or a pharmaceutical acceptable salt thereof, and a therapeutically effective amount of an agent selected from the group consisting of an NSAID, a DMD (e.g. DMARD or DMOAD), a COX-2 inhibitor, and an antibiotic. In some embodiments a method for treating an arthritic condition comprises administering to a subject in need thereof a pharmaceutical composition comprising a therapeutically effective amount of tranilast or a pharmaceutical acceptable salt thereof, and a therapeutically effective amount of an agent selected from the group consisting of an NSAID, a DMD (e.g. DMARD or DMOAD), a COX-2 inhibitor, and an antibiotic. In some embodiments a method for treating an arthritic condition comprises administering to a subject in need thereof a therapeutically effective amount of tranilast or a pharmaceutical acceptable salt thereof, and a therapeutically effective amount of an agent selected from the group consisting of an NSAID, a DMD (e.g. DMARD or DMOAD), a COX-2 inhibitor, and an antibiotic. More than one anti-arthritis drug can be administered in combination or adjunctive therapy with a compound of formulas (A), (I) or (II).

Alternate pharmaceutical agents contemplated herein for use in combination with tranilast, a compound of formula (A), a compound of formula (I), a compound of formula (II) or a pharmaceutically acceptable salt thereof include, but are not limited to the agents identified in Table 2. In some embodiments, tranilast, a compound of formula (A), a compound of formula (I), a compound of formula (II) or a pharmaceutically acceptable salt thereof is administered before, concurrently or subsequent to administration of one or more of the compounds listed in Table 2.

TABLE 2
Pharmaceutical agents for combination therapy with tranilast for arthritic conditions

| Development | | Common Drug | Representative Related Basic |
|---|---|---|---|
| Phase | Chemical Name/Description | Name | Patent |
| Clinical | (−)-(3bS,4aS,5aS,6R,6aR,7aS,7bS,8aS, | TPL; PG-490, | U.S. Pat. No. 5,759,550; WO 2004075888; WO |
| | 8bS)-6-Hydroxy-6a-isopropyl-8b- | Triptolide | 2005020887; WO 2006012204; WO |
| | methyl-1,3,3b,4,4a,6,6a,7a,7b,8b,9,10- | | 2006073572 |
| | dodecahydrotrisoxireno[4b,5:6,7:8a,9] | | |
| | phenanthro[1,2-c]furan-1-one | | |
| Clinical | 4-Hydroxy-2-methyl-N-(5- | W-9495, Isoxicam, | U.S. Pat. No. 3,704,298; U.S. Pat. No. 3,787,324 |
| | methylisoxazol-3-yl)-2H-1,2- | Maxicam | |
| | benzothiazine-3-carboxamide 1,1- | | |
| | dioxide | | |
| Clinical | 2-(4′-Chlorobiphenyl-4-ylmethoxy)-2- | ICI-55897, Clozic | |
| | methylpropionic acid | | |
| Clinical | beta,beta-Caroten-3(R)-ol | beta- | JP 2007223914; JP 2007246448; |
| | | Cryptoxanthin; | JP 2008079512; JP 2008208041; |
| | | Cryptoxanthol | JP 2009149635; JP 2009179628; |
| | | | JP 2009184947; JP 2009234925; |
| | | | WO 2004037236; WO 2005112904; |
| | | | WO 2008013219; WO 2008045405; |
| | | | WO 2008067315; WO 2008079287; |
| | | | WO 2008080037; WO 2009084275 |
| Clinical | N-[4(R)-Carbamoyl-1(S)-(3- | CP-481715 | US 2004072834; U.S. Pat. No. 6,673,801; |
| | fluorobenzyl)-2(S),7-dihydroxy-7- | | WO 1998038167; WO 2003000235; |
| | methyloctyl]quinoxaline-2- | | WO 2003000238; WO 2004039375; |
| | carboxamide | | WO 2005115330 |
| Clinical | Water extract from the wood of Taxus | | WO 2004009065 |
| | yunnanensis (Yunnan Hongdoushan) | | |
| IND Filed | | IMO-3100 | |
| Launched | Standarized immunomodulating | OM-89; OM-8980; | |
| | fractions extracted from E. coli, | OM-8915, | |
| | primarily high molecular weight | Subreum | |
| | membrane proteins | | |
| Launched | Purified, native, truncated | CCII; AI-200, | |
| | (telopeptides largely removed) triple | Trinecol (Pullus), | |
| | stranded monomers of type II collagen | Colloral | |
| | from chicken sternal cartilage | | |
| | solubilized in 0.1 M acetic acid. The | | |
| | first 15 residues on the N-terminus of | | |
| | each chain are removed and the last | | |
| | 21, 22 or 24 residues of the C- | | |
| | terminus of each chain are cleaved. | | |
| | Glycosylation can occur on | | |
| | hydroxylysine residues. The substance | | |
| | contains a heterogeneous mixture of | | |
| | alpha-D-glucopyranosyl-(1--2)-beta- | | |
| | D-galactopyranosyl-5-hydroxylysine | | |
| | and beta-D-galactopyranosyl-5- | | |
| | hydroxylysine, and non-glycosylated | | |
| | hydroxylysines. Its molecular weight | | |
| | is approximately 300,000 daltons | | |
| Launched | (3beta,20beta)-3-Hydroxy-11- | NSC-35347; STX- | EP 0565495; EP 1997477; |
| | oxoolean-12-en-29-oic acid | 352; BX-1; GM- | JP 2007137869; JP 2008094793; |
| | | 1658, | JP 2008150294; JP 2008179551; |
| | | Glycyrrhetinic | JP 2009062283; JP 2009062318; |
| | | acid; Glycyrrhetin; | JP 2009062322; JP 2009108023; |
| | | Uralenic acid; | JP 2009149692; JP 2009161462; |
| | | Glycyrrhetic acid; | WO 1990004399; WO 1999059588; |
| | | Enoloxone; 18beta- | WO 2005027882; WO 2007114262; |
| | | Glycyrrhetinic | WO 2008139122; WO 2008152064; |
| | | acid, Arthrodont | WO 2009106963 |
| Launched | 4-Benzamido-5-(dipropylamino)-5- | Proglumetacin | |
| | oxopentanoic acid 3-[4-[2-[2-[1-(4- | maleate, Proxil; | |
| | chlorobenzoyl)-5-methoxy-2-methyl- | Protaxon | |
| | 1H-indol-3- | | |
| | yl]acetoxy]ethyl]piperazin-1-yl]propyl | | |
| | ester dimaleate | | |
| Launched | Purified extract from a mixture of the | SKI-306X, Joins | |
| | oriental herbal medicines Clematis | | |
| | mandshurica (dried root), Prunella | | |
| | vulgaris (dried flower and stem) and | | |
| | Trichosantes kirilowii (dried root) in | | |
| | 1:1:2 (w/w) ratio | | |
| Launched | Combination of misoprostol and | KP-107, | U.S. Pat. No. 5,601,843; U.S. Pat. No. 5,698,225; |
| | diclofenac sodium | Misoprostol/diclofenac | WO 2006099618; WO 2007103540; |
| | | sodium, | WO 2009011967 |
| | | Arthrotec; Artotec | |
| Launched | 2-(2-Hydroxybenzoyloxy)benzoic acid | NSC-49171, | WO 2009006583 |
| | | Salsalate, Salflex; | |
| | | Disalcid | |
| Launched | Standardized ayurvedic herbal | RA-11, Artrex; | U.S. Pat. No. 5,494,668 |
| | preparation containing Withania | Mendar | |
| | somnifera, Boswellia serrata, Zingiber | | |
| | officinale and Curcuma longa | | |
| Launched- | Gold sodium thiomalate | ATM, | WO 2006020994 |
| 1936 | | Aurothiomalate | |
| | | disodium, | |
| | | Tauredon; | |
| | | Aurolate; | |
| | | Myochrysine; | |
| | | Myocrisin | |
| Launched- | 1-Aurothioglucose; (1-Thio-D- | GlcSAu, | WO 2009009523 |
| 1943 | glucopyranosato)gold | Aurothioglucose, | |
| | | Solganal | |
| Launched- | (E)-2-Hydroxy-5-[2-[4-[N-(2- | Sulfasalazine; | DE 2110613; JP 2005325081; |
| 1944 | pyridyl)sulfamoyl]phenyl]diazenyl]benzoic | Salazosulfapyridine; | JP 2007137783; RO 61914; |
| | acid | Sulphasalazine, | US 2008221029; WO 2001072308; |
| | | Salazopyrin; | WO 2004053064; WO 2004082715; |
| | | Salazopyrine; | WO 2004105797; WO 2006003517; |
| | | Azulfidine EN; | WO 2006007312; WO 2006056889; |
| | | Azulfidine | WO 2006125073; WO 2006129386; |
| | | | WO 2007024741; WO 2007055890; |
| | | | WO 2008004231; WO 2008041751; |
| | | | WO 2009129145; WO 2009129149 |
| Launched- | 3-Sulfanyl-D-valine | Penicillamine; D- | WO 2007067331; WO 2008089916; |
| 1953 | | Penicillamine, | WO 2008136000; WO 2009078782 |
| | | Cuprimine; | |
| | | Trolovol; | |
| | | Trisorcin; Pemine; | |
| | | Distamine; | |
| | | Cupramine | |
| Launched- | N-[4-[N-(2,4-Diamino-6- | MPI-5004 | EP 1566202; GB 1595102; |
| 1954 | pteridinylmethyl)-N- | (implant); G-301 | US 2003162695; US 2008193547; |
| | methylamino]benzoyl]-L-glutamic | (topical); APC- | US 2009275529; U.S. Pat. No. 2,512,572; |
| | acid | 2002 (topical); CL- | U.S. Pat. No. 6,544,504; |
| | | 14377, | |
| | | Methotrexate, | |
| | | Intradose-MTX; | |
| | | Rheumatrex; | |
| | | Folex; Metolate; | |
| | | Amethopterin; | |
| | | Maxtrex | |
| Launched- | 17,21-Dihydroxypregnane-1,4-diene- | NSC-10023, | WO 2000072827; WO 2005102271; |
| 1954 | 3,11,20-trione | Deltacortisone; | WO 2005102287; WO 2006008639; |
| | | Deltadehydrocortisone; | WO 2006010394; WO 2006027266; |
| | | Prednisone, | WO 2006035418; WO 2006048736; |
| | | Lodotra; | WO 2006072093; WO 2008015018; |
| | | Deltasone; | WO 2008024484; WO 2009019504; |
| | | Decortin; | WO 2009019505; WO 2009040814; |
| | | Meticorten | WO 2009061587 |
| Launched- | 2-[N-[4-(7-Chloro-4- | Hydroxychloro- | WO 2005027855; WO 2007143174 |
| 1956 | quinolinylamino)pentyl]-N- | quine sulfate, | |
| | ethylamino]-1-ethanol sulfate | Plaquenil | |
| Launched- | 9-Fluoro-11beta,21-dihydroxy- | COL-117, | EP 1782795; GB 1375770; |
| 1958 | 16alpha,17-[(1- | Triamcinolone | US 2009143348; U.S. Pat. No. 2,990,401; |
| | methylethylidene)bis(oxy)]pregnane- | acetonide, | U.S. Pat. No. 6,143,329; WO 1998000178; |
| | 1,4-diene-3,20-dione | Azmacort; | WO 2001082966; WO 2005016352; |
| | | Kenacort; Trinasal; | WO 2005032510; WO 2005046641; |
| | | Nasacort; | WO 2005072701; WO 2005099720; |
| | | Triamonide; | WO 2006003519; WO 2006017347; |
| | | Ledercort; | WO 2006039336; WO 2006043965; |
| | | Adycortyl; | WO 2006055954; WO 2006099591; |
| | | Kenalog; Nasacort | WO 2006102494; WO 2006128735; |
| | | AQ; Trivaris; | WO 2007043365; WO 2008013913; |
| | | AllerNaze; | WO 2008070264; WO 2008119500; |
| | | Triesence; Aftab | WO 2009020561; WO 2009105234; |
| | | | WO 2009114521 |
| Launched- | 6-(1-Methyl-4-nitro-1H-imidazol-5- | AZA-DR; BW-57- | WO 2001007054; WO 2001087324; |
| 1963 | ylsulfanyl)-1H-purine | 322; NSC-39084, | WO 2009020403; WO 2009047001; |
| | | Azathioprine, | WO 2009112849 |
| | | Oraprine; Azanin; | |
| | | Azasan; Imurel; | |
| | | Imurek; Imuran | |
| Launched- | (±)-2-[4-(2- | ST-1482; ZAG- | CA 2156768; DE 3638414; |
| 1969 | Methylpropyl)phenyl]propionic acid | 1701, Ibuprofen, | EP 0250802; EP 0486045; |
| | | CDT-ibuprofen; | EP 0486046; EP 0486964; |
| | | Spedifen; Advil; | EP 0499399; EP 0505180; |
| | | Caldolor; Nurofen | EP 0535841; EP 0546676; |
| | | Meltlets; Brufen; | EP 0560207; EP 1064967; |
| | | Aktren; Advil; | EP 1693051; EP 1721602; |
| | | Amelior; Brufen | EP 1886667; EP 1905428; |
| | | Retard; Motrin; | EP 1920768; EP 1964552; |
| | | Ibuprox; Dolgit; | EP 1974751; EP 2042165; |
| | | Pedea | EP 2110126; EP 553777; |
| Launched- | 5-(Dimethylamino)-9-methyl-2- | DuP-141; AZP; | |
| 1970 | propyl-1H-pyrazolo[1,2- | NSC-102824; MI- | |
| | a][1,2,4]benzotriazine-1,3(2H)-dione | 85; AHR-3018, | |
| | | Apazone; | |
| | | Azapropazone, | |
| | | Rheumox | |
| Launched- | 2(S)-(6-Methoxy-2- | RS-3650, | EP 0587065; EP 1064967; |
| 1973 | naphthyl)propionic acid sodium salt | Naproxen sodium, | EP 1905427; US 2008153888; |
| | | Naprosyn; Synflex; | US 2008181934; US 2008242737; |
| | | Anaprox; Aleve; | US 2008268025; US 2009258843; |
| | | Naproxen; | U.S. Pat. No. 6,451,857; |
| | | Naprelan; Proxen | |
| Launched- | 2-(3-Benzoylphenyl)propionic acid | EN-3269; HKH- | CA 2160739; EP 0679395; |
| 1973 | | 508; HKT-500; | EP 0756870; EP 1688129; EP |
| | | RP-19583; NX- | 1905427; EP 1935405; EP 1972336; |
| | | 304; TQ-1011; | US 2008268025; US 2009130198; |
| | | KPT-220, | U.S. Pat. No. 5,560,924; WO 1993006080; |
| | | Ketoprofen, | WO 1995025511; WO 2000004897; |
| | | Ketocid; Profenid; | WO 2001078721; WO 2001095913; |
| | | Ibifen; Fastum; | WO 2002007767; WO 2004012725; |
| | | Ketoselect; | WO 2004024128; WO 2005023307; |
| | | Ketotransdel; | WO 2005046652; WO 2005063215; |
| | | Nexcede; | WO 2005110482; WO 2005123046; |
| | | Menamin; Mohrus; | WO 2005123136; WO 2005123772; |
| | | ThermoProfen; | WO 2006006799; WO 2006021709; |
| | | Orudis; Ovuvail; | WO 2006050926; WO 2006090833; |
| | | Men-OM; Sector; | WO 2006090839; WO 2006104172; |
| | | Epatec; Miltax; | WO 2006116626; WO 2007049892; |
| | | Keplat; Mohrus | WO 2007109057; WO 2008030359; |
| | | Tape | WO 2008066115; WO 2008092219; |
| | | | WO 2008106220; WO 2008115572; |
| | | | WO 2008150324; WO 2008150995; |
| | | | WO 2009068668 |
| Launched- | 2-(2,6- | XP-21L; HDT-501; | EP 0524582; EP 0595766; |
| 1974 | Dichlorophenylamino)phenylacetic | GP-45840; TDS- | EP 0600395; EP 1064967; |
| | acid sodium salt | 943; DIC-075V; | EP 1550441; EP 1609481; |
| | | TP-318 (gel); | EP 1693051; EP 1930729; |
| | | DCF-100; 318-PW, | EP 1964552; EP 1977734; |
| | | Diclofenac sodium, | EP 2042165; EP 2085077; |
| | | Voltaren lotion; | US 2008206155; US 2008221212; |
| | | Voltaren Tape; | US 2008275122; US 2008286349; |
| | | Voltaren Tape L; | US 2009196907; US 2009220618; |
| | | Pennsaid; Voltaren | U.S. Pat. No. 3,558,690; U.S. Pat. No. 3,778,470; |
| | | gel; Diclofelite CR; | U.S. Pat. No. 4,829,088; U.S. Pat. No. 4,960,799; |
| | | Dyloject; Voltaren; | U.S. Pat. No. 5,603,929; U.S. Pat. No. 5,653,972; |
| | | Dimethaid-D; | U.S. Pat. No. 6,365,184; U.S. Pat. No. 6,488,954; |
| | | Pennsaid Plus; | |
| | | Naboal; Doroxan; | |
| | | Voltaren Ofta; | |
| | | Voltarene; Voltarol | |
| | | Ophtha; Dicloreum | |
| | | CR; Dealgic; | |
| | | DicloFoam; | |
| | | ProSorb-D; | |
| | | Voltarol; Rectos | |
| Launched- | (±)-2-(3-Phenoxyphenyl)propionic | LY-69323, | EP 0221732; EP 1905427; |
| 1974 | acid calcium salt | Fenoprofen | WO 2000004897; WO 2001078721 |
| | | calcium, Fepron; | |
| | | Nalgesic; Nalfon | |
| Launched- | (Z)-5-Fluoro-2-methyl-1-[4- | MK-231, Sulindac, | DE 2039426; EP 1905427; |
| 1976 | (methylsulfinyl)benzylidene]-1H- | Clinoril; Klinoril; | JP 2005247807; US 2003220266; |
| | indene-3-acetic acid | Arthrocine; | U.S. Pat. No. 3,647,858; U.S. Pat. No. 6,051,587; |
| | | Imbaral; Sulindal | WO 2000004897; WO 2001012227; |
| | | | WO 2001035956; WO 2001095913; |
| | | | WO 2003037373; WO 2003061713; |
| | | | WO 2005009354; WO 2005020933; |
| | | | WO 2005054181; WO 2006102439; |
| | | | WO 2009129147; WO 2009129149; |
| | | | WO 2009129510; WO 2009137400 |
| Launched- | 1-Methyl-5-(4-methylbenzoyl)-1H- | McN-2559, | EP 1905427; WO 2006039704; |
| 1976 | pyrrole-2-acetic acid | Tolmetin, Tolectin | WO 2007022356; WO 2009072139 |
| Launched- | 4-(4-Biphenylyl)-4-oxobutyric acid | CL-82204, | EP 1905427; U.S. Pat. No. 3,784,701 |
| 1976 | | Fenbufen, Cinopal; | |
| | | Lederfen | |
| Launched- | (±)-2-Fluoro-alpha-methyl-4- | U-27182; BTS- | EP 0223369; EP 0485111; |
| 1977 | biphenylacetic acid | 18322; MKS-11, | EP 0615447; EP 1905427; |
| | | Anmetarin; | EP 1992333; EP 2074990; |
| | | Flurbiprofen, | EP 2077104; U.S. Pat. No. 3,755,427; |
| | | Adofeed; Antadys; | U.S. Pat. No. 4,188,491; U.S. Pat. No. 4,266,069; |
| | | Ocufen; Ocuflur; | U.S. Pat. No. 5,206,029; U.S. Pat. No. 5,321,017; |
| | | Yakuban; Ansaid; | U.S. Pat. No. 5,556,638; |
| | | Flurofen; Froben; | |
| | | Flugalin; Cebutid | |
| Launched- | 2′,4′-Difluoro-4-hydroxy-1,1′- | MK-647, | EP 0293529; GB 1175212; |
| 1978 | biphenyl-3-carboxylic acid; 2- | Diflunisal, Dolobid | GB 1496231; U.S. Pat. No. 3,674,870; |
| | Hydroxy-5-(2,4- | | U.S. Pat. No. 4,225,730; |
| | difluorophenyl)benzoic acid | | |
| Launched- | 4-Hydroxy-2-methyl-N-(2-pyridyl)- | CP-16171, | CA 2119531; EP 0568026; |
| 1979 | 2H-1,2-benzothiazine-3-carboxamide | Piroxicam, Felden; | U.S. Pat. No. 3,591,584; U.S. Pat. No. 5,362,758; |
| | 1,1-dioxide | Baxo; Feldene Gel; | U.S. Pat. No. 5,436,241; U.S. Pat. No. 5,601,843; |
| | | Feldene Fast; | U.S. Pat. No. 5,698,225; U.S. Pat. No. 6,051,587; |
| | | Feldene | |
| Launched- | 1-(4-Chlorobenzoyl)-5-methoxy-2- | TVX-1322; Bay-f- | WO 2000004897; WO 2004094409; |
| 1980 | methyl-1H-indole-3-acetic acid | 4975, Acemetacin, | WO 2007014476 |
| | carboxymethyl ester; 1-(4- | Emflex; Rantudil | |
| | Chlorobenzoyl)-5-methoxy-2- | | |
| | methylindole-3-acetoxyacetic acid | | |
| Launched- | 2-(2,6-Dichloro-3- | CI-853 (free acid), | U.S. Pat. No. 3,313,848 |
| 1980 | methylphenylamino)benzoic acid | Meclofenamate | |
| | sodium salt hydrate | sodium (free acid), | |
| | | Meclodol; | |
| | | Lenidolor; | |
| | | Meclomen | |
| Launched- | (2,3,4,6-Tetra-O-acetyl-1-thio-beta-D- | SK&F-39162, | U.S. Pat. No. 3,708,579; WO 2009137071 |
| 1982 | glucopyranosato- | Auranofin, | |
| | S)(triethylphosphine)gold; (1-Thio- | Ridaura; Ridauran | |
| | beta-D-glucopyranosato- | | |
| | S)(triethylphosphine)gold 2,3,4,6- | | |
| | tetraacetate | | |
| Launched- | Cyclo[[(E)-(2S,3R,4R)-3-hydroxy-4- | ST-603; DE-076; | EP 0034567; EP 0504760; |
| 1983 | methyl-2-(methylamino)-6-octenoyl]- | OLO-400; Nova- | EP 0539319; EP 1142566; |
| | L-2-aminobutyryl-N-methylglycyl-N- | 22007; Sang-2000; | EP 1762248; JP 2004244347; |
| | methyl-L-leucyl-L-valyl-N-methyl-L- | CB-01-09 MMX; | US 2002018776; US 2003171263; |
| | leucyl-L-alanyl-D-alanyl-N-methyl-L- | CSA; CyA; OL- | US 2006148686; US 2006257326; |
| | leucyl-N-methyl-L-leucyl-N-methyl- | 27400; Sang-35, | US 2007027072; US 2007087962; |
| | L-valyl]; [R-[R*,R*-(E)]]-Cyclic-(L- | Cyclosporin A; | US 2007105761; US 2008146497; |
| | alanyl-D-alanyl-N-methyl-L-leucyl-N- | Cyclosporine A; | US 2008262078; US 2008267977; |
| | methyl-L-leucyl-N-methyl-L-valyl-3- | Cyclosporin; | US 2008299206; US 2009130198; |
| | hydroxy-N,4-dimethyl-L-2-amino-6- | Cyclosporine; | US 2009155929; U.S. Pat. No. 5,382,655; |
| | octenoyl-L-alpha-aminobutyryl-N- | Ciclosporin, | U.S. Pat. No. 6,444,643; U.S. Pat. No. 6,503,883; |
| | methylglycyl-N-methyl-L-leucyl-L- | Mitogard; Vekacia; | U.S. Pat. No. 6,582,718; U.S. Pat. No. 6,696,413; |
| | valyl-N-methyl-L-leucyl) | Consupren S; | U.S. Pat. No. 6,960,563; U.S. Pat. No. 6,979,672; |
| | | Sandimmun; | |
| | | Modusik-A; | |
| | | Equoral; Pulminiq; | |
| | | Papilock; Restasis; | |
| | | SangCya; | |
| | | Sigmasporin; | |
| | | Cipol-N; Gengraf; | |
| | | Sandimmun | |
| | | Neoral; | |
| | | Sandimmune; | |
| | | Cyclokat; | |
| | | Consupren; Neoral | |
| Launched- | 5-Hydroxy-1-(beta-D- | HE-69, Mizoribine, | EP 0457336; JP 1993310578; |
| 1984 | ribofuranosyl)imidazole-4- | Bredinin | JP 2001278790; JP 2007106710; |
| | carboxamide | | WO 1993002683; WO 2001087324; |
| | | | WO 2004012746; WO 2004083186; |
| | | | WO 2005016235; WO 2008026729 |
| Launched- | 1,8-Diethyl-1,3,4,9- | RAK-591; AY- | EP 0266114; EP 0748628; |
| 1985 | tetrahydropyrano[3,4-b]indole-1- | 24236, Etodolac; | JP 2005213191; JP 2009120599; |
| | acetic acid | Etodolic acid, | US 2004147582; U.S. Pat. No. 3,843,681; |
| | | Lodine XL; Lodine | U.S. Pat. No. 4,585,877; U.S. Pat. No. 4,966,768; |
| | | SR; Lodine; | U.S. Pat. No. 6,545,034; U.S. Pat. No. 6,573,292; |
| | | Hypen; Ultradol; | U.S. Pat. No. 6,586,005; WO 1996038452; |
| | | Edolan; Osteluc | WO 2003059344; WO 2003080183; |
| | | | WO 2005072775; WO 2006081088; |
| | | | WO 2006081127; WO 2006102476; |
| | | | WO 2007004675; WO 2008136374; |
| | | | WO 2009075094; WO 2009116509 |
| Launched- | 4-(6-Methoxynaphthalen-2-yl)-2- | BRL-14777, | CA 1134384; EP 0003643; |
| 1985 | butanone | Nabumetone, | EP 0167062; EP 0376516; |
| | | Nabuser; Relifen; | EP 1064967; WO 1992004892; |
| | | Listran; Relafen; | WO 1992008451; WO 1992008452; |
| | | Arthaxan; Relifex | WO 1992018114; WO 1993014747; |
| | | | WO 1994009767; WO 1995002399; |
| | | | WO 1996040608; WO 2001013889; |
| | | | WO 2001058441 |
| Launched- | 11beta,21-Dihydroxy-2′-methyl-5′- | MDL-458; DL- | WO 2009019504; WO 2009019505; |
| 1985 | betaH-pregna-1,4-dieno[17,16- | 458IT; L-5458, | WO 2009040814 |
| | d]oxazole-3,20-dione 21-acetate | Oxazacort; | |
| | | Azacort; | |
| | | Deflazacort, | |
| | | Dezacor; Calcort; | |
| | | Deflan; Flantadin | |
| Launched- | 4-Chloro-2,2′-iminidibenzoic acid | CCA, Lobenzarit | DE 2526092; JP 1992128222 |
| 1986 | disodium salt | sodium, Carfenil | |
| Launched- | 2-[2-(2,6- | PRO-513; PRO- | EP 1721602; WO 2005027879; |
| 1986 | Dichlorophenylamino)phenyl]acetic | 571, Diclofenac | WO 2006133954; WO 2007121848; |
| | acid potassium salt | potassium, | WO 2007127207; WO 2007130507; |
| | | Catafast; Voltfast; | WO 2008141888; WO 2009118764 |
| | | Zipsor; Cataflam; | |
| | | Cambia | |
| Launched- | 2(S)-Methylbutyric acid | L-154803; MK- | EP 0306210; EP 1123717; |
| 1987 | (1S,3R,7S,8S,8aR)-8-[2-[4(R)- | 803, Monakolin K; | EP 1127573; EP 1555021; |
| | hydroxy-6-oxotetrahydropyran-2(R)- | Mevinolin; | EP 1738757; U.S. Pat. No. 4,231,938; |
| | yl]ethyl]-3,7-dimethyl-1,2,3,7,8,8a- | Lovastatin, | U.S. Pat. No. 5,916,595; U.S. Pat. No. 6,080,778; |
| | hexahydronaphthalen-1-yl ester; 2- | Lovalord; | U.S. Pat. No. 6,379,696; |
| | Methylbutyric acid [1S- | Nergadan; Altocor; | |
| | [1alpha(R*),3alpha,7beta,8beta(2S*,4S*), | Rextat; Altoprev; | |
| | 8abeta]]-1,2,3,7,8,8a-hexahydro- | Liposcler; Artein; | |
| | 3,7-dimethyl-8-[2-(tetrahydro-4- | Mevinacor; | |
| | hydroxy-6-oxo-2H-pyran-2-yl)ethyl]- | Mevacor | |
| | 1-naphthalenyl ester; (S)-2- | | |
| | Methylbutyric acid,8-ester with | | |
| | (4R,6R)-6-[2-[(1S,2S,6R,8S,8aR)- | | |
| | 1,2,6,7,8,8a-hexahydro-8-hydroxy- | | |
| | 2,6-dimethyl-1- | | |
| | naphthyl]ethyl]tetrahydro-4-hydroxy- | | |
| | 2H-pyran-2-one | | |
| Launched- | N-(2-Mercapto-2-methylpropionyl)-L- | SA-96; DE-019, | EP 0551180; JP 1992154721; |
| 1987 | cysteine | Bucillamine; | JP 1992154722; JP 1992342524; |
| | | Tiobutarit, Rimatil | JP 1999071272; U.S. Pat. No. 4,305,958; |
| | | | WO 2000078305 |
| Launched- | 4-Hydroxy-2-methyl-N-(2-pyridyl)- | Ro-120068, | DE 2537070; WO 2006116626 |
| 1987 | 2H-thieno[2,3-e]-1,2-thiazine-3- | Tenoxicam, | |
| | carboxamide 1,1-dioxide | Tilcotil; Liman; | |
| | | Mobiflex | |
| Launched- | An apheresis-based treatment | Prosorba | |
| 1987 | containing Staphylococcal Protein A | | |
| Launched- | 17,21-Bis(acetyloxy)-2-bromo- | Halopredone | GB 1499822 |
| 1988 | 6beta,9-difluoro-11beta- | acetate, Haloart | |
| | hydroxypregna-1,4-diene-3,20-dione; | | |
| | 2-Bromo-6beta,9-difluoro- | | |
| | 11beta,17,21-trihydroxypregna-1,4- | | |
| | diene-3,20-dione 17,21-diacetate | | |
| Launched- | S,S′-Didehydro-D-phenylalanyl- | NSC-685403; | EP 0029579; EP 0671413; |
| 1988 | cysteinyl-phenylalanyl-D-tryptophyl- | VAP-003; C2L; | EP 1040837; EP 1787658; |
| | lysyl-threonyl-cysteinyl-threoninol | SMS-995; SMS- | EP 2052716; GB 2234896; |
| | acetate salt; [R-(R*,R*)]-D- | 201995; VP-003, | US 2006204540; U.S. Pat. No. 5,688,530; |
| | Phenylalanyl-L-cysteinyl-L- | Octeotride SDI; | U.S. Pat. No. 5,753,618; U.S. Pat. No. 5,922,682; |
| | phenylalanyl-D-tryptophyl-L-lysyl-L- | Octreotide acetate; | U.S. Pat. No. 6,346,601; U.S. Pat. No. 6,521,599; |
| | threonyl-N-[2-hydroxy-1- | Octreotide LAR, | U.S. Pat. No. 6,852,688; |
| | (hydroxymethyl)propyl]-L- | Longastatina; | |
| | cysteinamide cyclic (2--7)-disulfide | Sandostatin; | |
| | acetate salt; D-Phenylalanyl-L- | Sandostatin LAR; | |
| | cysteinyl-L-phenylalanyl-D- | Longastatina LAR | |
| | tryptophyl-L-lysyl-L-threonyl-N- | | |
| | [(1R,2R)-2-hydroxy-1- | | |
| | (hydroxymethyl)propyl]-L- | | |
| | cysteinamide cyclic (2--7)-disulfide | | |
| | acetate salt | | |
| Launched- | Glycolic acid [2-(2,6- | YT-919, | CH 682747; ES 2020146; |
| 1992 | dichloroanilino)phenyl]acetate ester; | Aceclofenac, | ES 2046141; U.S. Pat. No. 4,548,952; |
| | 2-(2,6- | Airtal; Beofenac; | WO 1999055660; WO 1999062865; |
| | Dichlorophenylamino)phenylacetic | Preservex; Gerbin; | WO 2005032516; WO 2006054315; |
| | acid carboxymethyl ester; 2-[2-(2,6- | Falcon | WO 2007138557; WO 2007140555 |
| | Dichlorophenylamino)phenylacetoxy]acetic | | |
| | acid | | |
| Launched- | (±)-2-(10-Oxo-10,11- | ZC-102; CN-100, | DE 2941869; JP 2001089370; |
| 1993 | dihydrodibenzo[b,f]thiepin-2- | Zaltoprofen, | WO 2007086692; WO 2007086694 |
| | yl)propionic acid; (±)-alpha-Methyl- | Soleton; Peon | |
| | 10-oxo-10,11- | | |
| | dihydrodibenzo[b,f]thiepin-2-acetic | | |
| | acid | | |
| Launched- | (1S,2S,3R,4S,7R,9S,10S,12R,15S)- | MPI-5018 (with | CA 2163837; EP 0400971; |
| 1993 | 4,12-bis(acetyloxy)-1,9-dihydroxy-15- | Matrix's anhydrous | EP 0522936; EP 0584001; |
| | {[(2R,3S)-2-hydroxy-3-phenyl-3- | delivery system); | EP 0700910; EP 1287854; |
| | (phenylformamido)propanoyl]oxy}- | OAS-PAC-100; | EP 1348430; EP 1700596; |
| | 10,14,17,17-tetramethyl-11-oxo-6- | SDP-013; ANX- | EP 1739184; EP 1946747; |
| | oxatetracyclo[11.3.1.0(3,10).0(4,7)]heptadec- | 513; DTS-301; | EP 1952789; EP 1961735; |
| | 13-en-2-yl benzoate; [2aR- | Nova-12005; | EP 1987813; EP 1990423; |
| | [2aalpha,4beta,4abeta,6beta,9alpha(2R, | BMS-181339; | EP 2025333; EP 2065054; |
| | 3S),11beta,12alpha,12aalpha,12balpha]]- | DHP-107; DHP- | US 2004072760; US 2004118007; |
| | 6,12b-Diacetoxy-9-(3-benzamido- | 208; EmPAC; | US 2004245662; US 2006073175; |
| | 2-hydroxy-3-phenylpropionyloxy)-12- | NSC-125973, | US 2006257326; US 2007196361; |
| | (benzoyloxy)-4,11-dihydroxy- | Paclitaxel; Intaxel, | US 2008175881; US 2008182776; |
| | 4a,8,13,13-tetramethyl- | Paclical; Paxceed; | US 2008193490; US 2008194500; |
| | 2a,3,4,4a,5,6,9,10,11,12,12a,12b- | OncoGel; Genexol- | US 2008213189; US 2008245375; |
| | dodecahydro-1H-7,11- | PM; Genexol-PM; | US 2008247957; US 2008255035; |
| | methanocyclodeca[3,4]benzo[1,2- | Genaxol; Pacligel; | US 2008262078; US 2008286372; |
| | b]oxet-5-one; | Nanotaxel; | US 2009082277; US 2009088393; |
| | (1S,2S,3R,4S,7R,9S,10S,12R,15S)- | Paxoral; Anzatax; | U.S. Pat. No. 4,857,653; U.S. Pat. No. 5,015,744; |
| | 4,12-bis(acetyloxy)-1,9-dihydroxy-15- | Genexol; Xorane; | U.S. Pat. No. 5,136,060; U.S. Pat. No. 5,200,534; |
| | {[(2R,3S)-2-hydroxy-3-phenyl-3- | Genetaxyl; Taxol; | U.S. Pat. No. 5,415,869; U.S. Pat. No. 5,416,225; |
| | (phenylformamido)propanoyl]oxy}- | Yewtaxan; Paxene | U.S. Pat. No. 5,445,809; U.S. Pat. No. 5,580,898; |
| | 10,14,17,17-tetramethyl-11-oxo-6- | | U.S. Pat. No. 5,670,537; U.S. Pat. No. 5,675,025; |
| | oxatetracyclo[11.3.1.0(3,10).0(4,7)]heptadec- | | U.S. Pat. No. 5,760,251; U.S. Pat. No. 5,861,302; |
| | 13-en-2-yl benzoate; 3(S)- | | U.S. Pat. No. 6,150,398; U.S. Pat. No. 6,461,637; |
| | Benzamido-2(R)- | | U.S. Pat. No. 6,538,020; U.S. Pat. No. 6,541,509; |
| | hydroxybenzenepropionic acid | | U.S. Pat. No. 6,664,288; U.S. Pat. No. 6,667,337; |
| | (2aR,4S,4aS,6R,9S,11S,12S,12aR,12bS)- | | |
| | 6,12b-diacetoxy-12-(benzoyloxy)- | | |
| | 4,11-dihydroxy-4a,8,13,13- | | |
| | tetramethyl-5-oxo- | | |
| | 2a,3,4,4a,5,6,9,10,11,12,12a,12b- | | |
| | dodecahydro-1H-7,11- | | |
| | methanocyclodeca[3,4]benzo[1,2- | | |
| | b]oxet-9-yl ester | | |
| Launched- | [3S(1′R,3′R,4′R),4R,5S,8R,12S,14S,15R, | FK-506; L-679934; | EP 0184162; EP 0240773; |
| 1993 | 16S,18R,19R,26aS]-8-Allyl-15,19- | LCP-Tacro; | EP 0315978; EP 0378318; |
| | epoxy-5,19-dihydroxy-3-[2-(4- | Modigraf; FR- | EP 0406791; EP 0483842; |
| | hydroxy-3-methoxycyclohexyl)-1(E)- | 900506, | EP 0484936; EP 1810675; |
| | methylvinyl]-14,16-dimethoxy- | Tacrolimus; | EP 1974722; JP 1991240726; |
| | 4,10,12,18-tetramethyl- | Fujimycin, Prograf; | JP 2008037808; US 2002013340; |
| | 1,4,5,6,7,8,11,12,13,14,15,16,17,18,19, | Protopic; | US 2002018776; US 2006154953; |
| | 20,21,23,24,25,26,26a-docosahydro- | Advagraf, Protopy; | US 2008161248; US 2009130198; |
| | 3H-pyrido[2,1- | Graceptor | US 2009155929; U.S. Pat. No. 4,940,797; |
| | c][1,4]oxaazacyclotricosine-1,7,20,21- | | U.S. Pat. No. 5,087,703; U.S. Pat. No. 5,194,378; |
| | tetraone; | | U.S. Pat. No. 5,196,437; U.S. Pat. No. 5,260,301; |
| | [1R,9S,12S(1′R,3′R,4′R),13R,14S,17R, | | U.S. Pat. No. 5,348,966; U.S. Pat. No. 5,385,907; |
| | 21S,23S,24R,25S,27R]-17-Allyl- | | U.S. Pat. No. 6,333,334; U.S. Pat. No. 6,346,537; |
| | 1,14-dihydroxy-12-[2-(4-hydroxy-3- | | U.S. Pat. No. 6,576,259; U.S. Pat. No. 6,833,353; |
| | methoxycyclohexyl)-1(E)- | | U.S. Pat. No. 7,063,857; |
| | methylvinyl]-23,25-dimethoxy- | | |
| | 13,19,21,27-tetramethyl-11,28-dioxa- | | |
| | 4-azatricyclo[22.3.1.0(4,9)]octacos- | | |
| | 18(E)-ene-2,3,10,16-tetraone; [3S- | | |
| | [3R*[E(1S*,3S*,4S*)],4S*,5R*,8S*,9E, | | |
| | 12R*,14R*,15S*,16R*,18S*,19S*,26aR*]]- | | |
| | 5,6,8,11,12,13,14,15,16,17,18,19,24,25, | | |
| | 26,26a-Hexadecahydro-5,19- | | |
| | dihydroxy-3-[2-(4-hydroxy-3- | | |
| | methoxycyclohexyl)-1- | | |
| | methylethenyl]-14,16-dimethoxy- | | |
| | 4,10,12,18-tetramethyl-8-(2- | | |
| | propenyl)-15,19-epoxy-3H- | | |
| | pyrido[2,1- | | |
| | c][1,4]oxaazacyclotricosine- | | |
| | 1,7,20,21(4H,23H)-tetraone | | |
| Launched- | 2-(4-Acetamidophenyl)acetic acid; 4- | MS-932, Actarit, | DE 3317107; WO 2006046007; |
| 1994 | (Acetylamino)benzeneacetic acid | Mover; Orcl | WO 2007004675 |
| Launched- | 4-Hydroxy-2-methyl-N-(5- | UH-AC 62XX, | DE 2756113; EP 0945131; |
| 1996 | methylthiazol-2-yl)-2H-1,2- | Meloxicam, | EP 1726301; EP 1810674; |
| | benzothiazine-3-carboxamide 1,1- | Movalis; Mobicox; | EP 1923392; JP 2007182400; |
| | dioxide | Tenaron; Mobic; | JP 2007197357; JP 2007269652; |
| | | Mobec; Parocin; | JP 2007297361; JP 2009114094; |
| | | Movatec | JP 2009203213; US 2004229038; |
| | | | US 2005026902; US 2007281927; |
| Launched- | 6-Chloro-4-hydroxy-2-methyl-N-(2- | CLTX; Ro-13- | DE 2838851; JP 1989316314; |
| 1997 | pyridyl)-2H-thieno[2,3-e]-1,2- | 9297; HN-10000; | JP 1989316315; JP 1990076816; |
| | thiazine-3-carboxamide 1,1-dioxide | TS-110, | WO 1998009654; WO 2006000228; |
| | | Lornoxicam; | WO 2006081088; WO 2006081127 |
| | | Chlortenoxicam, | |
| | | Safem; Acabel; | |
| | | Xefo; Lorcam; | |
| | | Telos; Taigalor; | |
| | | Xefocam | |
| Launched- | Pentahydrogen (OC-6-21)- | SHR-3644; | EP 0164843; WO 2008151226 |
| 1997 | [[[ethylenebis(nitrilodimethylene)]tetraphosphonato] | Sm-153-EDTMP; | |
| | (8-)- | CYT-424, | |
| | N,N′,O(P),O(P′),O(P″),O(P′″)]samarate(5-)- | Lexidronam Sm | |
| | 153Sm | 153; Samarium Sm | |
| | | 153 lexidronam, | |
| | | Quadramet | |
| Launched- | 2-[2-[1-Methyl-5-(4- | ST-679; MED-15, | BE 0896018; EP 0755679 |
| 1997 | methylbenzoyl)pyrrol-2- | Amtolmetin guacil, | |
| | yl]acetamido]acetic acid 2- | Eufans | |
| | methoxyphenyl ester; N-[(1-Methyl-5- | | |
| | p-toluoylpyrrol-2-yl)acetyl]glycine 2- | | |
| | methoxyphenyl ester; 2-[2-[1-Methyl- | | |
| | 2-(4-methylbenzoyl)pyrrol-5- | | |
| | yl]acetamido]acetic acid 2- | | |
| | methoxyphenyl ester | | |
| Launched- | Chimeric anti-CD20 antibody | IDEC-C2B8; R- | CN 1824307; US 2008213280; |
| 1997 | consisting of human IgG1-kappa | 105; IDEC-102; | US 2008233128; US 2009060913; |
| | constant regions and variable regions | RTX; RG-105, | U.S. Pat. No. 6,455,043; |
| | from murine monoclonal antibody | Rituximab, | |
| | IDEC-2B8; Immunoglobulin G | MabThera; Rituxan | |
| | (human-mouse monoclonal IDEC- | | |
| | C2B8 gamma1-chain anti-human | | |
| | antigen CD20), disulfide with human- | | |
| | mouse monoclonal IDEC-C2B8 | | |
| | kappa-chain, dimer | | |
| Launched- | 5-Methyl-N-[4- | RS-34821; SU- | CA 2140106; DE 2854439; |
| 1998 | (trifuoromethyl)phenyl]isoxazole-4- | 101; HWA-486, | DE 3534440; DE 4127737; |
| | carboxamide | Leflunomide, | EP 0413329; EP 0607774; |
| | | Arava | EP 0607775; EP 0607776; |
| | | | EP 0607777; EP 0617959; |
| | | | EP 0903345; EP 1275638; |
| | | | U.S. Pat. No. 5,610,173; U.S. Pat. No. 5,700,822; |
| | | | U.S. Pat. No. 6,331,555; |
| Launched- | Immunoglobulin G (human-mouse | TA-650, | JP 2008048629; JP 2009102390; |
| 1998 | monoclonal cA2 heavy chain anti- | Infliximab, | JP 2009225713; US 2005249735; |
| | human tumor necrosis factor), | Remicade; | US 2008124383; US 2009098136; |
| | disulfide with human-mouse | Avakine; CenTNF; | |
| | monoclonal cA2 light chain, dimer | cA2 | |
| Launched- | Recombinant fusion protein | TNFR:Fc; | JP 2006213659; US 2004235047; |
| 1998 | comprising the soluble human p75 | rhTNFR:Fc; | US 2008124383; US 2009098136; |
| | tumor necrosis factor (TNF) receptor | TNR-001, | U.S. Pat. No. 6,531,128; U.S. Pat. No. 6,800,300; |
| | linked to the Fc portion of human | Etanercept, Enbrel | |
| | IgG1; 1-235-Tumor necrosis factor | | |
| | receptor (human) fusion protein with | | |
| | 236-467-immunoglobulin G1 (human | | |
| | gamma1-chain Fc fragment) dimer | | |
| Launched- | Recombinant human interferon beta- | IFN-beta1a; | EP 1870107; WO 2009003905 |
| 1998 | 1a produced in Chinese hamster ovary | IFN-B-1a, | |
| | cells (CHO-K1) | Interferon beta-1a, | |
| | | Rebif | |
| Launched- | Acetyl-D-2-naphthylalanyl-D-4- | D-20761; SB-075; | EP 0299402; EP 1967202; |
| 1999 | chlorophenylalanyl-D-3- | NS-75B (pamoate | EP 2060580; US 2003044463; |
| | pyridylalanyl-seryl-tyrosyl-D- | salt); NS-75A | US 2004138138; WO 1995000168; |
| | citrullyl-leucyl-arginyl-prolyl-D- | (acetate); D-20453 | WO 1998018482; WO 1999055357; |
| | alaninamide acetate; Acetyl-D-2- | (trifluoroacetate); | WO 2000004897; WO 2002102401; |
| | naphthylalanyl-D-4- | SB-75, Cetrorelix | WO 2003011314; WO 2004056388 |
| | chlorophenylalanyl-D-3- | acetate, Cetrotide | |
| | pyridylalanyl-seryl-tyrosyl-D-N5- | | |
| | carbamoylornithyl-leucyl-arginyl- | | |
| | prolyl-D-alaninamide acetate | | |
| Launched- | (±)-5-[4-[2-(5-Ethylpyridin-2- | AA-10090; AD- | CA 2179584; EP 0193256; |
| 1999 | yl)ethoxy]benzyl]thiazolidine-2,4- | 4833 (free base); | EP 0506273; EP 0930076; |
| | dione monohydrochloride | U-72107E (as | EP 1903042; JP 2001294537; |
| | | AcOH solvate); | JP 2001316292; JP 2007197427; |
| | | U-72107A; | JP 2008001690; JP 2009013091; |
| | | U-72107 (free | JP 2009107944; JP 2009153514; |
| | | base), Pioglitazone | JP 2009190991; JP 2009196936; |
| | | hydrochloride (free | JP 2009203197; US 2008182880; |
| | | base), Glustin; | U.S. Pat. No. 5,356,913; U.S. Pat. No. 6,552,055; |
| | | Actos; Zactos | U.S. Pat. No. 6,673,823; U.S. Pat. No. 6,677,363; |
| Launched- | (±)-5-[4-[2-[N-Methyl-N-(2- | BRL-49653C; | EP 0306228; EP 0930076; |
| 1999 | pyridyl)amino]ethoxy]benzyl]thiazolidine- | SB-210232 | EP 1903042; EP 1905761; |
| | 2,4-dione maleate | ((+)-enantiomer, | JP 2001199887; JP 2008001690; |
| | | free base); | JP 2008195625; JP 2009209106; |
| | | SB-206846 | US 2005080114; US 2008207711; |
| | | ((−)-enantiomer, | U.S. Pat. No. 6,515,132; U.S. Pat. No. 6,552,055; |
| | | free base), | U.S. Pat. No. 6,673,823; U.S. Pat. No. 6,756,360; |
| | | Rosiglitazone | |
| | | maleate ((−)- | |
| | | enantiomer, free | |
| | | base), Nyracta; | |
| | | Venvia; Avandia | |
| Launched- | 4-[5-(4-Methylphenyl)-3- | YM-177; TPI-336 | EP 11064948; EP 1525883; |
| 1999 | (trifluoromethyl)pyrazol-1- | (crystalline); AI- | EP 1726301; EP 1743654; |
| | yl]benzenesulfonamide | 525; SC-58635; | EP 2085080; EP 2105130; |
| | | CEP-33222, | EP 2111854; JP 2005053888; |
| | | Celecoxib, | JP 2008201702; US 2002192201; |
| | | Celecox; Celebra; | US 2003157061; US 2005026902; |
| | | Solexa; Onsenal; | US 2006167075; US 2006178347; |
| | | Celebrex | US 2008234491; U.S. Pat. No. 5,563,165; |
| | | | U.S. Pat. No. 5,760,068; U.S. Pat. No. 5,972,986; |
| | | | U.S. Pat. No. 6,403,630; U.S. Pat. No. 6,440,963; |
| | | | U.S. Pat. No. 6,573,290; U.S. Pat. No. 6,777,424; |
| Launched- | 1-Hydroxy-2-(1-imidazolyl)ethylene- | CGP-42446 | AU 8776256; AU 8781453; |
| 2000 | 1,1-diphosphonic acid monohydrate | (anhydrous); CGP- | EP 0407344; EP 0550385; |
| | | 42446A (disodium | EP 1127573; EP 1925621; |
| | | salt); MER-101; | EP 1972341; US 2006128960; |
| | | CGP-42446B | US 2006258625; US 2007066569; |
| | | (triNa salt, | US 2008146489; WO 1995030421; |
| | | hydrate); ZOL-446, | WO 2001097788; WO 2002043738; |
| | | Zoledronic acid | WO 2002087555; WO 2003035081; |
| | | monohydrate; | WO 2003072097; WO 2003093282; |
| | | Zoledronate, | WO 2003097655; WO 2004012728; |
| | | Aclasta; Reclast; | WO 2004024165; WO 2004024166; |
| | | Orazol; Zometa | WO 2004035060; WO 2004075860; |
| | | | WO 2005005447; WO 2005014006; |
| | | | WO 2005025551; WO 2005053709; |
| | | | WO 2005063218; WO 2006006720; |
| | | | WO 2006134603; WO 2007015122; |
| | | | WO 2007016982; WO 2007028020; |
| | | | WO 2007032808; WO 2007069049; |
| | | | WO 2007081879; WO 2007083240; |
| | | | WO 2007124274; WO 2007125521; |
| | | | WO 2008003864; WO 2008023184; |
| | | | WO 2008040763; WO 2008060734; |
| | | | WO 2008116133; WO 2009112493; |
| | | | WO 2009112653; WO 2009115652; |
| | | | WO 2009128918 |
| Launched- | Recombinant human interleukin-1 | IL-1ra; rhIL-1ra, | WO 1995016706; WO 1997028828; |
| 2001 | receptor antagonist; A recombinant | Anakinra, Kineret; | WO 1998024477; WO 2001056606; |
| | nonglycosylated human interleukin-1 | Antril | WO 2001089549; WO 2002036152; |
| | receptor antagonist isolated from | | WO 2003022213; WO 2003045400; |
| | human monocytes and cloned and | | WO 2005023193; WO 2005063290; |
| | expressed in Escherichia coli; N2-L- | | WO 2006014444; WO 2006028939; |
| | Methionylinterleukin 1 receptor | | WO 2006059108; WO 2008021237; |
| | antagonist (human isoform x reduced) | | WO 2008112772; WO 2009025763; |
| | | | WO 2009053098; WO 2009108890 |
| Launched- | Holmium-166 [166Ho]-chitosan | DW-166HC, | WO 2005023316 |
| 2001 | complex | Milican | |
| Launched- | 5-Chloro-3-[4- | L-791456; | U.S. Pat. No. 6,040,319; U.S. Pat. No. 6,441,002; |
| 2002 | (methylsulfonyl)phenyl]-2-(6- | MK-0663; | WO 1998003484; WO 1999020110; |
| | methylpyridin-3-yl)pyridine; 5- | MK-663, | WO 1999045913; WO 1999059635; |
| | Chloro-6′-methyl-3-[4- | Etoricoxib, | WO 2000012093; WO 2000025779; |
| | (methylsulfonyl)phenyl]-2,3′- | Arcoxia; Tauxib; | WO 2001015687; WO 2001060365; |
| | bipyridine | Nucoxia | WO 2001087343; WO 2001091856; |
| | | | WO 2001092230; WO 2002005815; |
| | | | WO 2002022124; WO 2002087584; |
| | | | WO 2002089798; WO 2003039542; |
| | | | WO 2003049720; WO 2003088959; |
| | | | WO 2003094924; WO 2004039371; |
| | | | WO 2004045509; WO 2005000238; |
| | | | WO 2005000294; WO 2005000297; |
| | | | WO 2005007106; WO 2005007156; |
| | | | WO 2005018569; WO 2005037193; |
| | | | WO 2005039565; WO 2005051378; |
| | | | WO 2005085199; WO 2006137839 |
| Launched- | D-gamma-Glutamyl-D-tryptophan | D-iEW, | U.S. Pat. No. 5,736,519; U.S. Pat. No. 6,103,699; |
| 2002 | | Timodepressin, | WO 1996040740; WO 2008064465; |
| | | Thymodepressin | WO 2008098355 |
| Launched- | Immunoglobulin G1, anti-(human | anti-CD11a MAb; | WO 1998023761; WO 2000056363; |
| 2003 | CD11a [antigen]) (human-mouse | hu1124; | WO 2005113003; WO 2005123777; |
| | monoclonal hu1124, gamma1-chain), | Humanized | WO 2008016633; WO 2008124937 |
| | disulfide with human-mouse | MHM24, | |
| | monoclonal hu1124 light chain, dimer | Efalizumab, | |
| | | Xanelim; Raptiva | |
| Launched- | Immunoglobulin G1 (human | LU-200134; D2E7, | US 2004151722; US 2007041905; |
| 2003 | monoclonal D2E7 heavy chain anti- | Adalimumab, | US 2008124383; WO 1997029131; |
| | human tumor necrosis factor), | Trudexa; Humira | WO 2002100330; WO 2003045400; |
| | disulfide with human monoclonal | | WO 2004004633; WO 2004050683; |
| | D2E7 kappa-chain, dimer | | WO 2004071527; WO 2004098578; |
| | | | WO 2004105798; WO 2005000227; |
| | | | WO 2005038056; WO 2005110452; |
| | | | WO 2005115456; WO 2006041970; |
| | | | WO 2006122187; WO 2006125229; |
| | | | WO 2006138690; WO 2007024705; |
| | | | WO 2007120626; WO 2007120651; |
| | | | WO 2007120656; WO 2007120720; |
| | | | WO 2007120823; WO 2008021237; |
| | | | WO 2008056198; WO 2008124937; |
| | | | WO 2008142405; WO 2008147938; |
| | | | WO 2008150490; WO 2008150491; |
| | | | WO 2008154543; WO 2009003905; |
| | | | WO 2009050168; WO 2009073569; |
| | | | WO 2009073575; WO 2009090189; |
| | | | WO 2009117547; WO 2009132050 |
| Launched- | [1R,9S,12S[1′R(1″S,3″R,4″R)],15R,18R, | SDZ-RAD; RAD- | US 2008286372; US 2008300669; |
| 2004 | 19R,21R,23S,30S,32S,35R]-1,18- | 001; NVP-RAD- | US 2009155929; U.S. Pat. No. 7,438,722; |
| | Dihydroxy-12-[2-[4-(2- | 001; RAD-001C, | WO 1994009010; WO 1994024304; |
| | hydroxyethoxy)-3- | Everolimus, | WO 1995007468; WO 1997003654; |
| | methoxycyclohexyl]-1-methylethyl]- | Certican; Afinitor | WO 1997035575; WO 1998004279; |
| | 19,30-dimethoxy-15,17,21,23,29,35- | | WO 2000033878; WO 2001051049; |
| | hexamethyl-11,36-dioxa-4- | | WO 2002066019; WO 2003090818; |
| | azatricyclo[30.3.1.0(4,9)]hexatriaconta- | | WO 2004012768; WO 2005034916; |
| | 16(E),24(E),26(E),28(E)-tetraene- | | WO 2005049021; WO 2005053661; |
| | 2,3,10,14,20-pentaone; 40-O-(2- | | WO 2005110480; WO 2005117880; |
| | Hydroxyethyl)rapamycin; | | WO 2006014270; WO 2006053754; |
| | (3S,6R,7E,9R,10R,12R,14S,15E,17E, | | WO 2006060331; WO 2006065780; |
| | 19E,21S,23S,26R,27R,34aS)- | | WO 2006071966; WO 2006075165; |
| | 9,10,12,13,14,21,22,23,24,25,26,27,32, | | WO 2006102111; WO 2006122053; |
| | 33,34,34a-Hexadecahydro-9,27- | | WO 2006124739; WO 2006128660; |
| | dihydroxy-3-[2-[(1S,3R,4R)-4-(2- | | WO 2007010012; WO 2007011880; |
| | hydroxyethoxy)-3- | | WO 2007057457; WO 2007057466; |
| | methoxycyclohexyl]-1(R)- | | WO 2007059106; WO 2007068462; |
| | methylethyl]-10,21-dimethoxy- | | WO 2007080124; WO 2007088034; |
| | 6,8,12,14,20,26-hexamethyl-23,27- | | WO 2008016633; WO 2008022761; |
| | epoxy-3H-pyrido[2,1- | | WO 2008066783; WO 2008106223; |
| | c][1,4]oxaazacyclohentriacontine- | | WO 2009046436; WO 2009078875 |
| | 1,5,11,28,29(4H,6H,31H)-pentaone | | |
| Launched- | 2-[2-(2-Chloro-6-fluorophenylamino)- | COX-189, | WO 1999011605; WO 2001023346; |
| 2005 | 5-methylphenyl]acetic acid | Lumiracoxib, | WO 2002020090; WO 2003020261; |
| | | Prexige | WO 2003033001; WO 2003035047; |
| | | | WO 2003037341; WO 2003039599; |
| | | | WO 2003061645; WO 2003072097; |
| | | | WO 2003074041; WO 2003090737; |
| | | | WO 2004045509; WO 2004054575; |
| | | | WO 2004080451; WO 2004093856; |
| | | | WO 2004103357; WO 2005007106; |
| | | | WO 2005007156; WO 2005037193; |
| | | | WO 2005037266; WO 2005039565; |
| | | | WO 2005097096; WO 2006017354; |
| | | | WO 2006100213; WO 2008156645; |
| | | | WO 2009010529 |
| Launched- | Immunoglobulin G1, anti-(human | RG-1569; Anti- | U.S. Pat. No. 6,723,319; WO 1996011020; |
| 2005 | interleukin 6 receptor) (human-mouse | IL-6 receptor | WO 1996012503; WO 2002080969; |
| | monoclonal MRA heavy chain), | MAb; rhPM-1; R- | WO 2004096273; WO 2005028514; |
| | disulfide with human-mouse | 1569; Anti-IL-6R | WO 2005037315; WO 2005061000; |
| | monoclonal MRA kappa-chain, dimer; | MAb; MRA, | WO 2005080429; WO 2007061029; |
| | Humanized anti-human interleukin-6 | Tocilizumab; | WO 2008078715; WO 20081354 19; |
| | receptor (anti-hIL-6R) monoclonal | Atlizumab, | WO 2008156807; WO 2009052454; |
| | antibody derived from the murine PM- | RoActemra; | WO 2009084659 |
| | 1 antibody | Actemra | |
| Launched- | Extract of Cannabis sativa L. | OPC-33300; | US 2002111377; WO 2003037306; |
| 2005 | containing tetrahydrocannabinol | THC-CBD; | WO 2004016246 |
| | (THC) and cannabidiol (CBD) as its | GW-1000-02, | |
| | principal cannabinoid components; | Dronabinol/cannab | |
| | Highly characterized botanical extract | idiol; Nabiximols; | |
| | of a defined chemotype of Cannabis | Cannabis sativa L. | |
| | sativa L. The major chemical | extract, Sativex | |
| | constituents are the cannabinoids, | | |
| | delta-9-tetrahydrocannabinol (THC) | | |
| | and cannabidiol (CBD). Important | | |
| | minor constituents are related | | |
| | cannabinoids and non-cannabinoid | | |
| | components alpha- and trans- | | |
| | caryophyllenes | | |
| Launched- | Humanized monoclonal antibody | AN100226, | EP 2085407; US 2008311119; |
| 2006 | directed against alpha4 integrin; | Natalizumab, | WO 2003072040; WO 2004071439; |
| | Immunoglobulin G4 (human-mouse | Antegren; Tysabri; | WO 2005000244; WO 2005099776; |
| | monoclonal AN100226 gamma-chain | Antegran | WO 2005113003; WO 2006023649; |
| | anti-human integrin 4), disulfide with | | WO 2006036371; WO 2006052773; |
| | human-mouse monoclonal AN100226 | | WO 2006055871; WO 2006060787; |
| | light chain, dimer | | WO 2006067134; WO 2006089066; |
| | | | WO 2006107962; WO 2007103112; |
| | | | WO 2008036236; WO 2008157409; |
| | | | WO 2009025617; WO 2009103791 |
| Launched- | Fusion protein consisting of the | CTLA4-Ig; | CA 2146895; WO 2001095928; |
| 2006 | extracellular domain of human | BMS-188667, | WO 2002002638; WO 2002058729; |
| | cytotoxic T-lymphocyte-associated | Abatacept, Orencia | WO 2005016266; WO 2007134147; |
| | antigen 4 (CTLA-4) linked to the | | WO 2008016633 |
| | modified Fc (hinge, CH2 and CH3 | | |
| | domains) portion of human | | |
| | immunoglobulin G1 (IgG1); 1-25- | | |
| | Oncostatin M (human precursor) | | |
| | fusion protein with CTLA-4 (antigen) | | |
| | fusion protein with immunoglobulin | | |
| | G1 (human heavy chain fragment) | | |
| Launched- | (3S,6R,7E,9R,10R,12R,14S,15E,17E, | CCI-779; | US 2003153593; US 2006178392; |
| 2007 | 19E,21S,23S,26R,27R,34aS)- | NSC-683864, | US 2006199253; US 2007104721; |
| | 9,10,12,13,14,21,22,23,24,25,26,27,32, | Temserolimus; | U.S. Pat. No. 5,362,718; WO 2001023395; |
| | 33,34,34a-Hexadecahydro-9,27- | Temsirolimus, | WO 2002013802; WO 2002040000; |
| | dihydroxy-3-[2-[(1S,3R,4R)-4- | Torisel | WO 2002098416; WO 2003020266; |
| | hydroxy-3-methoxycyclohexyl]-1(R)- | | WO 2004011000; WO 2004026280; |
| | methylethyl]-10,21-dimethoxy- | | WO 2004071511; WO 2004078133; |
| | 6,8,12,14,20,26-hexamethyl-23,27- | | WO 2004093854; WO 2005011688; |
| | epoxy-3H-pyrido[2,1- | | WO 2005016935; WO 2005023254; |
| | c][1,4]oxaazacyclohentriacontine- | | WO 2005046681; WO 2005049021; |
| | 1,5,11,28,29(4H,6H,31H)-pentaone 4′- | | WO 2005070393; WO 2005087265; |
| | [2,2-bis(hydroxymethyl)propionate]; | | WO 2005105811; WO 2005105812; |
| | Rapamycin 42-[2,2- | | WO 2005117880; WO 2006050461; |
| | bis(hydroxymethyl)propionate]; | | WO 2006071966; WO 2006102111; |
| | [1R,9S,12S[1′R(1″R,3″R,4″R)],15R,18R, | | WO 2006119018; WO 2006122053; |
| | 19R,21R,23S,30S,32S,35R]-1,18- | | WO 2007010012; WO 2007059106; |
| | Dihydroxy-12-[2-[4-[3-hydroxy-2- | | WO 2008016633; WO 2008066783; |
| | (hydroxymethyl)-2- | | WO 2008124125; WO 2009046436; |
| | methylpropionyloxy]-3- | | WO 2009058895; WO 2009073115; |
| | methoxycyclohexyl]-1-methylethyl]- | | WO 2009111698; WO 2009117669; |
| | 19,30-dimethoxy-15,17,21,23,29,35- | | WO 2009140675 |
| | hexamethyl-11,36-dioxa-4- | | |
| | azatricyclo[30.3.1.0(4,9)]hexatriaconta- | | |
| | 16(E),24(E),26(E),28(E)-tetraene- | | |
| | 2,3,10,14,20-pentaone | | |
| Launched- | Immunoglobulin, anti-(human | h5G1.1; 5G1.1, | US 2005169921; US 2005271660; |
| 2007 | complement C5 alpha-chain) (human- | Eculizumab, Soliris | WO 1995029697; WO 2003081206; |
| | mouse monoclonal 5G1.1 heavy | | WO 2004022096; WO 2004024098; |
| | chain), disulfide with human-mouse | | WO 2005110481; WO 2006107708; |
| | monoclonal 5G1.1 light chain, dimer | | WO 2007002571; WO 2007106585; |
| | | | WO 2007130031; WO 2008030505; |
| | | | WO 2008048689; WO 2009014633; |
| | | | WO 2009105217 |
| Launched- | 4,4-Difluoro-N-[3-[(1R,3exo,5S)-3-(3- | MVC; UK-427857, | WO 2000038680; WO 2001090106; |
| 2007 | isopropyl-5-methyl-4H-1,2,4-triazol- | Maraviroc, | WO 2003100427; WO 2005016226; |
| | 4-yl)-8-azabicyclo[3.2.1]oct-8-yl]- | Selzentry; Celsentri | WO 2006055660; WO 2007085567; |
| | 1(S)-phenylpropyl]- | | WO 2007113648; WO 2007144720; |
| | cyclohexanecarboxamide | | WO 2008099278; WO 2008132128 |
| Launched- | Immunoglobulin, anti-(human tumor | PHA-738144; | US 2003157061; US 2008124383; |
| 2008 | necrosis factor alpha) Fab′ fragment | CDP-870, | WO 2001094585; WO 2003045400; |
| | (human-mouse monoclonal CDP870 | Certolizumab | WO 2004019860; WO 2004019861; |
| | heavy chain), disulfide bonded with | pegol, Cimziat; | WO 2004053064; WO 2004067006; |
| | human-mouse monoclonal CDP870 | Cimzia | WO 2004071527; WO 2004098578; |
| | light chain, pegylated at Cys-221 on | | WO 2005123772; WO 2009073575 |
| | the heavy chain | | |
| Launched- | Interleukin 1 receptor accessory | IL-1 Trap, IL-1 | EP 1229047; WO 2004039951; |
| 2008 | protein (human extracellular domain | Cytokine Trap; | WO 2004098596; WO 2004098605; |
| | fragment) fusion protein with type I | Rilonacept; | WO 2004100987; WO 2005117945; |
| | interleukin 1 receptor (human | Interleukin-1 Trap, | WO 2006023665; WO 2006076673; |
| | extracellular domain fragment) fusion | Arcalyst | WO 2006084145; WO 2007042524; |
| | protein with immunoglobulin G1 | | WO 2008051496 |
| | (human Fc fragment), homodimer; | | |
| | [653-Glycine][human interleukin-1 | | |
| | receptor accessory protein-(1-339)- | | |
| | peptide (extracellular domain | | |
| | fragment) fusion protein with human | | |
| | type 1 interleukin-1 receptor-(5-316)- | | |
| | peptide (extracellular domain | | |
| | fragment) fusion protein with human | | |
| | immunoglobulin G1-(229 C-terminal | | |
| | residues)-peptide (Fc fragment)], | | |
| | (659-659′:662-662′)-bisdisulfide | | |
| | dimer; Dimeric fusion protein | | |
| | consisting of the ligand-binding | | |
| | domains of the extracellular portions | | |
| | of the human interleukin-1 receptor | | |
| | component (IL-1RI) and IL-1 receptor | | |
| | accessory protein (IL-1RAcP) linked | | |
| | in-line to the Fc portion of human | | |
| | IgG1, expressed in recombinant | | |
| | Chinese hamster ovary (CHO) cells | | |
| | and with a molecular weight of | | |
| | approximately 251 kDa | | |
| Launched- | 1,1′-[1,4- | JM-2987 (as | EP 0389359; US 2002039993; |
| 2009 | Phenylenebis(methylene)]bis-1,4,8,11- | 8HBr•2H2O); | U.S. Pat. No. 6,365,583; WO 1993012096; |
| | tetraazacyclotetradecane | SDZ-SID-791; | WO 1996030349; WO 1996034860; |
| | octahydrochloride dihydrate; 1,4- | SID-791; JLK-169 | WO 1999065507; WO 2000002870; |
| | Bis(1,4,8,11-tetraazacyclotetradec-1- | (anhydrous, free | WO 2000006086; WO 2000028987; |
| | ylmethyl)benzene octahydrochloride | base); JM-3100 | WO 2002026721; WO 2003011277; |
| | dihydrate | (former code | WO 2004093817; WO 2005000333; |
| | | name); AMD- | WO 2005002522; WO 2006020891; |
| | | 3100, Plerixafor | WO 2006116185; WO 2006137934; |
| | | hydrochloride, | WO 2007022523; WO 2007047882; |
| | | Mozobil | WO 2007106063; WO 2008017025; |
| | | | WO 2008019371; WO 2009108360 |
| Launched- | Immunoglobulin G1, anti-[Homo | ACZ-885, | WO 2002016436; WO 2006119942; |
| 2009 | sapiens interleukin 1beta (IL-1B)] | Canakinumab, | WO 2007050607; WO 2007120828; |
| | human monoclonal ACZ885; gamma1 | Ilaris | WO 2008145664 |
| | heavy chain (Homo sapiens VH- | | |
| | IGHG1*03) (221-214′)-disulfide with | | |
| | kappa light chain (Homo sapiens V- | | |
| | KAPPA-IGKC*01); (227-227″:230-230″)- | | |
| | bisdisulfide dimer; | | |
| | Immunoglobulin G1, anti-(human | | |
| | interleukin 1beta) (human clone | | |
| | ACZ885 heavy chain V region); | | |
| | Immunoglobulin G1, anti-(human | | |
| | interleukin-1 beta (IL-1beta)) human | | |
| | monoclonal ACZ885; (1Glu > Glp)- | | |
| | gamma1 heavy chain (221-214′)- | | |
| | disulfide with kappa light chain, dimer | | |
| | (227-227″:230-230″)-bisdisulfide | | |
| Launched- | Immunoglobulin G1, anti-(human | HuMax-CD20; | US 2006233797; US 2009169550; |
| 2009 | CD20 (antigen))(human monoclonal | GSK-1841157 | WO 2004035607; WO 2005103081; |
| | HuMax-CD20 heavy chain), disulfide | (infusion); 2F2, | WO 2006076651; WO 2007117600; |
| | with human monoclonal HuMax- | Ofatumumab, | WO 2008003319; WO 2008118736; |
| | CD20 kappa-chain, dimer | Arzerra | WO 2009009407; WO 2009052830 |
| Launched- | Immunoglobulin G1, anti-(human | CNTO-148, | WO 2006125229; WO 2007056540; |
| 2009 | tumor necrosis factor alpha) (human | Golimumab, | WO 2008147938; WO 2008150491; |
| | monoclonal CNTO 148 gamma1- | Simponi | WO 2008154543; WO 2009073575; |
| | chain), disulfide with human | | WO 2009117547 |
| | monoclonal CNTO 148 kappa-chain, | | |
| | dimer | | |
| Phase 0 | | AB-224050; | |
| | | ABR-224050 | |
| Phase I | N-[4-(2,4-Diaminopteridin-6- | MX-68 | JP 2002370985; WO 1992003436; |
| | ylmethyl)-3,4-dihydro-2H-1,4- | | WO 1994014810; WO 1996030019; |
| | benzothiazin-7-ylcarbonyl]-L-(3′- | | WO 1997034606; WO 2000000492; |
| | homo)glutamic acid; 2(S)-[4-(2,4- | | WO 2001012198 |
| | Diaminopteridin-6-ylmethyl)-3,4- | | |
| | dihydro-2H-1,4-benzothiazin-7- | | |
| | ylcarboxamido]adipic acid | | |
| Phase I | 2-[8-[2-[6-(Methylamino)pyridyl-2- | SB-273005 | WO 1998014192 |
| | ylethoxy]-3-oxo-2-(2,2,2- | | |
| | trifluoroethyl)-2,3,4,5-tetrahydro-1H- | | |
| | 2-benzazepin-4-(S)-yl]acetic acid | | |
| Phase I | N-(2,6-Dichlorobenzoyl)-3-(2′,6′- | SB-683698; | WO 1999036393 |
| | dimethoxybiphenyl-4-yl)-L-alanine; | TR-14035 | |
| | N-(2,6-Dichlorobenzoyl)-4-(2,6- | | |
| | dimethoxyphenyl)-L-phenylalanine; | | |
| | 2(S)-(2,6-Dichlorobenzamido)-3- | | |
| | (2′,6′-dimethoxybiphenyl-4- | | |
| | yl)propionic acid | | |
| Phase I | | BB-2827 | |
| Phase I | 3-(3,5-Dimethoxyphenyl)-2-[4-[4- | CLX-0921 | US 2003181494; WO 1999058127; |
| | (2,4-dioxothiazolidin-5- | | WO 2001095859 |
| | ylmethyl)phenoxy]phenyl]-2- | | |
| | propenoic acid methyl ester | | |
| Phase I | Human anti-IL-15 monoclonal | HuMax-IL-15; | WO 2004076620; WO 2006047340 |
| | antibody | AMG-714; | |
| | | MAb146B7 | |
| Phase I | 6-Chloro-3-[3-(2,3- | SB-332235; | WO 2000035442; WO 2002079122 |
| | dichlorophenyl)ureido]-2- | 332235 | |
| | hydroxybenzenesulfonamide | | |
| Phase I | N-[3-[4-(2-Adamantyl)-3,5- | SSR-125329A; | WO 2000076953 |
| | dichlorophenyl]-2(Z)-propenyl]-N- | SR-125329; | |
| | cyclohexyl-N-ethylamine | SSR-125329 | |
| Phase I | | IPL-550260 | |
| Phase I | | SVT-2016 | |
| Phase I | Interleukin-1 (IL-1) receptor type II | IL-1RII, IL-1 | WO 2001087328 |
| | | receptor type II | |
| Phase I | Recombinant human interleukin-18 | rhIL-18bp, | U.S. Pat. No. 6,605,280; WO 2002032374; |
| | (IL-18) binding protein; Interleukin-18 | Tadekinig alfa | WO 2002092008; WO 2003080104; |
| | binding protein (human gene IL18BP | | WO 2005063290; WO 2006128908 |
| | isoform a precursor) | | |
| Phase I | 1-[5-Amino-1-(4-fluorophenyl)-1H- | RO-3201195; | WO 1999057101; WO 2001021591; |
| | pyrazol-4-yl]-1-[3-[2(S),3- | RO-320-1195 | WO 2005091891; WO 2006127678 |
| | dihydroxypropoxy]phenyl]methanone | | |
| Phase I | 2-(11-Oxo-6,11- | HP-720549; P-549; | |
| | dihydrodibenzo[b,e]oxepin-2-yl)acetic | HP-549, Isoxepac | |
| | acid | | |
| Phase I | Humanized monoclonal antibody to | TRX-1 | GB 2376467; WO 2006002377; |
| | the human T cell receptor CD4 | | WO 2006030200 |
| Phase I | | SB-281832 | |
| Phase I | 6-(2,4-Difluorophenoxy)-8-methyl-2- | R-1487 | WO 2002064594; WO 2005023201 |
| | (tetrahydropyran-4- | | |
| | ylamino)pyrido[2,3-d]pyrimidin- | | |
| | 7(8H)-one | | |
| Phase I | | R-594 | |
| Phase I | Dichloro[(4aS,13aS,17aS,21aS)-11,7- | M-40419; | EP 1420022; US 2002072512; |
| | nitrilo- | SC-72325A | WO 1998058636; WO 2003051458; |
| | 2,3,4,4a,5,6,7,12,13,13a,14,15,16,17,17a, | | WO 2006083508 |
| | 18,19,20,21,21a-eicosahydro-1H- | | |
| | dibenzo[b,h][1,4,7,10]tetraazacycloheptadecine- | | |
| | kappaN5,kappaN13,kappaN18,kappa | | |
| | N21,kappaN22]manganese | | |
| Phase I | N2-[(3R,4S,5S,6R)-5-Methoxy-4- | NSC-720735; | WO 2002042295; WO 2003092608; |
| | [2(R)-methyl-3(R)-(3-methyl-2- | PPI-2458, Metapro | WO 2007124005; WO 2008066641 |
| | butenyl)oxiran-2-yl]-1- | | |
| | oxaspiro[2.5]oct-6-yloxycarbonyl]-D- | | |
| | valinamide; 6-O-[N-[1(R)-Carbamoyl- | | |
| | 2-methylpropyl]carbamoyl]fumagillol | | |
| Phase I | | R-1295 | |
| Phase I | (+)-1(S)-[2,8- | RTI-1188; | EP 0553778; US 2004220221; |
| | Bis(trifluoromethyl)quinolin-4-yl]-1- | AD-452, | U.S. Pat. No. 6,664,397; WO 2002019994; |
| | [2(R)-piperidinyl]methanol | (+)-Mefloquine | WO 2004050625; WO 2005058872; |
| | hydrochloride | hydrochloride; | WO 2005089762 |
| | | (+)-erythro- | |
| | | Mefloquine | |
| | | hydrochloride | |
| Phase I | | RPI-MN | |
| Phase I | 3,4-Bis-O-(pyridin-3-ylcarbonyl)-1-O- | Niglizin | RU 1069403; RU 1804848; |
| | [(3beta,20beta)-29-hydroxy-11,29- | | RU 2073009 |
| | dioxoolean-12-en-3-yl]-2-O-[2,3,4- | | |
| | tris-O-(pyridin-3-ylcarbonyl)-beta-D- | | |
| | glucopyranuronosyl]-alpha-D- | | |
| | glucopyranosiduronic acid; | | |
| | (3beta,20beta)-29-Hydroxy-11,29- | | |
| | dioxoolean-12-en-3-yl 3,4-bis-O- | | |
| | (pyridin-3-ylcarbonyl)-2-O-[2,3,4-tris- | | |
| | O-(pyridin-3-ylcarbonyl)-beta-D- | | |
| | glucopyranuronosyl]-alpha-D- | | |
| | glucopyranosiduronic acid | | |
| Phase I | | PXS-25 | |
| Phase I | 3(S)-(2-Methoxyphenyl)-2(S)-methyl- | WAY-204688; | WO 2004099150 |
| | 3-(1-naphthyl)-2-[4-[3- | SIM-688 | |
| | (trifluoromethyl)phenyl]piperidin-1- | | |
| | ylcarbonyl]propanenitrile | | |
| Phase I | Highly purified Staphylococcal | SpA; PRTX-100, | |
| | Protein A (SpA) | Staphylococcal | |
| | | Protein A | |
| Phase I | | BT-061 | WO 2009112502; WO 2009121690; |
| | | | WO 2009124815 |
| Phase I | 2-O-(4-Carboxybutyl)-1-O-hexadecyl- | CI-201 | WO 2004106486 |
| | sn-glycero-3-phosphocholine | | |
| Phase I | | AVE-1701 | |
| Phase I | 2-(3-Phenyl-4,5-dihydroisoxazol-5- | GIT-027; | WO 2006097273; WO 2009132172 |
| | yl)acetic acid | VGX-1027 | |
| Phase I | | SAR-479746; | |
| | | MLN-0415 | |
| Phase I | Soluble homodimeric fusion | BR-3-FC; BR3-Fc, | WO 2007019618 |
| | glycoprotein consisting of sequences | Briobacept | |
| | from the extracellular domain of | | |
| | human BR3 (TNFRSF13C) fused to | | |
| | the Fc domain of a human IgG; | | |
| | Cytokine receptor BAFF-R (human | | |
| | extracellular domain-containing | | |
| | fragment BR3) fusion protein with | | |
| | immunoglobulin G1 (human Fc | | |
| | domain-containing fragment), dimer; | | |
| | Aspartyl[1-valine,20-asparagine,27- | | |
| | proline](human tumor necrosis factor | | |
| | receptor superfamily member 13C | | |
| | (BAFF receptor, BlyS receptor 3 or | | |
| | CD268 antigen)-(1-71)-peptidyl (part | | |
| | of the extracellular | | |
| | domain))valyl(human | | |
| | immunoglobulin G1 Fc fragment, | | |
| | Homo sapiens IGHG1-(104-329)- | | |
| | peptide), dimer (79-79′:82-82′)- | | |
| | bisdisulfide | | |
| Phase I | Human IgG1 monoclonal antibody | MT-203 | |
| | against human GM-CSF | | |
| Phase I | Fully human IgG4 anti-granulocyte- | CAM-3001 | WO 2007110631 |
| | macrophage colony stimulating factor | | |
| | receptor alpha chain (anti-GM- | | |
| | CSFRalpha) monoclonal antibody | | |
| Phase I | Fully HuCAL-derived human IgG1 | MOR-103 | |
| | monoclonal antibody directed against | | |
| | GM-CSF (granulocyte macrophage- | | |
| | colony stimulating factor) | | |
| Phase I | 4-[3-[5-Chloro-1-(diphenylmethyl)-2- | PLA-695, | US 2007021614; WO 2003048122; |
| | [2-[2- | Giripladib | WO 2006128142; WO 2008055136; |
| | (trifluoromethyl)benzylsulfonamido]ethyl]- | | WO 2008055141; WO 2008055146; |
| | 1H-indol-3-yl]propyl]benzoic | | WO 2008055148 |
| | acid | | |
| Phase I | | ARRY-438162; | |
| | | ARRY-162 | |
| Phase I | 3(S)-[N-[1(S)-(Isobutoxymethyl)-3- | VEL-0230; | WO 1999011640; WO 2004096785; |
| | methylbutyl]carbamoyl]oxirane-2(S)- | NC-2300 | WO 2007137149; WO 2009054454; |
| | carboxylic acid sodium salt | | WO 2009065098 |
| Phase I | | AMAP-102 | |
| Phase I | | TA-5493 | |
| Phase I | | R-348 | |
| Phase I | | GRC-4039 | |
| Phase I | | ELND-001 | |
| Phase I | Human IgG1 monoclonal antibody | MOR-04357 | WO 2006122797 |
| | against human GM-CSF (HuCAL- | | |
| | derived) | | |
| Phase I | Monoclonal antibody targeting MCSF | PD-0360324; | |
| | | PD-360324 | |
| Phase I | | QAL-964 | |
| Phase I | Human monoclonal antibody against | MDX-1342; 21D4 | WO 2009054863 |
| | CD19 | | |
| Phase I | Therapeutic vaccine consisting of | Rheumatoid | |
| | autologous synovial T-cells of | arthritis T-cell | |
| | rheumatoid arthritis patients and | vaccine | |
| | inactivated by irradiation | | |
| Phase I | Fusion protein comprising amino | hALK-Fc; | WO 2008057461 |
| | acids 22-120 of the human ALK1 | ACE-041 | |
| | protein, fused at the C-terminus to a | | |
| | linker and an IgG1 Fc region | | |
| Phase I | Humanized monoclonal antibody | Neutrazumab | |
| | against C5aR receptor | | |
| Phase I | | CCX-354 | |
| Phase I | 1-[3(R)-[4-Amino-3-(4- | PCI-32765 | US 2008076921; US 2008139582; |
| | phenoxyphenyl)-1H-pyrazolo[3,4- | | WO 2008039218; WO 2008054827; |
| | d]pyrimidin-1-yl]piperidin-1-yl]-2- | | WO 2008121742 |
| | propen-1-one | | |
| Phase I | Spiegelmer, L-enantiomeric RNA | 180-D1-036; | WO 2007093409 |
| | oligonucleotide, that binds to human | NOX-E36 | |
| | CCL2 (MCP-1) and whose sequence | | |
| | is: 5′- | | |
| | GCACGUCCCUCACCGGUGCAAG | | |
| | UGAAGCCGUGGCUCUGCG-3′ | | |
| Phase I | 6,7-Dimethoxy-2-[4-(4- | CPG-52364 | WO 2008152471 |
| | methylpiperazin-1-yl)phenyl]-N-[2-(4- | | |
| | morpholinyl)ethyl]quinazolin-4-amine | | |
| Phase I | | MP-435 | |
| Phase I | | REGN-88 | |
| Phase I | | AZD-8566 | |
| Phase I | | 1827771; | |
| | | GSK-1827771 | |
| Phase I | | SAR-153191 | |
| Phase I | | GT-418; | |
| | | GLPG-0259 | |
| Phase I | N-[4-[2-(2,4-Diaminoquinazolin-6- | CH-4051 | US 2005020833; US 2009253720; |
| | yl)ethyl]benzoyl]-4-methylene-L- | | U.S. Pat. No. 5,912,251; WO 2004045500; |
| | glutamic acid | | WO 2006029385 |
| Phase I | | BCT-197 | |
| Phase I | Anti-VAP-1 monoclonal antibodies | r8c10; BTT-1023 | WO 2008129124 |
| Phase I | | ILV-095 | |
| Phase I | | NN-8555; IPH- | |
| | | 2301; NNC-0142- | |
| | | 0000-0002 | |
| Phase I | | PF-4236921 | |
| Phase I | Humanized monoclonal antibody | BIIB-023 | |
| | targeting TWEAK | | |
| Phase I | | MDAM | |
| Phase I | Anti-IL-20 human monoclonal | Anti-IL-20 mAb | |
| | antibody | | |
| Phase I | | LAS-186323 | |
| Phase I | | PLX-3397 | |
| Phase I | | PF-4629991 | |
| Phase I | | EVT-401 | |
| Phase I | | PF-04236921 | |
| Phase I | | R-7424; RG-7424 | |
| Phase I | | R-7416; RG-7416 | |
| Phase I | | PRO-283698 | |
| Phase I/II | Two TNFbp molecules covalently | Pegylated | |
| | linked through a PEG linker | rHuTNFbp dimer; | |
| | | PEG-TNFbp, | |
| | | Pegylated onercept | |
| | | dimer | |
| Phase I/II | Allogeneic mononuclear cell therapy | LeukoVax | |
| Phase I/II | Tris[3-(hydroxy-kappaO)-2-methyl- | GaM, Gallium | US 2008175922; WO 1998004264; |
| | 4H-pyran-4-onato-kappaO4]gallium; | maltolate | WO 2005058331; WO 2007056440; |
| | Tris(3-hydroxy-2-methyl-4H-pyran-4- | | WO 2007087461 |
| | onato-O3,O4)gallium | | |
| Phase I/II | A PEGylated antibody fragment | CDP-484 | |
| | targeting the pro-inflammatory | | |
| | cytokine interleukin-1beta | | |
| Phase I/II | Humanized anti-TNFalpha | AME-527 | |
| | monoclonal antibody | | |
| Phase I/II | Anti-Fas IgM monoclonal antibody | DE-098; ARG-098 | |
| | that specifically targets the Fas (also | | |
| | known as APO-1 and CD95) molecule | | |
| Phase I/II | | AVE-9940 | |
| Phase I/II | Recombinant adeno-associated virus | rAAV- | WO 2007149115 |
| | (AAV) vector containing the cDNA | humanTNFR:Fc; | |
| | for the human tumor necrosis factor | tgAAC-94 | |
| | receptor-immunoglobulin (IgG1) Fc | | |
| | fusion (TNFR:Fc) gene (tgAAC94) | | |
| Phase I/II | Heterocomplex vaccine consisting of | hTNF-alpha-KLH, | WO 2000064937; WO 2004024189; |
| | human TNF-alpha complexed to KLH | TNF-alpha kinoid | WO 2005028513; WO 2007022813 |
| | (keyhole limpet hemocyanin) | | |
| Phase I/II | Human engineered IgG1k antibody | KB-002 | US 2009181020; WO 2008063898; |
| | against granulocyte macrophage | | WO 2008064321 |
| | colony-stimulating factor (GM-CSF) | | |
| Phase I/II | | CYT013-IL1bQb | |
| Phase I/II | | ATN-103 | |
| Phase I/II | | CDP-6038 | |
| Phase II | 2-[6(R)-Isopropenyl-3-methyl-2- | CBD, Cannabidiol, | U.S. Pat. No. 2,304,669; U.S. Pat. No. 6,630,507; |
| | cyclohexen-1(R)-yl]-5-pentyl-1,3- | Nabidiolex | WO 1999052524; WO 2004026802; |
| | benzenediol | | WO 2004041269; WO 2005077348; |
| | | | WO 2005102296; WO 2005120478; |
| | | | WO 2006017892; WO 2006037981; |
| | | | WO 2007042811; WO 2007083098; |
| | | | WO 2007148094; WO 2008024408; |
| | | | WO 2008024490; WO 2008050344; |
| | | | WO 2008120207; WO 2008129258; |
| | | | WO 2009007697; WO 2009013506; |
| | | | WO 2009043395; WO 2009087351; |
| | | | WO 2009093018 |
| Phase II | Interleukin 4 (human) | Sch-39400; hIL-4, | EP 0490006; U.S. Pat. No. 5,382,427; |
| | | Binetrakin; | WO 1987002990; WO 1991014450; |
| | | Recombinant | WO 1992011030; WO 1992011861; |
| | | human IL-4; | WO 1994004179; WO 1994004180; |
| | | Interleukin-4, | WO 1997013525 |
| | | human | |
| Phase II | 2-(1-Benzylindazol-3-ylmethoxy)-2- | AF-2838, Bindarit | U.S. Pat. No. 5,278,183; U.S. Pat. No. 6,534,534; |
| | methylpropanoic acid | | WO 1997016185; WO 2008061671 |
| Phase II | 2-[3-(Diethylamino)propyl]-8,8- | SK&F-106615-I2 | EP 0310321; WO 1993014760; |
| | dipropyl-2-azaspiro[4.5]decane | (dimaleate); | WO 1995003041; WO 1995003042; |
| | dihydrochloride; N,N-Diethyl-8,8- | SK&F-106615-A2, | WO 1995003049; WO 2004080408 |
| | dipropyl-2-azaspiro[4.5]decane-2- | Atiprimod | |
| | propanamine dihydrochloride | hydrochloride | |
| Phase II | (E)-5-(3,5-Di-tert-butyl-4- | S-2474 | EP 0595546; EP 0626377; |
| | hydroxybenzylidene)-2- | | JP 1995285926; JP 1996027134; |
| | ethylisothiazolidine 1,1-dioxide | | JP 1996217764; WO 1999021554; |
| | | | WO 1999021844 |
| Phase II | 4-(3,4-Dimethoxyphenyl)-6,7- | TAK-603 | EP 0608870; EP 0634169; |
| | dimethoxy-2-(1,2,4-triazol-1- | | WO 1997009984 |
| | ylmethyl)quinoline-3-carboxylic acid | | |
| | ethyl ester | | |
| Phase II | Hydrogen (SP-5-25)-[N,N- | 99mTc-RP-128 | WO 1996038185 |
| | dimethylglycyl-kappaN-L-seryl- | | |
| | kappaN-L-cysteinyl-kappaN,kappaS- | | |
| | glycyl-L-threonyl-L-lysyl-L-prolyl-L- | | |
| | prolyl-L-argininato(4-)]oxotechnetate(1-)- | | |
| | 99Tc | | |
| Phase II | N6-(3-Iodobenzyl)adenosine-5′-(N- | CF-101; IBMECA; | WO 2004030621; WO 2004045627; |
| | methyluronamide); 1-Deoxy-1-[N6- | IBMECA; | WO 2006011130; WO 2006048884; |
| | (3-iodobenzyl)adenin-9-yl]-beta-D- | RPR-113090; | WO 2007063538; WO 2007086044; |
| | ribofuranuronic acid methylamide | SI-615 | WO 2008023362; WO 2008075201; |
| | | | WO 2008111082 |
| Phase II | Complex of denatured protein | OHR-118; | US 2003206962; US 2004033244; |
| | molecules containing lipoproteins, | AVR-118, | U.S. Pat. No. 5,902,786; WO 1998046077; |
| | amino acids, polypeptides and | Substance R; | WO 1998046240; WO 1998046241; |
| | ribonucleic acids | Product R, | WO 1998046624; WO 2001000306; |
| | | Reticulose | WO 2001032933; WO 2001033188; |
| | | | WO 2002020043; WO 2002056833; |
| | | | WO 2006091222; WO 2006132623 |
| Phase II | Immunoglobulin G1, anti-[human | hLM609; MEDI- | US 2008181851; U.S. Pat. No. 6,590,079; |
| | alphaVbeta3 (CD51/CD61, | 522, Etaracizumab, | U.S. Pat. No. 6,596,850; WO 2002070007; |
| | CD51/GPIIIa, CD51/platelet | Abegrin; Vitaxin | WO 2003075741; WO 2004066957; |
| | membrane glycoprotein IIIa, | | WO 2006023420; WO 2006099481 |
| | vitronectin receptor)] humanized | | |
| | monoclonal antibody MEDI-522 | | |
| | (hLM609); gamma 1 heavy chain | | |
| | [humanized VH (Homo sapiens | | |
| | FR/Mus musculus CDR from clone | | |
| | LM609)-Homo sapiens IGHG1*03] | | |
| | (220-214′)-disulfide with kappa light | | |
| | chain [humanized V-KAPPA (Homo | | |
| | sapiens FR/Mus musculus CDR from | | |
| | clone LM609)-Homo sapiens | | |
| | IGKC*01]; (226-226″:229-229″)- | | |
| | bisdisulfide dimer; Immunoglobulin | | |
| | G1 (synthetic mouse NSO cell heavy | | |
| | chain variable region fragment), | | |
| | complex with immunoglobulin G1 | | |
| | (synthetic mouse NSO cell light chain | | |
| | variable region fragment); | | |
| | Immunoglobulin G1, anti-[Homo | | |
| | sapiens alphaVbeta3 integrin | | |
| | (CD51/CD61, CD51/GPIIIa, | | |
| | CD51/platelet membrane glycoprotein | | |
| | IIIa, vitronectin receptor)], humanized | | |
| | monoclonal antibody, MEDI-522 (or | | |
| | hLM609); gamma1 heavy chain (1-447) | | |
| | [humanized VH (Homo sapiens | | |
| | FR/Mus musculus CDR from clone | | |
| | LM609-Homo sapiens IGHJ5*01, | | |
| | L123 > T) [8.8.10] (1-117)-Homo | | |
| | sapiens IGHG1*03 (118-447)], (220-214′)- | | |
| | disulfide with kappa light chain | | |
| | (1′-214′) [humanized V-KAPPA | | |
| | (Homo sapiens FR/Mus musculus | | |
| | CDR from clone LM609-Homo | | |
| | sapiens IGKJ4*01) [6.3.9] (1′-107′)- | | |
| | Homo sapiens IGKC*01 (108′-214′)]; | | |
| | (226-226″:230-230″)-bisdisulfide | | |
| | dimer | | |
| Phase II | 3,3-Diethyl-N-[1(R)-(3,4- | L-694458; | EP 0595557; GB 2280673; |
| | methylenedioxyphenyl)butyl]-2(S)-[4- | DMP-777 | WO 1995024207; WO 1997016448; |
| | (4-methylpiperazin-1- | | WO 2000012474 |
| | ylcarbonyl)phenoxy]-4-oxoazetidine- | | |
| | 1-carboxamide | | |
| Phase II | Second-generation primatized anti- | IDEC-151; | |
| | CD4 antibody; Immunoglobulin G4 | SB-217969, | |
| | anti-(human CD4 [antigen]) (human- | Clenoliximab; | |
| | Macaca monoclonal CE9gamma4PE | Lenoliximab | |
| | gamma4-chain), disulfide with | | |
| | human-Macaca monoclonal | | |
| | CE9gamma4PE kappa-chain, dimer | | |
| Phase II | N-[2(R)-Ethoxy-5- | HMR-3480; | WO 1997022619; WO 1999052935; |
| | oxotetrahydrofuran-3(S)-yl]-9(S)-(1- | VX-740, | WO 2000010979; WO 2000042061; |
| | isoquinolinylcarboxamido)-6,10- | Pralnacasan | WO 2004026406; WO 2005053665; |
| | dioxooctahydro-6H-pyridazino[1,2- | | WO 2005115362; WO 2005117846; |
| | a][1,2]diazepine-1(S)-carboxamide | | WO 2007042160 |
| Phase II | Combination of three peptides derived | IR-501, RAVAX | |
| | from T-cell receptors | | |
| | (Vbeta3,Vbeta14,Vbeta17) in | | |
| | incomplete Freund's adjuvant (IFA) | | |
| Phase II | 4-(Methylsulfanyl)phenylsulfanylmethanediphosphonic | TRK-530 | JP 2003238415; U.S. Pat. No. 6,555,529; |
| | acid disodium | | U.S. Pat. No. 6,579,860; WO 1993005052; |
| | salt | | WO 1994019359 |
| Phase II | Fully human monoclonal antibody | ABX-IL-8 | WO 2003080117 |
| | which binds to interleukin-8 | | |
| Phase II | N-(Ethoxycarbonyl)-4-[3-[4-[1-(4- | BIIL-284, | DE 4424713; WO 2002055065; |
| | hydroxyphenyl)-1- | Amelubant | WO 2003007922; WO 2004047824; |
| | methylethyl]phenoxymethyl]benzyloxy]- | | WO 2005041855 |
| | benzenecarboximidamide | | |
| Phase II | S-[2-(1-Iminoethylamino)ethyl]-L- | GW-274150; | WO 1998030537; WO 2003030935 |
| | homocysteine; 2(S)-Amino-7-(1- | 274150 | |
| | iminoethylamino)-5-thiaheptanoic | | |
| | acid | | |
| Phase II | Standardized devil's claw | WS-1531 | WO 1997034565 |
| | (Harpagophytum procumbens) extract | | |
| | rich in harpagoside | | |
| Phase II | A specific myxoma virus 55-kDa | SERP1; SERP-1; | US 2003171263; US 2006122115; |
| | secreted glycoprotein with homology | VT-111 | US 2008070841; U.S. Pat. No. 5,917,014; |
| | to serine protease inhibitors | | WO 1995027503; WO 2001039790; |
| | | | WO 2002026245 |
| Phase II | N-[4-(2,4-diamino-6- | AMT; NSC-739, | US 2005209239; WO 2009126274 |
| | pteridinylmethylamino)benzolyl]-L- | Aminopterin, | |
| | glutamic acid | Aminotrexate | |
| Phase II | 5-(2,6-Dichlorophenyl)-2-(2,4- | VX-745 | WO 1998027098; WO 2005091891 |
| | difluorophenylsulfanyl)-6H- | | |
| | pyrimido[3,4-b]pyridazin-6-one | | |
| Phase II | 4′-Thio-beta-D- | VP-700; SR-9025; | |
| | arabinofuranosylcytosine; 4-Amino-1- | OSI-7836; 4′-Thio- | |
| | (4-thio-beta-D-arabinofuranosyl)- | ara-C; T-araC; | |
| | 2(1H)-pyrimidinone; 1-(4-Thio-beta- | VP-701; GS-7836, | |
| | D-arabinofuranosyl)cytosine | Thiarabine | |
| Phase II | (3S,9S,12S,15R,18S)-3-(N-Acetyl-L- | PMX-53; AcF- | WO 1999000406; WO 2003033528; |
| | phenylalanylamino)-15- | [OP(D-Cha)WR] | WO 2003086448; WO 2004035078; |
| | (cyclohexylmethyl)-9-(3- | | WO 2004035079; WO 2004035080; |
| | guanidinopropyl)-12-(1H-indol-3- | | WO 2004100975; WO 2004103392; |
| | ylmethyl)-1,7,10,13,16- | | WO 2005092366; WO 2006099330; |
| | pentaazabicyclo[16.3.0]heneicosane- | | WO 2009105217 |
| | 2,8,11,14,17-pentaone; N-Acetyl-L- | | |
| | phenylalanyl-L-ornithyl-L-prolyl-3- | | |
| | cyclohexyl-D-alanyl-L-tryptophyl-L- | | |
| | arginine N-5.2-C-1.6-lactam | | |
| Phase II | 2-Methoxyestra-1,3,5(10)-triene- | 2-MeOE2; 2-ME; | US 2004053906; US 2008131484; |
| | 3,17beta-diol | NSC-659853; | US 2008220075; US 2008279936; |
| | | 2ME2, 2- | US 2008292679; US 2008293683; |
| | | Methoxyestradiol, | WO 2001014405; WO 2002003979; |
| | | Panzem NCD; | WO 2003015704; WO 2003073985; |
| | | Panzem; | WO 2003080027; WO 2005051357; |
| | | PulmoLAR | WO 2005082458; WO 2005110366; |
| | | | WO 2005110462; WO 2006032026; |
| | | | WO 2006058298; WO 2007059111; |
| | | | WO 2007109312; WO 2008094665 |
| Phase II | Recombinant (E. coli) form of the | PEG-sTNF-RI, | US 2008124383; WO 2004000211; |
| | high-affinity p55 soluble tumor | Pegsunercept; | WO 2005079794; WO 2005079796 |
| | necrosis factor receptor type I (sTNF- | Pegylated soluble | |
| | RI) to which a 30-kD polyethylene | tumor necrosis | |
| | glycol (PEG) molecule is attached; | factor receptor type I | |
| | Pegylated (30 kD) L-methionyl-1-105- | | |
| | tumor necrosis factor receptor p55 | | |
| | (human) | | |
| Phase II | (PB-7-11-2344′3′)- | M-40403; SC- | EP 1420022; US 2002072512; |
| | Dichloro[(4aR,13aR,17aR,21aR)- | 72325, Imisopasem | WO 1998058636; WO 2002053142; |
| | 11,7-nitrilo- | manganese | WO 2002058686; WO 2003051458; |
| | 2,3,4,4a,5,6,7,12,13,13a,14,15,16,17,17a, | | WO 2005041885; WO 2005041894; |
| | 18,19,20,21,21a-eicosahydro-1H- | | WO 2005042718; WO 2005060974; |
| | dibenzo[b,h][1,4,7,10]tetraazacycloheptadecine- | | WO 2006078713; WO 2006083508; |
| | kappaN5,kappaN13,kappaN18,kappa | | WO 2008045559 |
| | N21,kappaN22]manganese | | |
| Phase II | Complex of the solubilized class II | AG-4263, | |
| | MHC molecule HLA-DRB1*0401 | AnergiX-RA; | |
| | together with a specific 13-mer | RA-AnergiX | |
| | peptide (CDP263) derived from the | | |
| | human cartilage glycoprotein 39 | | |
| | (HCgp-39) | | |
| Phase II | 2-Cyano-3,12-dioxoolean-1,9(11)- | CDDO-Me; | WO 1999065478; WO 2005082458; |
| | dien-28-oic acid methyl ester; | RTA-402; | WO 2008016095; WO 2008064132; |
| | (6aR,6bS,8aR,12aS,14aR,14bS)-11- | TP-155C; | WO 2008111497; WO 2009023232; |
| | Cyano-2,2,6a,6b,9,9,12a-heptamethyl- | Methyl-CDDO; | WO 2009089545 |
| | 10,14-dioxo- | NSC-713200; | |
| | 1,3,4,5,6,6a,6b,7,8,8a,9,10,12a,14,14a, | BARD, | |
| | 14b-hexadecahydropicene-4a(2H)- | Bardoxolone | |
| | carboxylic acid methyl ester | methyl | |
| Phase II | | KSB-302; | |
| | | CBF-BS2 | |
| Phase II | N-[4-(N- | ITF-2357, | U.S. Pat. No. 6,034,096; WO 2004064824; |
| | Hydroxycarbamoyl)phenyl]carbamic | Givinostat | WO 2004065355 |
| | acid 6- | hydrochloride | |
| | (diethylaminomethyl)naphthalen-2- | | |
| | ylmethyl ester hydrochloride; 4-[6- | | |
| | (Diethylaminomethyl)naphthalen-2- | | |
| | ylmethoxycarbonylamino]benzohydro | | |
| | xamic acid hydrochloride | | |
| Phase II | Immunoglobulin G1, anti-(human | HuZAF, | EP 1336654; WO 2000032634; |
| | interferon gamma) (human-mouse | Fontolizumab; | WO 2003097082; WO 2008156807 |
| | monoclonal HuZAF gamma1-chain), | SMART anti- | |
| | disulfide with human-mouse | interferon gamma | |
| | monoclonal HuZAF light chain, dimer | antibody | |
| Phase II | N-[3-tert-Butyl-1-(4-methylphenyl)- | BIRB-796; BIRB- | WO 2000043384; WO 2001004115; |
| | 1H-pyrazol-5-yl]-N′-[4-[2-(4- | 0796; BIBR-796; | WO 2002007772; WO 2003005999; |
| | morpholinyl)ethoxy]naphthalen-1- | BIRB-796 BS, | WO 2003015828; WO 2003049742; |
| | yl]urea | Doramapimod | WO 2003068223; WO 2003084503; |
| | | | WO 2005009367; WO 2005018624; |
| | | | WO 2005058308; WO 2005063715; |
| | | | WO 2005091891; WO 2005110455; |
| | | | WO 2006127678 |
| Phase II | 4-[4-Chloro-5-(3-fluoro-4- | UR-8880, | WO 2000023426; WO 2003016285; |
| | methoxyphenyl)-1H-imidazol-1- | Cimicoxib | WO 2005007106; WO 2005007156; |
| | yl]benzenesulfonamide | | WO 2005037193; WO 2006077334 |
| Phase II | Anti-interferon gamma antibody | A-INF-gamma; | US 2004086508; U.S. Pat. No. 5,626,843; |
| | | Anti-IFN-gamma | U.S. Pat. No. 5,888,511; U.S. Pat. No. 6,333,032; |
| | | MAb | WO 2005037868; WO 2005058237; |
| | | | WO 2006002057; WO 2006002058; |
| | | | WO 2006044263 |
| Phase II | 6-[1-(2,6-Difluorophenyl)ureido]-2- | KVK-702; VX-702 | WO 2004072038; WO 2007103468 |
| | (2,4-difluorophenyl)pyridine-3- | | |
| | carboxamide | | |
| Phase II | | IPL-512602 | |
| Phase II | Recombinant nonglycosylated human | MM-093; rhAFP; | WO 2007126847; WO 2009124056 |
| | alpha-fetoprotein (rhAFP) | ABI-001 | |
| Phase II | beta-Lymphotoxin receptor (human | LTbetaR-Fc; | WO 1997003687; WO 1999038525; |
| | extracellular domain-containing | LTbetaR-Ig; | WO 2000021558; WO 2000036092; |
| | fragment) fusion protein with | LTBR-Fc; | WO 2006135660; WO 2007122402 |
| | immunoglobulin G1 (human gamma1- | LTBRIg; BG-9924, | |
| | chain Fc fragment); Human tumor | Baminercept alfa | |
| | necrosis factor receptor superfamily | | |
| | member 3 (lymphotoxin-beta receptor, | | |
| | TNF C receptor)-(2-195)-peptide | | |
| | (fragment of extracellular domain) | | |
| | fusion protein with human | | |
| | immunoglobulin heavy constant | | |
| | gamma-chain Fc fragment [227 | | |
| | residues, hinge (195-205) des-(1-4), | | |
| | C5 > V, CH2 (206-315), CH3 (316-421) | | |
| | des-K107]dimer (201-201′:204-204′)- | | |
| | bisdisulfide, glycosolated | | |
| Phase II | | PW-9101; AD-121 | |
| Phase II | Sterilized immune globulin product | IgPO, Human | WO 2006036213; WO 2009054226 |
| | prepared from pooled normal human | gammaglobulin, | |
| | donor plasma that consists primarily | Oralgam | |
| | of IgG | | |
| Phase II | Modified recombinant human | AMG-719 | |
| | interleukin-1 receptor antagonist | | |
| Phase II | 7beta-Hydroxyepiandrosterone; (+)- | HF-0220, 7beta- | WO 2002000225; WO 2008065408 |
| | 3beta,7beta-Dihydroxyandrostan-17- | Hydroxyepiandrosterone; | |
| | one | 7beta-OH- | |
| | | EPIA | |
| Phase II | N-[2-(1-Adamantyl)ethyl]-N-pentyl- | DE-096; SA-13353 | JP 2009062284; WO 2001092229; |
| | N′-[3-(4-pyridyl)propyl]urea | | WO 2003045367; WO 2005102331; |
| | | | WO 2005102332; WO 2006035759; |
| | | | WO 2006035760; WO 2006043518; |
| | | | WO 2009051151 |
| Phase II | | SSR-150106 | |
| Phase II | 2(R)-[3(R)-Amino-3-[4-(2- | DPC-333; | WO 2003082287; WO 2009132050 |
| | methylquinolin-4-ylmethoxy)phenyl]- | BMS-561392 | |
| | 2-oxopyrrolidin-1-yl]-4- | | |
| | methylpentanehydroxamic acid | | |
| Phase II | N,5-Diethyl-4-hydroxy-1-methyl-2- | ABR-215757; | WO 1999055678; WO 2001030758; |
| | oxo-N-phenyl-1,2-dihydroquinoline- | 57-57, Paquinimod | WO 2005074899 |
| | 3-carboxamide | | |
| Phase II | | T-487 | |
| Phase II | Humanized monoclonal antibody | MLN-1202 | |
| | against CCR2 | | |
| Phase II | N-(2,6-Difluorobenzoyl)-4-[4- | T-0047; 683699; | JP 2003321358; WO 2002018320; |
| | (ethoxymethyl)-2,6- | SB-683699, | WO 2003072536 |
| | dimethoxyphenyl]-L-phenylalanine | Firategrast | |
| Phase II | 8-(2,6-Difluorophenyl)-4-(4-fluoro-2- | SB-681323-T | WO 2002059083; WO 2006127678; |
| | methylphenyl)-2-[2-hydroxy-1- | (p-toluenesulfonate | WO 2007059500; WO 2007147104 |
| | (hydroxymethyl)ethylamino]pyrido[2, | salt); SB-681323; | |
| | 3-d]pyrimidin-7(8H)-one | 681323, | |
| | | Dilmapimod | |
| Phase II | (−)-(S)-8-[4-(2-Butoxyethoxy)phenyl]- | TBR-652; | EP 1484322; WO 2003014105; |
| | 1-isobutyl-N-[4-(1-propyl-1H- | TAK-652 | WO 2005116013 |
| | imidazol-5-ylmethylsulfinyl)phenyl]- | | |
| | 1,2,3,4-tetrahydro-1-benzazocine-5- | | |
| | carboxamide methanesulfonate | | |
| Phase II | 3-Methylbenzaldehyde [6-(4- | STA-5326 | WO 2005000404; WO 2005112938; |
| | morpholinyl)-2-[2-(2- | mesylate, Apilimod | WO 2006060194; WO 2006128129; |
| | pyridyl)ethoxy]pyrimidin-4- | mesylate | WO 2006128172; WO 2007100759 |
| | yl]hydrazone bis(methanesulfonate) | | |
| Phase II | Immunoglobulin G1, anti-(human B- | hA20; IMMU-106, | US 2009169550; WO 2003068821; |
| | lymphocyte antigen CD20 | Veltuzumab | WO 2004058298 |
| | (Membrane-spanning 4-domains | | |
| | subfamily A member 1, Leu-16, | | |
| | Bp35)); [218-arginine,360-glutamic | | |
| | acid,362-methionine]humanized | | |
| | mouse monoclonal hA20 gamma1 | | |
| | heavy chain (224-213′)-disulfide with | | |
| | humanized mouse monoclonal hA20 | | |
| | kappa light chain (230-230″:233-233″)- | | |
| | bisdisulfide dimer; | | |
| | Immunoglobulin G1, anti-[Homo | | |
| | sapiens CD20 (MS4A1, membrane- | | |
| | spanning 4-domains subfamily A | | |
| | member 1, B lymphocyte surface | | |
| | antigen B1, Leu-16, Bp35)] | | |
| | humanized monoclonal IMMU-106 | | |
| | (or hA20); gamma1 heavy chain | | |
| | [humanized VH (Homo sapiens | | |
| | FR/Mus musculus CDR) [8.8.14]- | | |
| | Homo sapiens IGHG1*03] (224-213′)- | | |
| | disulfide with kappa light chain | | |
| | [humanized V-KAPPA (Homo | | |
| | sapiens FR/Mus musculus CDR) | | |
| | [5.3.9]-Homo sapiens IGKC*01]; | | |
| | (230-230″:233-233″)-bisdisulfide | | |
| | dimer; Immunoglobulin G1, anti- | | |
| | (human CD20 (antigen)) (human- | | |
| | mouse monoclonal hA20 heavy | | |
| | chain), disulfide with human-mouse | | |
| | monoclonal hA20 kappa-chain, dimer | | |
| Phase II | 3-[5-[4-(Cyclopentyloxy)-2- | R-7277; T-5224 | WO 2003042150; WO 2007097279; |
| | hydroxybenzoyl]-2-(3-hydroxy-1,2- | | WO 2007138996; WO 2007138997; |
| | benzisoxazol-6- | | WO 2009040952; WO 2009069643; |
| | ylmethoxy)phenyl]propionic acid | | WO 2009119652; WO 2009131098 |
| Phase II | 2-(3-Fluoro-4-hydroxyphenyl)-7- | ERB-041; | US 2008132554; US 2008139633; |
| | vinylbenzoxazol-5-ol | WAY-202041, | US 2008175900; US 2008175901; |
| | | Prinaberel | US 2008176914; US 2008182872; |
| | | | US 2008241234; WO 2003050095; |
| | | | WO 2004062653; WO 2006060384; |
| | | | WO 2006060532; WO 2006060542; |
| | | | WO 2006096591; WO 2008064217 |
| Phase II | | K-832 | |
| Phase II | 7-[3(R)-Hydroxy-4(R)- | RO-5092888; | WO 2004018496; WO 2004069856 |
| | (hydroxymethyl)pyrrolidin-1- | BCX-4208; | |
| | ylmethyl]-5H-pyrrolo[3,2- | R-3421, DADMe- | |
| | d]pyrimidin-4-ol | Immucillin-H | |
| Phase II | | INCB-3284; | |
| | | INCB-003284 | |
| Phase II | Combination of amoxapine and low | CRx-119, | US 2004110734; WO 2003006026; |
| | dose prednisolone | Amoxapine/ | WO 2005030132 |
| | | prednisolone | |
| Phase II | Combination of prednisolone and | CRx-139, | |
| | paroxetine | Prednisolone/ | |
| | | paroxetine | |
| Phase II | Combination of dipyridamole and low | CRx-102, | US 2006234991; US 2007010502; |
| | dose prednisolone | Dipyridamole/ | US 2009075955; WO 2003030823; |
| | | prednisolone, | WO 2005030132; WO 2007089617; |
| | | Synavive | WO 2007139753 |
| Phase II | | SC-12267; | WO 2003006425 |
| | | 4SC-101 | |
| Phase II | | MLN-3897; | |
| | | AVE-9897 | |
| Phase II | | AV-1142742, | WO 2004081011 |
| | | Rhudex | |
| Phase II | 4-[3-[5-Chloro-2-[2-(3,4- | PLA-902, | US 2003149029; US 2003166649; |
| | dichlorobenzylsulfonamido)ethyl]-1- | Efipladib | US 2004082785; U.S. Pat. No. 6,635,771; |
| | (1,1-diphenylmethyl)-1H-indol-3- | | U.S. Pat. No. 6,797,708; WO 2003048122; |
| | yl]propyl]benzoic acid | | WO 2006023611; WO 2007140317 |
| Phase II | Anti-tumor necrosis factor (TNF)- | PN-0621; Dom- | |
| | alpha Domain Antibody (dAb) | 0200; ART-621; | |
| | | CEP-37247 | |
| Phase II | | C-4462 | |
| Phase II | Fully human monoclonal antibody | AMG-108 | |
| | against IL-1R type I | | |
| Phase II | Small modular | TRU-015; | US 2005175614; US 2009169550; |
| | immunopharmaceutical product | CytoxB20G | WO 2005037989; WO 2007014238; |
| | (SMIP) consisting of a single chain | | WO 2007014278 |
| | construct of a modified single chain | | |
| | Fv linked to modified human IgG1 | | |
| | hinge, CH2, and CH3 domains that | | |
| | binds to CD20 | | |
| Phase II | Polyherbal drug | IRA-01, Insix | |
| Phase II | Fully human anti-IP-10 (anti- | MDX-1100 | WO 2005011605; WO 2005023201 |
| | CXCL10) monoclonal antibody | | |
| Phase II | Humanized anti-CD6 monoclonal | T1h; h-T1 | EP 0807125; WO 2009113083 |
| | antibody | | |
| Phase II | | YS-IL6; YSIL-6 | |
| Phase II | Therapeutic vaccine composed of | Qbeta-C-TNF(4- | WO 2005117963 |
| | TNF-alpha-derived peptide chemically | 23); CYT-007- | |
| | coupled to the virus-like particle Qb | TNFQb | |
| Phase II | 4-[4-(4-Hydroxy-2- | TMI-005, | US 2005272725; US 2005272928; |
| | butynyloxy)phenylsulfonyl]-2,2- | Apratastat | U.S. Pat. No. 6,225,311; WO 2000044709; |
| | dimethylthiomorpholine-3(S)- | | WO 2002083112; WO 2007107663 |
| | carbohydroxamic acid; N-Hydroxy-4- | | |
| | [4-(4-hydroxy-2-butynyloxy)- | | |
| | phenylsulfonyl]-2,2-dimethyl-3- | | |
| | thiomorpholinecarboxamide | | |
| Phase II | N-[4-[2-(2,4-Diaminoquinazolin-6- | MobileTrex; | US 2005020833; US 2009253720; |
| | yl)ethyl]benzoyl]-4- | CH-1504; | U.S. Pat. No. 5,912,251; WO 2004045500; |
| | methyleneglutamic acid | TRIDAM; M-trex | WO 2006029385 |
| Phase II | | NF-kB Decoy, | WO 2009119836 |
| | | NF-kappaB Decoy, | |
| | | Avrina | |
| Phase II | (+)-N-[2-[1(S)-(3-Ethoxy-4- | CC-10004, | WO 2003080049; WO 2006065814; |
| | methoxyphenyl)-2- | Apremilast | WO 2009120167 |
| | (methylsulfonyl)ethyl]-1,3-dioxo-2,3- | | |
| | dihydro-1H-isoindol-4-yl]acetamide | | |
| Phase II | 3-Methylbenzaldehyde [6-(4- | STA-5326, | WO 2005000404; WO 2006060194; |
| | morpholinyl)-2-[2-(2- | Apilimod | WO 2006128129; WO 2006128172; |
| | pyridyl)ethoxy]pyrimidin-4- | | WO 2007100759; WO 2009100406 |
| | yl]hydrazone | | |
| Phase II | 6-[5-(N-Cyclopropylcarbamoyl)-3- | GW-856553X; | WO 2003068747; WO 2006127678; |
| | fluoro-2-methylphenyl]-N-(2,2- | SB-856553; | WO 2007144390 |
| | dimethylpropyl)pyridine-3- | GW-856553; | |
| | carboxamide | 856553, | |
| | | Losmapimod | |
| Phase II | 6-[5-Fluoro-2-(3,4,5- | NSC-742317; | WO 2005012294; WO 2005013996; |
| | trimethoxyphenylamino)pyrimidin-4- | R-406 | WO 2005016893; WO 2006135915; |
| | ylamino]-2,2-dimethyl-3,4-dihydro- | | WO 2007053844 |
| | 2H-pyrido[3,2-b][1,4]oxazin-3-one | | |
| | benzenesulfonate | | |
| Phase II | 2-[6-Chloro-5-[4-(4-fluorobenzyl)- | SCIO-469, | U.S. Pat. No. 6,867,209; WO 2000071535; |
| | 2(R),5(S)-dimethylpiperazin-1- | Talmapimod | WO 2003041644; WO 2004019873; |
| | ylcarbonyl]-1-methyl-1H-indol-3-yl]- | | WO 2004021988; WO 2005032551; |
| | N,N-dimethyl-2-oxoacetamide | | WO 2006055302; WO 2007005863; |
| | | | WO 2008024391 |
| Phase II | 20-Mer double-stranded | NFkB decoy oligo | JP 2005314381; JP 2006089475; |
| | phosphorothioate decoy | | US 2006153847; WO 1996035430; |
| | oligodeoxynucleotide directed against | | WO 2005004914; WO 2006064886; |
| | the nuclear factor-kappa B (NF- | | WO 2006075776; WO 2006122242; |
| | kappaB)-binding cis-elements, whose | | WO 2006132204 |
| | complementary sequences are: 5′- | | |
| | CCTTGAAGGGATTTCCCTCC-3′ | | |
| | and 3′- | | |
| | GGAACTTCCCTAAAGGGAGG-5′ | | |
| Phase II | 4-Butyl-4-hydroxy-1-(4- | AV-1101; | WO 2001000585; WO 2005107748 |
| | hydroxyphenyl)-2- | 4OH-OPB, | |
| | phenylpyrazolidine-3,5-dione | 4-Hydroxyoxyphen | |
| | | butazone | |
| Phase II | | PMI-001 | |
| Phase II | | KC-706 | |
| Phase II | | RO-496-4913 | |
| Phase II | 1-[1(S)-Isopropyl-3(R)-[3(S)- | MK-0812 | WO 2005044264 |
| | methoxytetrahydropyran-4(S)- | | |
| | ylamino]cyclopentyl]-1-[3- | | |
| | (trifluoromethyl)-5,6,7,8-tetrahydro- | | |
| | 1,6-naphthyridin-6-yl]methanone | | |
| | succinate | | |
| Phase II | Humanized IgG2 monoclonal | XMA-005.2; | WO 2007002261; WO 2009086003 |
| | antibody against human interleukin- | XOMA-052 | |
| | 1beta | | |
| Phase II | Phosphoric acid 6-[5-fluoro-2-(3,4,5- | NSC-745942; | WO 2006078846; WO 2008061201; |
| | trimethoxyphenylamino)pyrimidin-4- | R-788; R-935788, | WO 2008064274; WO 2009061909 |
| | ylamino]-2,2-dimethyl-3-oxo-3,4- | Fostamatinib | |
| | dihydro-2H-pyrido[3,2-b][1,4]oxazin- | disodium; | |
| | 4-ylmethyl monoester disodium salt | Tamatinib fosdium | |
| | hexahydrate; 6-[5-fluoro-2-(3,4,5- | | |
| | trimethoxyphenylamino)pyrimidin-4- | | |
| | ylamino]-2,2-dimethyl-4- | | |
| | (phosphonooxymethyl)-3,4-dihydro- | | |
| | 2H-pyrido[3,2-b][1,4]oxazin-3-one | | |
| | disodium salt hexahydrate | | |
| Phase II | N-[3-[3-Cyclopropyl-5-(2-fluoro-4- | GSK-1120212 ( ); | WO 2005121142 |
| | iodophenylamino)-6,8-dimethyl-2,4,7- | GSK-1120212B; | |
| | trioxo-1,2,3,4,6,7- | 1120212 | |
| | hexahydropyrido[4,3-d]pyrimidin-1- | | |
| | yl]phenyl]acetamide dimethyl | | |
| | sulfoxide solvate | | |
| Phase II | 2-(9-Chloro-2,3-dimethyl-6H- | Rob-803, | WO 2005123741 |
| | indolo[2,3-b]quinoxalin-6-yl)-N-[2- | Rabeximod | |
| | (dimethylamino)ethyl]acetamide | | |
| Phase II | Polymerized porcine type I collagen- | Clg-PVP, | WO 2005115443 |
| | polyvinylpyrrolidone (PVP) | Collagen-PVP | |
| Phase II | Recombinant human heat shock | Hsp10; Cpn-10, | WO 2004041300; WO 2005067959; |
| | protein 10 (HSPE1) that differs from | Chaperonin-10; | WO 2007006095; WO 2007098557 |
| | the native protein by the addition of | Heat Shock Protein | |
| | one alanine residue; Chaperonin-10 | 10, XToll | |
| Phase II | | ALS-00T2-0501; | |
| | | ALS-00T2 | |
| Phase II | Pregn-5-en-20-yne- | HE-3286, Triolex | US 2008015174; WO 2008039566; |
| | 3beta, 7beta, 17alpha-triol | | WO 2009124300 |
| Phase II | | LX-3305; LX-2931 | |
| Phase II | | PG-760564 | |
| Phase II | | ARRY-371797; | |
| | | ARRY-797 | |
| Phase II | Humaneered(TM) monoclonal | KB-003 | US 2009181020; WO 2008063898; |
| | antibody targeted to granulocyte | | WO 2008064321 |
| | monocyte colony stimulating factor | | |
| | (GM-CSF) | | |
| Phase II | Anti-TNFalpha nanobody | anti-TNFalpha | WO 2006122786; WO 2009080764 |
| | | Nanobody | |
| Phase II | | RWJ-445380 | |
| Phase II | | PH-797804 | |
| Phase II | Immunoglobulin G1-lambda, anti- | ILV-094, | |
| | [Homo sapiens interleukin 22 (IL22, | Fezakinumab | |
| | IL-22, ILTIF, IL-TIF)], Homo sapiens | | |
| | monoclonal antibody; gamma1 heavy | | |
| | chain (1-450) [Homo sapiens VH | | |
| | (IGHV1-2*02 (91.80%)-(IGHD)- | | |
| | IGHJ2*01) [8.8.14] (1-121)- | | |
| | IGHG1*03 CH1 R120 > K, CH3 | | |
| | K130 > del (122-450)], (224-216′)- | | |
| | disulfide with lambda light chain (1′-217′) | | |
| | [Homo sapiens V-LAMBDA | | |
| | (IGLV1-40*01 (96.00%)-IGLJ2*01 | | |
| | K123 > Q) [9.3.11] (1′-111′)- | | |
| | IGLC2*01 (112′-217′)]; (230-230″: | | |
| | 233-233″)-bisdisulfide dimer; | | |
| | Immunoglobulin G1, anti-(human | | |
| | interleukin 22) (human monoclonal | | |
| | heavy chain), disulfide with human | | |
| | monoclonal lamda-chain, dimer; | | |
| | Immunoglobulin G1, anti- | | |
| | (human/Macaca irus/Rattus/Mus | | |
| | musculus interleukin 22) (human | | |
| | monoclonal heavy chain), disulfide | | |
| | with human monoclonal lamda-chain, | | |
| | dimer | | |
| Phase II | | PS-540446; | |
| | | BMS-582949 | |
| Phase II | | 315234; | |
| | | GSK-3152314A | |
| Phase II | | CG-100649 | |
| Phase II | | ALD-518 | |
| Phase II | | SBI-087 | |
| Phase II | | INCB-28050; | |
| | | INCB-028050 | |
| Phase II | | PF-00251802; | |
| | | PF-251802 | |
| Phase II | | PF-04171327; | |
| | | PF-4171327 | |
| Phase II | Fully human monoclonal antibody | AMG-827 | US 2009074758 |
| | targeting the IL-17 receptor | | |
| Phase II | Liposome formulation of prednisolone | Nanocort | |
| Phase II | Human antibody targeting BAFF | LY-2127399 | |
| Phase II | | NPS-31807 | |
| Phase II | | CNTO-136 | |
| Phase II | Humanized monoclonal antibody | MTRX-1011A; | |
| | targeting CD4 | RO-4989991; | |
| | | huMAb OX40L | |
| Phase II | | GSK-706769 | |
| Phase II | Antibody targeting IL-17 | LY-2439821 | |
| Phase II | Antibody targeting IL-1beta | LY-2189102 | |
| Phase II | | AK-106-001616 | |
| Phase II | Synovial fibroblasts genetically | | WO 1996022793 |
| | modified with the amphotropic | | |
| | retrovirus, MFG-IRAP, which carries | | |
| | the full-length coding sequence for | | |
| | human IL-1Ra under the | | |
| | transcriptional regulation of the viral | | |
| | long terminal repeat | | |
| Phase II | | Excellair | WO 2005007623 |
| Phase II | | JNJ-38518168 | |
| Phase II/III | (Z)-2-Amino-5-(3,5-di-tert-butyl-4- | PD-136095-0073; | EP 0449216; WO 1998002160 |
| | hydroxybenzylidene)thiazol-4(5H)- | CI-1004, | |
| | one methanesulfonate | Darbufelone | |
| | | mesilate | |
| Phase II/III | Human transmembrane activator and | TACI-Ig; sTACI; | WO 2002094852; WO 2004033486; |
| | CAML interactor (TACI)- | TACI-Fc5, | WO 2007019618; WO 2007134326; |
| | Immunoglobulin G1 Fc domain fusion | Atacicept | WO 2008025747; WO 2008025748; |
| | protein (Fc5); 1-81-TACI (human) | | WO 2008119042; WO 2008157369; |
| | fusion protein with 82-313-modified | | WO 2009132058 |
| | immunoglobulin G1 (human gamma1- | | |
| | chain Fc fragment), dimer. Monomers | | |
| | are covalently linked by disulfide | | |
| | bridges at cysteines 92 and 95 of each | | |
| | monomer; [86-Serine,101-glutamic | | |
| | acid,196-serine,197-serine, 222- | | |
| | aspartic acid, 224-leucine][human | | |
| | tumor necrosis factor receptor | | |
| | superfamily member 13B-(30-110)- | | |
| | peptide (TACI fragment containing | | |
| | TNFR-Cys 1 and TNFR-Cys 2) fusion | | |
| | protein with human immunogobulin | | |
| | G1-(232 C-terminal residues)-peptide | | |
| | (gamma1-chain Fc fragment), (92-92′: | | |
| | 95-95′)-bisdisulfide dimer; Soluble | | |
| | form of the TACI receptor | | |
| | (transmembrane activator and | | |
| | calcium-modulating and cyclophyllin | | |
| | ligand [CAML] interactor), a cell | | |
| | surface receptor found on B- | | |
| | lymphocytes and activated T-cells, | | |
| | made by fusing the TACI extracellular | | |
| | domain to the Fc portion of human | | |
| | IgG1 | | |
| Phase II/III | | LAS-34475 | |
| Phase III | 3-O-[3-(Dimethylamino)propyl]-1,2- | SM-1213, | DE 2455026 |
| | O-(1-methylethylidene)-alpha-D- | Amiprilose, | |
| | glucofuranose; 1,2-O-Isopropylidene- | Therafectin | |
| | 3-O-[3′-(N,N-dimethylamino)propyl]- | | |
| | D-glucofuranose | | |
| Phase III | (E,E)-1,7-Bis(4-hydroxy-3- | NSC-32982, | EP 2070545; JP 2003055202; |
| | methoxyphenyl)-1,6-heptadiene-3,5- | Curcumin; | JP 2003128539; JP 2006151878; |
| | dione | Curcumin I; | JP 2008201768; JP 2009023954; |
| | | Diferuloylmethane | JP 2009227609; JP 2009249370; |
| | | | US 2004167217; US 2008075671; |
| | | | US 2008260695; U.S. Pat. No. 6,306,383; |
| | | | U.S. Pat. No. 6,673,843; WO 1994004139; |
| | | | WO 2001019158; WO 2003007975; |
| | | | WO 2003088986; WO 2003090681; |
| | | | WO 2004000229; WO 2004080396; |
| | | | WO 2005020908; WO 2005020958; |
| | | | WO 2005077394; WO 2005079856; |
| | | | WO 2005113069; WO 2006087759; |
| | | | WO 2006089894; WO 2007101551; |
| | | | WO 2007110168; WO 2008016095; |
| | | | WO 2008043157; WO 2008103346; |
| | | | WO 2008131354; WO 2009003147; |
| | | | WO 2009061152; WO 2009073050; |
| | | | WO 2009080842; WO 2009080850; |
| | | | WO 2009101263; WO 2009105278; |
| | | | WO 2009114525; WO 2009120815; |
| | | | WO 2009126950 |
| Phase III | Mixture of two semisynthetic lignan | CPH-82, | |
| | glycosides from the Podophyllum | Reumacon | |
| | plant | | |
| Phase III | 4-(2′,4′-Difluorobiphenylyl)-4-oxo-2- | VUFB-16066, | JP 1986065842 |
| | methylbutanoic acid; 3-[4-(2,4- | Flobufen | |
| | Difluorophenyl)benzoyl]-2- | | |
| | methylpropionic acid; gamma-Oxo- | | |
| | (2′,4′-difluoro-[1,1′]biphenyl-4-yl)-2- | | |
| | methylbutanoic acid; 2′,4′-Difluoro- | | |
| | alpha-methyl-gamma-oxo-[1,1′- | | |
| | biphenyl]-4-butanoic acid | | |
| Phase III | (±)-2-(Acetylsulfanylmethyl)-4-(4- | KE-298, | EP 0164101; JP 1987132825 |
| | methylphenyl)-4-oxobutyric acid; (±)- | Esonarimod | |
| | 2-(Acetylsulfanylmethyl)-3-(4- | | |
| | methylbenzoyl)propionic acid | | |
| Phase III | 3-(Formylamino)-7- | T-614, Iguratimod, | JP 1990268178; JP 2001055331; |
| | (methylsulfonamido)-6-phenoxy-4H- | Colvet; Careram; | JP 2007224021; WO 1994023714; |
| | 1-benzopyran-4-one | Kolbet | WO 2005094788 |
| Phase III | Pyridoxylated hemoglobin- | VTR-PHP; | |
| | polyoxyethylene conjugate | PHP-HT; PHP; | |
| | | PHP-CT | |
| Phase III | Immunoglobulin G1, anti-(human | ABT-874; J-695; | WO 2000056772; WO 2008121301; |
| | interleukin-12 subunit beta (IL-12 | A-796874.0; | WO 2009073569; WO 2009117289 |
| | subunit p40, CLMF p40 or NKSF2)); | WAY-165772; | |
| | human monoclonal gamma1 heavy | LU-415977; | |
| | chain (218-216′)-disulfide with human | BSF-415977, | |
| | monoclonal lamda light chain, dimer | Briakinumab | |
| | (224-224″:227-227″)-bisdisulfide; | | |
| | Immunoglobulin G1-lambda, anti- | | |
| | [Homo sapiens interleukin 12 beta | | |
| | subunit (IL12B, IL-12B, IL12 p40, | | |
| | NKSF2, CMLF p40)], Homo sapiens | | |
| | monoclonal antibody; gamma1 heavy | | |
| | chain (1-445) [Homo sapiens VH | | |
| | (IGHV3-30*02(99.00%)-(IGHD)- | | |
| | IGHJ3*01) [8.8.8] (1-115)-IGHG1*03 | | |
| | R120 > K(116-445)], (218-216′)- | | |
| | disulfide with lambda light chain (1′-217′) | | |
| | [Homo sapiens V-LAMBDA | | |
| | (IGLV1-44*01 (88.20%)- | | |
| | IGLJ2*01G120 > T) [8.3.12] (1′-111′)- | | |
| | IGLC2*01 (112′-217′)]; (224-224″: | | |
| | 227-227″)-bisdisulfide dimer; | | |
| | Human IgG1 anti-IL-12/23 | | |
| | monoclonal antibody | | |
| Phase III | 2-(4-Ethoxyphenyl)-3-[4- | GW-406381X; | WO 1999012930; WO 2001045683; |
| | (methylsulfonyl)phenyl]pyrazolo[1,5- | 406381; | WO 2001056555; WO 2001056573; |
| | b]pyridazine | GW-406381 | WO 2003077920 |
| Phase III | Cyclo[L-alanyl-D-alanyl-N-methyl-L- | LX-211; R-1524; | US 2003139326; US 2003171264; |
| | leucyl-N-methyl-L-leucyl-N-methyl- | ISA-247; | US 2003212249; US 2009092665; |
| | L-valyl-[3(R)-hydroxy-N,4(R)- | trans-ISA-247; | U.S. Pat. No. 6,605,593; U.S. Pat. No. 6,613,739; |
| | dimethyl-L-2-amino-6(E),8- | ISAtx-247; | WO 1999018120; WO 2003053404; |
| | nonadienoyl]-L-(2-aminobutyryl)-N- | LX-214, | WO 2006014872; WO 2006066416; |
| | methylglycyl-N-methyl-L-leucyl-L- | Voclosporin, | WO 2009105692 |
| | valyl-N-methyl-L-leucyl]; | Luveniq | |
| | Cyclo[[3(R)-hydroxy-4(R)-methyl- | | |
| | 2(S)-(methylamino)-6(E),8- | | |
| | nonadienoyl]-L-(2-aminobutyryl)-N- | | |
| | methylglycyl-N-methyl-L-leucyl-L- | | |
| | valyl-N-methyl-L-leucyl-L-alanyl-D- | | |
| | alanyl-N-methyl-L-leucyl-N-methyl- | | |
| | L-leucyl-N-methyl-L-valyl]; 6-[3(R)- | | |
| | Hydroxy-4(R)-methyl-2(S)- | | |
| | (methylamino)-6(E),8-nonadienoic | | |
| | acid]cyclosporin A | | |
| Phase III | Immunoglobulin G1, anti-(human | HGS-1006; Anti- | WO 2009132058 |
| | cytokine BAFF) (human monoclonal | BLys, Belimumab, | |
| | LymphoStat-B heavy chain), disulfide | LymphoStat-B; | |
| | with human monoclonal LymphoStat- | Benlysta | |
| | B lamda-chain, dimer | | |
| Phase III | 3-[4(R)-Methyl-3(R)-[N-methyl-N- | CP-690550 citrate | US 2003130292; WO 2001042246; |
| | (7H-pyrrolo[2,3-d]pyrimidin-4- | | WO 2004047843; WO 2005051393; |
| | yl)amino]piperidin-1-yl]-3- | | WO 2005060972; WO 2005105146; |
| | oxopropanenitrile citrate | | WO 2006056399; WO 2006058007; |
| | | | WO 2007012953; WO 2007084630; |
| | | | WO 2007107318; WO 2008029237; |
| | | | WO 2009007839 |
| Phase III | 2(E)-Butenedioic acid dimethyl ester; | BG-12; FAG-201; | WO 1998052549; WO 1999049858; |
| | Fumaric acid dimethyl ester | BG-00012; | WO 2000012072; WO 2000023068; |
| | | AZL-O-211089; | WO 2000030622; WO 2001051047; |
| | | DMF, Dimethyl | WO 2002055067; WO 2005023241; |
| | | fumarate, Panaclar | WO 2005027899; WO 2006037342; |
| | | | WO 2007042034; WO 2007148744; |
| | | | WO 2009035251 |
| Phase III | Immunoglobulin G1, anti-(human | TRX-4; | WO 1993019196; WO 2007033230 |
| | CD3 (antigen)) (human-rat | ChAGCD3; | |
| | monoclonal heavy chain), disulfide | ChAglyCD3, | |
| | with human-rat monoclonal lamda- | Otelixizumab | |
| | chain, dimer; Unglycosylated | | |
| | immunoglobulin G1, anti-(human | | |
| | CD3 epsilon chain) humanized rat | | |
| | monoclonal YTH12.5; gamma 1 | | |
| | heavy chain [humanized VH (Homo | | |
| | sapiens FR/Rattus norvegicus CDR)- | | |
| | [297-alanine] Homo sapiens IGHG1] | | |
| | (222-214)-disulfide with chimeric | | |
| | lambda light chain [Rattus norvegicus | | |
| | VL/Homo sapiens IGLC2]; (228-228″: | | |
| | 231-231″)-bisdisulfide dimer; | | |
| | Immunoglobulin G1, anti-(human | | |
| | CD3E) humanized/chimeric | | |
| | monoclonal TRX4 (ChAglyCD3); | | |
| | humanized gamma1 heavy chain | | |
| | 299N > A [humanized VH (Homo | | |
| | sapiens FR/Rattus sp. CDR) (119 | | |
| | residues [8.8.12])-Homo sapiens | | |
| | IGHG1*01, 180N > A (CH2 84.4)] | | |
| | (222-216′)-disulfide with chimeric | | |
| | lambda light chain 111G > R [Rattus | | |
| | sp. V-LAMBDA (110 residues | | |
| | [8.3.9])-Homo sapiens IGLC2*01, | | |
| | 1G > R (1.5)]; (228-228′: 231-231′)- | | |
| | bisdisulfide dimer | | |
| Phase III | L-Glutaminyl-L-lysyl-L-arginyl-L- | dnaJP1; AT-001 | US 2002122818; WO 1995031984; |
| | alanyl-L-alanyl-L-tyrosyl-L-aspartyl- | | WO 2001080833; WO 2002012286; |
| | L-glutaminyl-L-tyrosyl-glycyl-L- | | WO 2002036611; WO 2003026579; |
| | histidyl-L-alanyl-L-alanyl-L- | | WO 2007143174 |
| | phenylalanyl-L-glutamic acid | | |
| Phase III | Immunoglobulin G1, anti-(human | PRO-70769; | WO 2004056312; WO 2005115453; |
| | CD20 [antigen]) (human-mouse | R-1594; h2H7; | WO 2005117972; WO 2005117978; |
| | monoclonal 2H7 gamma1-chain), | RG-1594, | WO 2005120437; WO 2006076651; |
| | disulfide with human-mouse | Ocrelizumab | WO 2007117600; WO 2008122007; |
| | monoclonal 2H7 kappachain, dimer | | WO 2009009523; WO 2009040268; |
| | | | WO 2009085765 |
| Phase III | Immunoglobulin G2-kappa, anti- | CP-751871, | WO 2002053596 |
| | [Homo sapiens insulin-like growth | Figitumumab | |
| | factor 1 receptor (IGF-1R, CD221)], | | |
| | Homo sapiens monoclonal antibody; | | |
| | gamma2 heavy chain (1-450) [Homo | | |
| | sapiens VH (IGHV3-23*01 (93.90%)- | | |
| | (IGHD)-IGHJ6*01) [8.8.18] (1-125)- | | |
| | IGHG2*01, CH3 K130 > del (126-450)], | | |
| | (139-214′)-disulfide with kappa | | |
| | light chain (1′-214′) [Homo sapiens V- | | |
| | KAPPA (IGKV1-17*01 (95.80%)- | | |
| | IGKJ2*04) [6.3.9] (1′-107′)- | | |
| | IGKC*01] (108′-214′); (227-227″:228-228″: | | |
| | 231-231″:234-234″)- | | |
| | tetradisulfide dimer; Immunoglobulin | | |
| | G2, anti-(human insulin-like growth | | |
| | factor 1 receptor (EC.2.7.10.1 or | | |
| | CD221 antigen)); human monoclonal | | |
| | CP-751,871 clone 2.13.2 gamma2 | | |
| | heavy chain (139-214′)-disulfide with | | |
| | human monoclonal CP-751,871 clone | | |
| | 2.13.2 kappa light chain, dimer (227-227″: | | |
| | 228-228″:231-231″:234-234″)- | | |
| | tetrakisdisulfide; Immunoglobulin G2, | | |
| | anti-(human insulin-like growth factor | | |
| | I receptor) (human monoclonal CP- | | |
| | 751,871 clone 2.13.2 heavy chain) | | |
| | disulfide with human monoclonal CP- | | |
| | 751,871 clone 2.13.2 light chain, | | |
| | dimer | | |
| Phase III | Combination of omeprazole and | PN-200, | |
| | naproxen | Omeprazole/ | |
| | | naproxen | |
| Phase III | Monoclonal antibody against human | Anti-IL-17 mAb; | WO 2006013107; WO 2007117749 |
| | IL-17 | NVP-AIN-457; | |
| | | AIN-457; KB- | |
| | | 03303A | |
| Phase III | 4-(4-Methylpiperazin-1-ylmethyl)-N- | AB-1010, | WO 2004096225; WO 2005016323; |
| | [4-methyl-3-[4-(3-pyridyl)thiazol-2- | Masitinib mesylate | WO 2007026251; WO 2008084103; |
| | ylamino]phenyl]benzamide | | WO 2008098949 |
| | methanesulfonate | | |
| Phase III | 3(S)-Cyclopentyl-3-[4-(7H- | INCB-18424; | US 2007135461; WO 2009073575 |
| | pyrrolo[2,3-d]pyrimidin-4-yl)-1H- | INCB-018424 | |
| | pyrazol-1-yl]propanenitrile | | |
| Pre-Registered | CTLA-4 (antigen) [29-tyrosine, 104- | LEA-029; | WO 2001092337; WO 2002002638; |
| | glutamic acid] (human extracellular | L104EA29YIg; | WO 2005016266; WO 2006107298; |
| | domain-containing fragment) fusion | BMS-224818; | WO 2006108035; WO 2007076354 |
| | protein with immunoglobulin G1 | LEA29Y, | |
| | (human monoclonal Fc domain- | Belatacept | |
| | containing fragment), bimol. (120--120′)- | | |
| | disulfide; | | |
| | [Tyr29,Glu104,Gln125,Ser130,Ser136, | | |
| | Ser139,Ser148](CTLA-4 (antigen)- | | |
| | [3-126]-peptide (human extracellular | | |
| | domain-containing fragment) fusion | | |
| | protein with immunoglobulin G1-[233 | | |
| | C-terminal residues of the heavy | | |
| | chain]-peptide (human monoclonal Fc | | |
| | domain-containing fragment)) | | |
| | bimolecular (120--120′)-disulfide | | |
| Pre-Registered | Fully human monoclonal antibody to | AMG-162, | WO 2007081879 |
| | receptor activator of NF-kappaB | Denosumab, Prolia | |
| | ligand (RANKL); Immunoglobulin | | |
| | G2, anti-(human osteoclast | | |
| | differentiation factor) (human | | |
| | monoclonal AMG162 heavy chain), | | |
| | disulfide with human monoclonal | | |
| | AMG162 light chain, dimer; | | |
| | Immunoglobulin G2, anti-(human | | |
| | receptor activator of NF-kappaB | | |
| | ligand) (human monoclonal AMG162 | | |
| | heavy chain), disulfide with human | | |
| | monoclonal AMG162 light chain, | | |
| | dimer | | |
| Pre-Registered | Combination of naproxen and | PN-400, | U.S. Pat. No. 6,365,184; WO 2002098352 |
| | esomeprazole magnesium | Naproxen/esomepr | |
| | | azole magnesium; | |
| | | Esomeprazole | |
| | | magnesium/ | |
| | | naproxen, Vimovo | |

Suitable regimens including doses and routes of administration for most of the active ingredients disclosed herein (with exception of a compound of formula (I) and a compound of formula (II), the dosing of which is disclosed herein) can be determined from readily-available reference sources relating to these drugs, for example Physicians' Desk Reference (PDR), 62nd edition, Montvale, N.J.: Thomson Healthcare (2008) and various Internet sources known to those of skill in the art.

### Examples of Pharmaceutical Compositions

Pharmaceutical compositions are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include a composition comprising one or more active ingredients and a pharmaceutically acceptable diluent(s), excipient(s), or carrier(s). In addition, one or more active ingredients are optionally administered as pharmaceutical compositions in which they are mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a composition comprising one or more active ingredients with other chemical components, such as carriers, stabilizers, diluents, dispersing agents, suspending agents, thickening agents, and/or excipients. The pharmaceutical composition facilitates administration of one or more modulatory agents to an organism. In practicing the methods of treatment or use provided herein, therapeutically effective amounts of one or more active ingredients are administered in a pharmaceutical composition to a mammal having a condition, disease, or disorder to be treated. Usually, the mammal is a human. A therapeutically effective amount varies depending on the severity and stage of the condition, the age and relative health of the subject, the potency of the one or more active ingredients used and other factors. Active ingredients are optionally used singly or in combination with one or more additional active ingredients as components of mixtures.

One or more active ingredients and combinations thereof may be administered by any suitable method. The pharmaceutical formulations described herein are optionally administered to a subject by single or multiple administration routes, including but not limited to, oral, enteral, parenteral (e.g., intravenous, intraarterial, intramuscular, intracardiac, intracranial, intraocular, intracereberal, subcutaneous, intraosseous infusion, intradermal, intrathecal, intratracheal, nasopharyngeal, intraperitoneal and intravesical infusion), intranasal, by inhalation, buccal, transmucosal, epidural, vaginal, intravitreal, topical, epicutaneous, rectal, transdermal or via a suitable implant device.

The pharmaceutical formulations described herein include, but are not limited to, aqueous solutions (e.g. when the one or more active ingredients are water soluble), aqueous liquid dispersions, self-emulsifying dispersions, solid solutions, liposomal dispersions, aerosols, solid dosage forms, powders, creams, immediate release formulations, controlled release formulations, fast melt formulations, tablets, capsules, pills, delayed release formulations, extended release formulations, pulsatile release formulations, multiparticulate formulations, and mixed immediate and controlled release formulations. The pharmaceutical formulations described herein should be stable under the conditions of manufacture and storage and should be preserved against the contaminating action of microorganisms (e.g. bacteria and fungi). The prevention of the action of microorganisms can be brought about by various antibacterial and antifungal agents, for example, parabens, chlorobutanol, phenol, sorbic acid, thimerosal and the like.

The pharmaceutical compositions will include a composition comprising one or more active ingredients in free acid or free-base form, or in a pharmaceutically acceptable salt form. In addition, the methods and pharmaceutical compositions described herein include the use of N-oxides, crystalline forms (also known as polymorphs), as well as active metabolites of active ingredients having the same type of activity. In some situations, active ingredients exist as tautomers. All tautomers are included within the scope of the agents presented herein. Additionally, in some embodiments, a composition comprising one or more active ingredients exists in unsolvated as well as solvated forms with pharmaceutically acceptable solvents such as water, ethanol, and the like. The solvated forms of the active ingredients presented herein are also considered to be disclosed herein.

“Carrier materials” include any commonly used excipients in pharmaceutics and should be selected on the basis of compatibility with the active ingredients disclosed herein, such as a composition comprising tranilast and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include one or more active ingredients, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use are optionally obtained by mixing one or more solid excipients with a composition comprising one or more active ingredients, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of one or more active ingredients are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations comprising one or more active ingredients, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

The solid oral pharmaceutical dosage forms including formulations described herein, are optionally further formulated to provide a controlled release of the one or more active ingredients. Controlled release refers to the release of a composition comprising one or more active ingredients from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an active ingredient to a subject over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which can comprise one or more active ingredients can be delivered using a pulsatile dosage form. A pulsatile dosage form is capable of providing one or more immediate release pulses at predetermined time points after a controlled lag time or at specific sites. Pulsatile dosage forms including the formulations described herein, which comprise one or more active ingredients, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to compositions comprising one or more active ingredients, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, compositions comprising one or more active ingredients (e.g. tranilast) are optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of compositions comprising one or more active ingredients and a suitable powder base such as lactose or starch.

Buccal formulations that comprise one or more active ingredients include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the modulatory agent, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of one or more active ingredients can be administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of one or more active ingredients disclosed herein (e.g. tranilast) employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of one or more active ingredients are optionally accomplished by means of iontophoretic patches and the like. Additionally, transdermal patches provide controlled delivery of one or more active ingredients. The rate of absorption is optionally slowed by using rate-controlling membranes or by trapping active ingredients within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing one or more active ingredients optionally with carriers, optionally a rate controlling barrier to deliver one or more active ingredients to the skin of the host at a controlled and predetermined rate over a prolonged period of time, and means to secure the device to the skin.

Formulations that comprise one or more active ingredients suitable for injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (e.g. propylene glycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, one or more active ingredients are optionally formulated in aqueous solutions, generally in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, generally with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of one or more active ingredients in water soluble form. Additionally, suspensions of one or more active ingredients are optionally prepared as appropriate oily injection suspensions.

In some embodiments, one or more active ingredients are administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

One or more active ingredients are also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

One or more active ingredients are optionally used in the preparation of medicaments for the prophylactic and/or therapeutic treatment of inflammatory conditions or conditions that would benefit, at least in part, from amelioration. In addition, a method for treating any of the diseases or conditions described herein involves administration to a subject in need of such treatment, a pharmaceutical composition containing one or more active ingredients as described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In the case wherein the patient's condition does not improve, upon the doctor's discretion the administration of one or more active ingredients are optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of one or more active ingredients are optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 clays, 300 days, 320 days, 350 days, or 365 days. The dote reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical composition described herein is in unit dosage forms suitable for single administration of precise dosages. In unit dosage form, the formulation is divided into unit doses containing appropriate quantities of the one or more active ingredients. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The active ingredients and combinations thereof disclosed herein are contemplated to exhibit therapeutic activity when administered in an amount which can depend on the particular case. The variation in amount can depend, for example, on the human or animal and the active ingredients chosen. A broad range of doses can be applicable. Considering a subject, for example, from about 0.01 mg to about 500 mg of a modulatory agent or active ingredient may be administered per kilogram of body weight per day. For example, a compound of formula (A), a compound of formula (I), a compound of formula (II), tranilast or a pharmaceutically acceptable salts thereof, can be administered from about 0.001 mg, 0.01 mg, 0.1 mg, 0.5 mg, 1 mg, 5 mg, 10 mg, 50 mg, 100 mg, 250 mg to about 500 mg of tranilast per kilogram of body weight per day. In various embodiments a pharmaceutical composition of the invention is configured to provide a daily dosage of one or more active ingredients from between about 0.001 mg, 0.01 mg, 0.1 mg, 0.5 mg, 1 mg, 5 mg, 10 mg, 50 mg, 100 mg, 200 mg, 250 mg, 300 mg, 350 mg, 400 mg, 500 mg, 600 mg, 700 mg, 800 mg, 900 mg, 1000 mg, 1500 mg, to about 2000 mg per kg of body weight. An indicated daily dosage in a larger mammal, including, but not limited to, humans, can be in the range from about 0.5 mg to about 5000 mg, conveniently administered in divided doses, including, but not limited to, up to four times a day or in extended release form. Dosage regimes may be adjusted to provide the optimum therapeutic response. For example, several divided doses may be administered daily, weekly, monthly or other at suitable time intervals or the dose may be proportionally reduced as indicated by the exigencies of the situation. Suitable unit dosage forms for oral administration can include from about 1 to 5000 mg active ingredient. Generally, a compound of formula (A), of formula (I), of formula (II), tranilast or a pharmaceutically acceptable salt thereof is administered at between about 50 and about 1000 mg per day or between about 100 to about 900 mg per day. Usually, a compound of formula (A), of formula (I), of formula (II), tranilast or a pharmaceutically acceptable salt thereof is administered at between about 150 and 600 mg per day or between about 300 and about 600 mg per day. Such doses include 150, 175, 200, 225, 250, 275, 300, 325, 350, 375, 400, 425, 450, 475, 500, 525, 550, 575, or 600 mg per day. The foregoing ranges are merely suggestive, as the number of variables in regard to an individual treatment regime is large, and considerable excursions from these recommended values are not uncommon. Such dosages are optionally altered depending on a number of variables, not limited to the activity of the one or more active ingredients used, the disease or condition to be treated, the mode of administration, the requirements of the individual subject, the severity of the disease or condition being treated, and the judgment of the practitioner. The daily dosages appropriate for one or more active ingredients, other than compounds of formula (A), of formula (I), of formula (II) and tranilast can be any dose that is suitable for the condition to be treated.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. Active ingredients exhibiting high therapeutic indices is preferred. The data obtained from cell culture assays and animal studies are optionally used in formulating a range of dosage for use in human. The dosage of such active ingredients generally lies within a range of circulating concentrations that include the ED50 with minimal toxicity. The dosage optionally varies within this range depending upon the dosage form employed and the route of administration utilized.

### Combination Treatments

The agents described herein, where combinational therapy is employed, do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

Therapeutically effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, multiple therapeutic agents can be administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents. The use of multiple therapeutic combinations are also envisioned.

It is understood that the dosage regimen to treat, prevent, or ameliorate the condition(s) for which relief is sought, is optionally modified in accordance with a variety of factors. These factors include the condition from which the subject suffers, as well as the age, weight, sex, diet, and medical condition of the subject. Thus, the dosage regimen actually employed varies widely, in some embodiments, and therefore deviates from the dosage regimens set forth herein. The pharmaceutical agents which make up the combination therapy disclosed herein are optionally a combined dosage form (e.g. combined in the same formulation) or in separate dosage forms (e.g. two or more different formulations) intended for substantially simultaneous administration. Simultaneous administration can be by the same route or by different routes. The pharmaceutical agents that make up the combination therapy can optionally be administered sequentially, with either therapeutic agent being administered by a regimen calling for multi-step administration. The multi-step administration regimen optionally calls for sequential administration of the active agents or spaced-apart administration of the separate active agents. By “sequential” administration is meant a time difference of from seconds, minutes, hours or days between the two or more administration steps of the two or more active ingredients. The two or more agents may be administered in any order. The time period between the multiple administration steps may depend upon the properties of each pharmaceutical agent, such as potency, solubility, bioavailability, plasma half-life and kinetic profile of the pharmaceutical agent. Circadian variation of the target molecule concentrations are optionally used to determine the optimal dose interval. Dosing can also be influenced by the fed or fasted state of the patient. For example, one or more therapeutic agents can be administered with meals, or on an empty stomach, such as at least one hour before eating.

In addition, a modulatory agent (e.g. tranilast) is optionally used in combination with procedures that provide additional or synergistic benefit to the patient. By way of example only, patients are expected to find therapeutic and/or prophylactic benefit in the methods described herein, wherein pharmaceutical compositions of a modulatory agent with other therapeutics are combined with genetic testing to determine whether that individual is a carrier of a mutant gene that is correlated with a certain disease or condition.

Compositions comprising two or more active ingredients (e.g. tranilast and at least one other active ingredient) can be administered before, during or after the occurrence of a disease or condition, and the timing of administering the composition varies in some embodiments. Thus, for example, a composition comprising tranilast and at least one other active ingredient can be used as a prophylactic and can be administered continuously to subjects at risk of developing a condition or disease (e.g. rheumatoid arthritis) in order to prevent the occurrence of the disease or condition. Said subjects may be asymptomatic. For example a subject may be positive for one or more autoantibodies that indicate the subject is at risk of developing rheumatoid arthritis. Compositions comprising two or more active ingredients can be administered to a subject during or as soon as possible after the onset of the symptoms. For example compositions comprising two or more active ingredients can be administered within the first 48 hours of the onset of the symptoms. In some embodiments the compositions can be administered within the first 6 hours of the onset of the symptoms or within 3 hours after the onset of the symptoms. The initial administration can be via any suitable route. Compositions comprising two or more active ingredients as disclosed herein can be administered as soon as is practicable after the onset of a disease or condition is detected or suspected, and for any length of time necessary for the treatment of the disease.

Tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain one or more active ingredients, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the one or more active ingredients may be incorporated into sustained-release preparations and formulations as described herein.

While the present invention has been described in conjunction with the specific embodiments set forth above, many alternatives, modifications and variations thereof will be apparent to those of ordinary skill in the art. Thus, for example, classes of known anti-arthritic agents not recited above are within the scope of the invention, as are known but unrecited species of recited classes of anti-arthritic agents. Similarly, the treatment of known but unrecited arthritic conditions is within the scope of the invention. All such alternatives, modifications, and variations are intended to fall within the spirit and scope of the present invention.

### Kits

Kits are contemplated for use herein. In one embodiment, a kit comprises a first dosage form comprising tranilast in one or more of the forms identified above (e.g. a tablet, capsule, pill, delayed release formulation) and at least a second dosage form comprising one or more of the forms identified above, in quantities sufficient to carry out the methods of the present invention. The second dosage form, and any additional dosage forms (e.g. a third, fourth of fifth dosage form) can comprise any active ingredient disclosed herein for the treatment of an arthritic condition. All dosage forms together can comprise a therapeutically effective amount of each compound for the treatment of an arthritic condition. In some embodiments a kit is for a subject with an arthritic condition to use in the self-administration of at least one oral agent, wherein the kit comprises a container housing a plurality of said oral agents and instructions for carrying out drug administration therewith. The at least one oral agent can comprise a combination of a therapeutically effective dose of tranilast and a therapeutically effective dose of an agent selected from the group consisting of a DMD (e.g. a DMAOD or a TNF antagonist), an NSAID, a Cox-2 inhibitor, an antibiotic and an analgesic. In some embodiments a kit for use by a subject with an arthritic condition comprises at least one oral agent, a container housing a plurality of said oral agents and instructions for carrying out drug administration therewith, wherein said at least one oral agent comprises a combination of a therapeutically effective daily dose of tranilast, or a pharmaceutically acceptable salt thereof and a daily dose of an agent selected from the group consisting of a DMD (e.g. a DMAOD or a TNF antagonist), an NSAID, a Cox-2 inhibitor, an antibiotic and an analgesic. In some embodiments the agent can be in distinct individual dosage forms or combined in a single dosage form or a combination of dosage forms thereof. In some embodiments tranilast, or a pharmaceutically acceptable salt thereof is in a distinct individual dosage form or combined in a single dosage form with the agent or a combination of dosage forms thereof.

### EXAMPLES

The benefit of a combination therapy that includes tranilast and an additional therapy for the treatment of an arthritic conditions is demonstrated in the following examples.

Oral dosage formulations of tranilast can be generated by any suitable method including, but not limited to those methods previously disclosed herein. In addition, tranilast, for use in tablets, and for use in the examples that follow can be prepared as described in U.S. Ser. No. 09/902,822 or PCT/US 01/21860. Methods for preparing such dosage forms are known.

### Deuterated Analogs

There are a number of synthetic pathways which yield a deuterated analog of tranilast. Scheme 1 describes but one method to prepare such a deuterated analog; other methods are well-known to those of skill in the art. Following a standard amide synthesis, such as that shown below, the starting material, deuterated anthranilic acid, A-1 (CAS 60124-83-6), can be reacted with a cinnamic acid analog, B-1, to yield a deuterated analog of tranilast, C-1.

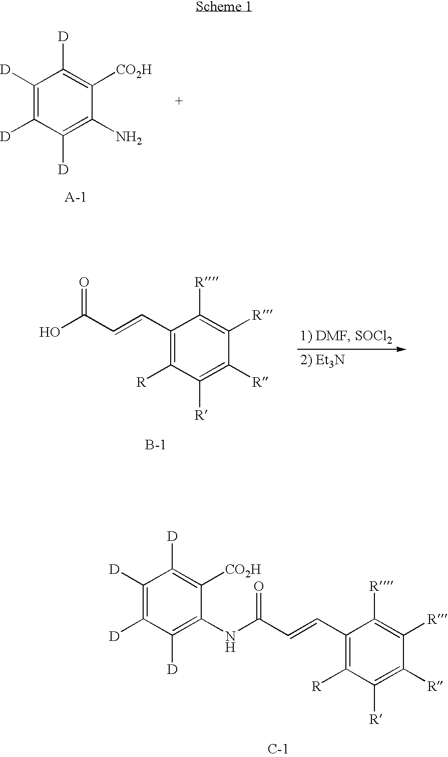

Alternately, a deuterated cinnamic acid analog, B-2, can be prepared from a deuterated dimethoxybenzaldehyde derivative, such as:

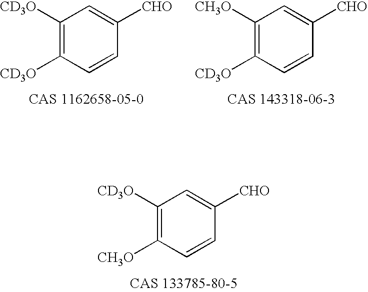

([CAS 1162658-05-0] See Zou et al. Chemistry Express 1991 6(3):213-216 “Synthesis of 1,2,4-trimethoxy benzene and its three monomethoxy-d₃ derivatives;” [CAS 143318-06-3 and CAS 1337-80-5] see US 2009/0062300 to Czarnik)

A deuterated dimethoxybenzaldehyde derivative can be converted to a deuterated cinnamic acid analog according to Scheme 2, where each of R^{a} and R^{b} is independently —CH₃ or —CD₃.

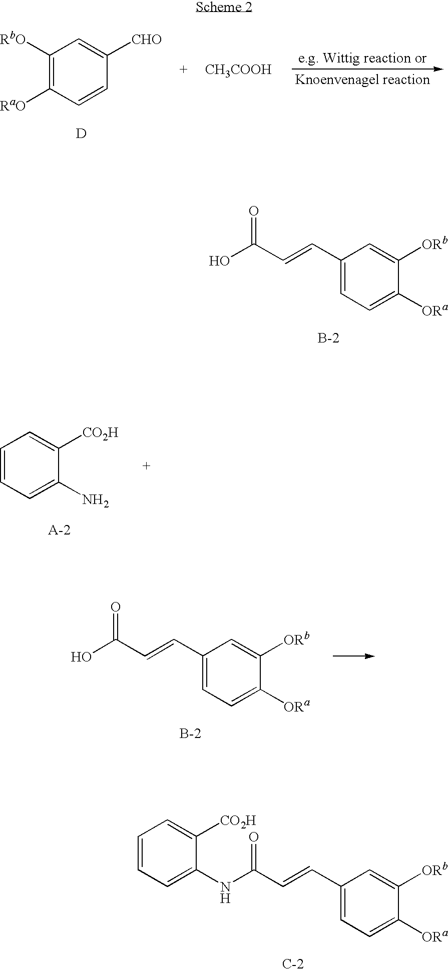

### Example A

### Synthesis of Deuterated Tranilast (C-1)

To a solution of a cinnamic acid analog (B-1) (1 equiv.) in anhydrous DCM and catalytic DMF, thionyl chloride (1.1 equiv.) is added at 0-5° C. The reaction is refluxed for 1 h and evaporated under reduced pressure. The residue is triturated with DCM and evaporated. The acid chloride is then dissolved in DCM and added to a solution of deuterated anthranilic acid (A-1, C/D/N Isotopes (Pointe-Claire, Quebec Canada)) (0.9 equiv.) and triethylamine (2-4 equiv.) in DCM at 0-5° C. The reaction is monitored by TLC and product is isolated after washing the reaction mixture with saturated aq. NaCl solution (X3), is dried over anhydrous Na₂SO₄ and is evaporated. The crude product (C-1) is purified by column chromatography.

### Example B

### Synthesis of Deuterated Tranilast (C-2)

### Example B-1

### Synthesis of Deuterated Cinnamic Acid Analog (B-2)

Deuterated cinnamic acid analog (B-2) is prepared by the Doebner modification of the Knoevenagel condensation of deuterated dimethoxybenzaldehyde derivative (D) and malonic acid in pyridine. The reaction is carried out as described for the synthesis of 2,3-dimethoxycinnamic acid in Organic Synthesis, Collected Vol. 4, pp 327-329. D (0.01 mol) and malonic acid (0.02 mol) in pyridine (10 mL) are heated and when the malonic acid is dissolved, piperidine (0.2 mL) is added. The reaction is heated as described in the above reference and worked up using conditions as described to afford B-2.

### Example B-2

### Synthesis of Deuterated Tranilast C-2

To a solution of a cinnamic acid analog (B-2) (1 equiv.) in anhydrous DCM and catalytic DMF, thionyl chloride (1.1 equiv.) is added at 0-5° C. The reaction is refluxed for 1 h and evaporated under reduced pressure. The residue is triturated with DCM and evaporated. The acid chloride is then dissolved in DCM and added to a solution of anthranilic acid (A-2) (0.9 equiv.) and triethylamine (2-4 equiv.) in DCM at 0-5° C. The reaction is monitored by TLC and product is isolated after washing the reaction mixture with saturated aq. NaCl solution (X3), is dried over anhydrous Na₂SO₄ and is evaporated. The crude product (C-2) is purified by column chromatography.

### Treatment of Rheumatoid Arthritis

### Example 1

### Clinical Treatment of Rheumatoid Arthritis with Tranilast and a TNF Antagonist

A randomized, double-blind, placebo controlled study is conducted to evaluate the safety and efficacy of tranilast, alone or in combination with a TNF antagonist (e.g. etanercept, adalimumab or infliximab). Tranilast is formulated and prepared for oral administration. Tranilast is administered by multiple doses of 2, 20 or 200 mg/kg tranilast, twice daily alone or in combination with a TNF antagonist, in the treatment of rheumatoid arthritis (RA) in human patients. Etanercept is administered by subcutaneous (s.c.) injection, twice weekly at a dose of 25 mg per administration. Adalimumab is administered at 40 mg, s.c., twice a week for 2 weeks, then weekly for the next 10 weeks and then every other week. Infliximab is administered at 3 mg/kg given as an intravenous infusion at weeks 1, 2 and 6 and then every 8 weeks thereafter. Tranilast or placebo is administered alone or in combination with either etanercept, adalimumab, infliximab or a suitable saline placebo. Methotrexate is administered orally at 7.5 milligrams, once a week.

Five hundred (500) patients at multiple European centers with a prior history of treatment for at least 6 months with methotrexate and optionally, NSAIDs and with active disease (according to the criteria of the American College of Rheumatology) with erosive changes on X-rays of hands and feet, are enrolled in the trial. Active disease is defined by the presence of six or more swollen joints plus at least three of four secondary criteria (duration of morning stiffness>=45 minutes; >=45 tender or painful joints; erythrocyte sedimentation rate (ESR)>=28 mm/hour; C-reactive protein (CRP)>=220 mg/l.

In patients using NSAIDs, the doses are to remain stable for 4 weeks prior to screening and also throughout trial participation. Patients are randomized to one of seventeen treatment groups (Group 1, tranilast (2 mg/kg) and placebo; Group 2, tranilast (2 mg/kg) and etanercept; Group 3, tranilast (2 mg/kg) and adalimumab; Group 4, tranilast (2 mg/kg) and infliximab; Group 5, placebo and etanercept; Group 6, placebo and adalimumab; Group 7, placebo and infliximab; Group 8, placebo, Group 9, placebo and methotrexate; Group 10, tranilast (20 mg/kg) and placebo; Group 11, tranilast (20 mg/kg) and etanercept; Group 12, tranilast (20 mg/kg) and adalimumab; Group 13, tranilast (20 mg/kg) and infliximab; Group 14, tranilast (200 mg/kg) and placebo; Group 15, tranilast (200 mg/kg) and etanercept; Group 16, tranilast (200 mg/kg) and adalimumab; Group 17, tranilast (200 mg/kg) and infliximab). Patients are monitored for adverse events during treatment and regularly thereafter, by interviews, physical examination, and laboratory testing.

Six primary disease-activity assessments are chosen to allow analysis of the response in individual patients according to the Paulus index (Paulus, et al., Arthritis Rheumatism 33:477-484 (1990), the teachings of which are incorporated herein by reference). The assessments contributing to this index are tender joint and swollen joint scores (60 and 58 joints, respectively, hips not assessed for swelling; graded 0-3), the duration of morning stiffness (minutes), the patient's and physician's assessment of disease severity (on a 5-point scale, ranging from 1 (symptom-free) to 5 (very severe), and erythrocyte sedimentation rate (ESR). Patients are considered to have responded if at least four of the six variables improved, defined as at least 20% improvement in the continuous variables, and at least two grades of improvement or improvement from grade 2 to 1 in the two disease-severity assessments (Paulus 20% response). Improvements of at least 50% in the continuous variables are also used (Paulus 50% response).

Other disease-activity assessments include a pain score (0-10 cm on a visual analogue scale (VAS)), an assessment of fatigue (0-10 cm VAS), and grip strength (0-300 mm Hg, mean of three measurements per hand by sphygmomanometer cuff).

An erythrocyte sedimentation rate (ESR) is measured at each study site with a standard method (Westergen). C-reactive protein (CRP) is measured by rate nephelometry (Abbott fluorescent polarizing immunoassay). See also, Elliott et al., Lancet 344:1105-1110 (1994); Elliott et al., Lancet 344:1125-1127 (1994); and Elliott et al., Arthritis Rheum. 36(12):1681-1690 (1993), which references are entirely incorporated herein by reference.

Evaluations are performed at weeks 1, 2, 4, 6, 8, 10, 12, 14, 16, 18, 20, 22 and 26. The patients enrolled in each group are well matched for baseline demographics. Disease duration and swollen and tender joint counts at baseline are also well-balanced across the groups. Results are tabulated and presented in a table format and are analyzed by a skilled artesian to determine a recommended dosage regime for a subject with rheumatoid arthritis.

### Example 2

### Clinical Treatment of Rheumatoid Arthritis with Tranilast and a Glucocorticoid

A randomized, double-blind, placebo controlled study is conducted to evaluate the safety and efficacy of tranilast, alone or in combination with a glucocorticoid (e.g. budesonide, prednisone, prednisolone, or methylprednisolone). Tranilast is formulated and prepared for oral administration. Tranilast is administered by multiple doses of 2, 20 or 200 mg/kg tranilast, twice daily alone or in combination with a glucocorticoid, in the treatment of rheumatoid arthritis (RA) in human patients. Budesonide, prednisone, prednisolone, or methylprednisolone are administered orally at 20 mg per day. Methotrexate is administered orally at 7.5 milligrams, once a week.

Five hundred (500) patients at multiple European centers with a prior history of treatment for at least 6 months with methotrexate and optionally, NSAIDs and with active disease (according to the criteria of the American College of Rheumatology) with erosive changes on X-rays of hands and feet, are enrolled in the trial. Active disease is defined by the presence of six or more swollen joints plus at least three of four secondary criteria (duration of morning stiffness>=45 minutes; >=45 tender or painful joints; erythrocyte sedimentation rate (ESR)>=28 mm/hour; C-reactive protein (CRP)>=220 mg/l.

In patients using NSAIDs, the doses are to remain stable for 4 weeks prior to screening and also throughout trial participation. Patients are randomized to one of seventeen treatment groups (Group 1, tranilast (2 mg/kg) and placebo; Group 2, tranilast (2 mg/kg) and budesonide; Group 3, tranilast (2 mg/kg) and prednisone; Group 4, tranilast (2 mg/kg) and methylprednisolone; Group 5, placebo and budesonide; Group 6, placebo and prednisone; Group 7, placebo and methylprednisolone; Group 8, placebo, Group 9, placebo and methotrexate; Group 10, tranilast (20 mg/kg) and placebo; Group 11, tranilast (20 mg/kg) and budesonide; Group 12, tranilast (20 mg/kg) and prednisone; Group 13, tranilast (20 mg/kg) and methylprednisolone; Group 14, tranilast (200 mg/kg) and placebo; Group 15, tranilast (200 mg/kg) and budesonide; Group 16, tranilast (200 mg/kg) and prednisone; Group 17, tranilast (200 mg/kg) and methylprednisolone). Patients are monitored for adverse events during treatment and regularly thereafter, by interviews, physical examination, and laboratory testing.

Disease-activity assessments, including laboratory testing are conducted as disclosed in Example 1.

Evaluations are performed at weeks 1, 2, 4, 6, 8, 10, 12, 14, 16, 18, 20, 22 and 26. The patients enrolled in each group are well matched for baseline demographics. Disease duration and swollen and tender joint counts at baseline are also well-balanced across the groups. Results are tabulated and presented in a table format and are analyzed by a skilled artesian to determine a recommended dosage regime for a subject with rheumatoid arthritis.

### Example 3

### Clinical Treatment of Rheumatoid Arthritis with Tranilast and an NSAID

NSAIDs that are contemplated for use herein to treat arthritic conditions include salicylic acid derivatives (such as salicylic acid, acetylsalicylic acid, methyl salicylate, diflunisal, olsalazine, salsalate and sulfasalazine), indole and indene acetic acids (such as indomethacin, etodolac and sulindac), fenamates (such as etofenamic, meclofenamic, mefenamic, flufenamic, niflumic and tolfenamic acids), heteroaryl acetic acids (such as acemetacin, alclofenac, clidanac, diclofenac, fenchlofenac, fentiazac, furofenac, ibufenac, isoxepac, ketorolac, oxipinac, tiopinac, tolmetin, zidometacin and zomepirac), aryl acetic acid and propionic acid derivatives (such as alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen), enolic acids (such as the oxicam derivatives ampiroxicam, cinnoxicam, droxicam, lornoxicam, meloxicam, piroxicam, sudoxicam and tenoxicam, and the pyrazolone derivatives aminopyrine, antipyrine, apazone, dipyrone, oxyphenbutazone and phenylbutazone), alkanones (such as nabumetone), nimesulide, proquazone, MX-1094, licofelone.

A randomized, double-blind, placebo controlled study is conducted to evaluate the safety and efficacy of tranilast, alone or in combination with an NSAID (e.g. ibuprofen, aspirin, and naproxen). Tranilast is formulated and prepared for oral administration. Tranilast is administered by multiple doses of 2, 20 or 200 mg/kg tranilast, twice daily alone or in combination with a glucocorticoid, in the treatment of rheumatoid arthritis (RA) in human patients. Ibuprofen (800 mg) and aspirin (600 mg) are administered orally, 4 times daily, and naproxen is administered orally at 500 mg twice daily. Methotrexate is administered orally at 7.5 milligrams, once a week.

Five hundred (500) patients at multiple European centers with a prior history of treatment for at least 6 months with methotrexate and with active disease (according to the criteria of the American College of Rheumatology) with erosive changes on X-rays of hands and feet, are enrolled in the trial. Active disease is defined by the presence of six or more swollen joints plus at least three of four secondary criteria (duration of morning stiffness>=45 minutes; >=45 tender or painful joints; erythrocyte sedimentation rate (ESR)>=28 mm/hour; C-reactive protein (CRP)>=220 mg/1.

Patients are randomized to one of seventeen treatment groups (Group 1, tranilast (2 mg/kg) and placebo; Group 2, tranilast (2 mg/kg) and ibuprofen; Group 3, tranilast (2 mg/kg) and aspirin; Group 4, tranilast (2 mg/kg) and naproxen; Group 5, placebo and ibuprofen; Group 6, placebo and aspirin; Group 7, placebo and naproxen; Group 8, placebo, Group 9, placebo and methotrexate; Group. 10, tranilast (20 mg/kg) and placebo; Group 11, tranilast (20 mg/kg) and ibuprofen; Group 12, tranilast (20 mg/kg) and aspirin; Group 13, tranilast (20 mg/kg) and naproxen; Group 14, tranilast (200 mg/kg) and placebo; Group 15, tranilast (200 mg/kg) and ibuprofen; Group 16, tranilast (200 mg/kg) and aspirin; Group 17, tranilast (200 mg/kg) and naproxen). Patients are monitored for adverse events during treatment and regularly thereafter, by interviews, physical examination, and laboratory testing.

Disease-activity assessments, including laboratory testing are conducted as disclosed in Example 1.

Evaluations are performed at weeks 1, 2, 4, 6, 8, 10, 12, 14, 16, 18, 20, 22 and 26. The patients enrolled in each group are well matched for baseline demographics. Disease duration and swollen and tender joint counts at baseline are also well-balanced across the groups. Results are tabulated and presented in a table format and are analyzed by a skilled artesian to determine a recommended dosage regime for a subject with rheumatoid arthritis.

### Example 4

### Clinical Treatment of Osteoarthritis with Tranilast and a DMOAD

DMOADs that are contemplated for use herein include glucosamine, chondroitin sulfate, calcitonin, alendronate, risedronate, zoledronic acid, teriparatide, VX-765, pralnacasan, SB-462795, CPA-926, ONO-4817, S-3536, PG-530742, CP-544439, pharmaceutically acceptable salts thereof, and combinations thereof.

A randomized, double-blind, placebo controlled study is conducted to evaluate the safety and efficacy of tranilast, alone or in combination with a DMOAD (e.g. glucosamine, chondroitin sulfate, and calcitonin). Tranilast is formulated and prepared for oral administration. Tranilast is administered by multiple doses of 2, 20 or 200 mg/kg tranilast, twice daily alone or in combination with a DMOAD, in the treatment of osteoarthritis in human patients. Glucosamine (1200 mg/d) and chondroitin sulfate (1500 mg/d) are administered orally and calcitonin (5.0 mg) is administered orally twice daily. Methotrexate is administered orally at 7.5 milligrams, once a week.

Five hundred (500) patients at multiple European centers with a prior history of treatment for at least 6 months with methotrexate and optionally, NSAIDs and with active disease (according to the criteria of the American College of Rheumatology) with erosive changes on X-rays of hands and feet, are enrolled in the trial. Active disease is defined by the presence of six or more swollen joints plus at least three of four secondary criteria (duration of morning stiffness>=45 minutes; >=45 tender or painful joints; erythrocyte sedimentation rate (ESR)>=28 mm/hour; C-reactive protein (CRP)>=220 mg/l.

In patients using NSAIDs, the doses are to, remain stable for 4 weeks prior to screening and also throughout trial participation. Patients are randomized to one of seventeen treatment groups (Group 1, tranilast (2 mg/kg) and placebo; Group 2, tranilast (2 mg/kg) and glucosamine; Group 3, tranilast (2 mg/kg) and chondroitin sulfate; Group 4, tranilast (2 mg/kg) and calcitonin; Group 5, placebo and glucosamine; Group 6, placebo and chondroitin sulfate; Group 7, placebo and calcitonin; Group 8, placebo, Group 9, placebo and methotrexate; Group 10, tranilast (20 mg/kg) and placebo; Group 11, tranilast (20 mg/kg) and glucosamine; Group 12, tranilast (20 mg/kg) and chondroitin sulfate; Group 13, tranilast (20 mg/kg) and calcitonin; Group 14, tranilast (200 mg/kg) and placebo; Group 15, tranilast (200 mg/kg) and glucosamine; Group 16, tranilast (200 mg/kg) and chondroitin sulfate; Group 17, tranilast (200 mg/kg) and calcitonin). Patients are monitored for adverse events during treatment and regularly thereafter, by interviews, physical examination, and laboratory testing.

Disease-activity assessments, including laboratory testing are conducted as disclosed in Example 1.

Evaluations are performed at weeks 1, 2, 4, 6, 8, 10, 12, 14, 16, 18, 20, 22 and 26. The patients enrolled in each group are well matched for baseline demographics. Disease duration and swollen and tender joint counts at baseline are also well-balanced across the groups. Results are tabulated and presented in a table format and are analyzed by a skilled artesian to determine a recommended dosage regime for a subject with rheumatoid arthritis.

### Example 5

### Clinical Treatment of Rheumatoid Arthritis with Tranilast, Methotrexate and/or a Cox-2 Inhibitor

COX-2 inhibitors that are contemplated for use herein to treat arthritic conditions include celecoxib, deracoxib, valdecoxib, parecoxib, rofecoxib, etoricoxib, lumiracoxib, PAC-10549, cimicoxib, GW-406381, LAS-34475, CS-502, pharmaceutically acceptable salts thereof, and combinations thereof.

A randomized, double-blind, placebo controlled study is conducted to evaluate the safety and efficacy of tranilast, alone or in combination with a COX-2 inhibitor and/or methotrexate. Tranilast is formulated and prepared for oral administration. Tranilast is administered by multiple doses of 2, 20 or 200 mg/kg tranilast, twice daily alone or in combination with celecoxib and/or methotrexate, in the treatment of rheumatoid arthritis (RA) in human patients. Celecoxib (100 mg) is administered orally twice daily. Methotrexate (MTX) is administered orally at 7.5 milligrams, once a week.

Four hundred (400) patients at multiple European centers with a prior history of disease for at least 6 months and with active disease (according to the criteria of the American College of Rheumatology) with erosive changes on X-rays of hands and feet, are enrolled in the trial. Active disease is defined by the presence of six or more swollen joints plus at least three of four secondary criteria (duration of morning stiffness>=45 minutes; >=45 tender or painful joints; erythrocyte sedimentation rate (ESR)>=28 mm/hour; C-reactive protein (CRP)>=220 mg/l.

Patients are randomized to one of twelve treatment groups (Group 1, tranilast (2 mg/kg) and placebo; Group 2, tranilast (20 mg/kg) and placebo; Group 3, tranilast (200 mg/kg) and placebo; Group 4, tranilast (2 mg/kg) and celecoxib; Group 5, tranilast (20 mg/kg) and celecoxib; Group 6, tranilast (200 mg/kg) and celecoxib; Group 7, tranilast (2 mg/kg) and MTX; Group 8, tranilast (20 mg/kg) and MTX; Group 9, tranilast (200 mg/kg) and MTX; Group 10, placebo and MTX; Group 11, placebo and celecoxib; and Group 12, placebo. Patients are monitored for adverse events during treatment and regularly thereafter, by interviews, physical examination, and laboratory testing.

Disease-activity assessments, including laboratory testing are conducted as disclosed in Example 1.

Evaluations were performed at weeks 1, 2, 4, 6, 8, 10, 12, 14, 16, 18, 20, 22 and 26. The patients enrolled in each group are well matched for baseline demographics. Disease duration and swollen and tender joint counts at baseline are also well-balanced across the groups. Results are tabulated and presented in a table format and are analyzed by a skilled artesian to determine a recommended dosage regime for a subject with rheumatoid arthritis.

### Example 6

### A Phase II, Randomized Multi-Center, Double-Blind Study of Tranilast with Concomitant Methotrexate (MTX) Compared to MTX Alone in Patients with Active Rheumatoid Arthritis (RA)

The purpose of this study is to evaluate whether tranilast at two different dosages compared to placebo is effective in patients with active RA when added to continuing methotrexate (MTX) therapy.

Primary Outcome Measures: Proportion of subjects who achieve ACR20 response with 12 week evaluation.

The clinical response studies were based on the criteria established by the American College of Rheumatology (ACR). A subject satisfied the ACR20 criterion if there was a 20 percent improvement in tender and swollen joint counts and 20 percent improvement in three of the five remaining symptoms measured, such as patient and physician global disease changes, pain, disability, and an acute phase reactant (Felson, D. T., et al., 1993 Arthritis and Rheumatism 36:729-740; Felson, D. T., et al., 1995 Arthritis and Rheumatism 38:1-9). Similarly, a subject satisfied the ACR50 or ACR70 criterion if there was a 50 or 70 percent improvement, respectively, in tender and swollen joint counts and 50 or 70 percent improvement, respectively, in three of the five remaining symptoms measured, such as patient and physician global disease changes, pain, physical disability, and an acute phase reactant such as CRP or ESR.

Secondary Outcome Measures: Proportion of subjects achieving ACR50 and ACR70 response; EULAR responders (e.g. Disease Activity Score using 28 joint counts (DAS28) good or moderate responders); mean change from baseline of each ACR component at weeks 2, 4, 8, 12 and 16; the safety and tolerability of both doses of tranilast.

Dosing: (a) Tranilast 150 mg tranilast tablets, bid, for 12 weeks; (b) Tranilast 75 mg tablets, bid for 12 weeks; Placebo Placebo tablets, bid for 12 weeks.

Inclusion Criteria: Two hundred fifty (250) rheumatoid arthritis patients with a diagnosis based upon the 1987 Revised American Rheumatism Association Criteria for the Classification of Rheumatoid Arthritis; Functional Class 1 3 defined by the 1991 Revised Criteria for the Classification of Global Functional Status in Rheumatoid Arthritis and active disease: >8 tender and >6 swollen joint counts (based upon 68/66 joint counts) and an elevated CRP level (defined as >the upper limit of normal [ULN] for the central lab) or an elevated ESR (defined as >the upper limit of normal [ULN] for the local lab) at screening were enrolled in the trial; patients must have been receiving MTX (oral or parenteral) at a dose of at least 10 mg/week for >6 months and at a stable dose and route of administration for >8 weeks prior to randomization (Day 0) and may have been receiving oral steroids, chronic NSAIDs and/or hydroxychloroquine.

The following are analyzed:

1. Proportion (%) of subjects achieving an ACR20 response in the tranilast 300 mg/day group compared to the placebo group at Week 12.

2. Proportion (%) of subjects achieving an ACR50 response in the tranilast 300 mg/day group compared to the placebo group at Week 12;

3. Proportion (%) of subjects achieving an ACR70 response in the tranilast 300 mg/day group compared to the placebo group at Week 12;

4. Proportion (%) of subjects achieving an ACR20 response in the tranilast 150 mg/day group compared to the placebo group at Week 12;

5. Proportion (%) of subjects achieving an ACR20 response in the combined tranilast (both dose groups) treatment groups compared to the placebo group at Week 12;

6. Analysis of dose response with tranilast utilizing the ACR20 and ACR50 response rates.

7. Mean change from baseline for each of the ACR components at Weeks 2, 4, 8, 12, and 16;

8. Proportion (%) of subjects achieving an ACR20 response at Weeks 2, 4, 8, 12, and 16;

9. Proportion (%) of subjects achieving an ACR50 response at Weeks 2, 4, 8, 12, and 16;

10. Proportion (%) of subjects achieving an ACR70 response at Weeks 2, 4, 8, 12, and 16;

11. Proportion (%) of subjects achieving a sustained ACR20 response, defined as an ACR20 response at Weeks 8 and 12;

12. Proportion (%) of subjects requiring rescue intervention;

13. Proportion (%) of subjects achieving low disease activity (score of ≦3.2) and/or remission (score of <2.6) as calculated by the Disease Activity Score 28 (DAS28) at Weeks 2, 4, 8, 12, and 16; and

14. EULAR responders, e.g. DAS28 “Good” or “Moderate” responders at Week 12.

The patents and publications listed herein are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described. It is recognized that various modifications are possible within the scope of the invention as claimed. It will also be understood that each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A method for treating an arthritic condition comprising administering to a subject in need thereof:
a. a therapeutically effective amount of tranilast or a pharmaceutical acceptable salt thereof; and
b. a therapeutically effective amount of a non-steroidal anti-inflammatory drug.

2. The method of claim 1 wherein said non-steroidal anti-inflammatory drug is ibuprofen, aspirin or naproxen.

3. The method of claim 1 wherein said therapeutically effective amount of tranilast or a pharmaceutical acceptable salt thereof and said therapeutically effective amount of an non-steroidal anti-inflammatory drug are comprised in a single pharmaceutical composition.

4. A method for treating an arthritic condition comprising administering to a subject in need thereof:
a. a therapeutically effective amount of tranilast or a pharmaceutical acceptable salt thereof; and
b. a therapeutically effective amount of a DMD selected from the group consisting of etanercept, adalimumab, infliximab, abatacept, an IL-1 receptor antagonist, a glucocorticoid, penicillamine, hydroxychloroquine sulfate, chlorambucil, cyclophosphamide, leflunomide, cyclosporine, auranofin, aurothioglucose, azathioprine, gold sodium thiomalate, methotrexate, cyclophosphamide, minocycline, sulfasalazine, rituximab, bucillamine, chloroquine, hydroxychloroquine, 6-mercaptopurine, lobenzarit, misoprostol, glucosamine and pharmaceutically acceptable salts thereof.

5. The method of claim 4 wherein said therapeutically effective amount of tranilast or a pharmaceutical acceptable salt thereof and said therapeutically effective amount of said DMD are comprised in a single pharmaceutical composition.

6. The method of claim 4 wherein said DMD is a TNF antagonist selected from the group consisting of etanercept, adalimumab and infliximab.

7. The method of claim 4 wherein said DMD is abatacept, rituximab, leflunomide, azathioprine, 6-mercaptopurine, chloroquine or hydroxychloroquine.

8. The method of claim 4 wherein said DMD is methotrexate.

9. The method of claim 4 wherein said DMD is a glucocorticoid selected from the group consisting of budesonide, prednisone and methylprednisolone.

10. The method of claim 4 wherein said DMD is a DMOAD selected from the group consisting of glucosamine, chondroitin sulfate, calcitonin, alendronate, risedronate, zoledronic acid, teriparatide, VX-765, pralnacasan, SB-462795, CPA-926, ONO-4817, S-3536, PG-530742, CP-544439 and pharmaceutically acceptable salts thereof.

11. A method for treating an arthritic condition comprising administering to a subject in need thereof:
a. a therapeutically effective amount of tranilast or a pharmaceutical acceptable salt thereof; and
b. a therapeutically effective amount of a selective COX-2 inhibitor, an antibiotic or an analgesic.

12. The method of claim 11 wherein said therapeutically effective amount of tranilast or a pharmaceutical acceptable salt thereof and said therapeutically effective amount of said selective COX-2 inhibitor, antibiotic or analgesic are comprised in a single pharmaceutical composition.

13. The method of claim 11 wherein said selective COX-2 inhibitor is selected from the group consisting of celecoxib, deracoxib, valdecoxib, parecoxib, rofecoxib, etoricoxib, lumiracoxib, PAC-10549, cimicoxib, GW-406381, LAS-34475, CS-502 and pharmaceutically acceptable salts thereof.

14. The method of claim 11 wherein said antibiotic is aminosalicylate, minocycline or doxycycline.

15. The method of claim 1 wherein said arthritic condition is rheumatoid arthritis, osteoarthritis or psoriatic arthritis.

16. The method of claim 4 wherein said arthritic condition is rheumatoid arthritis, osteoarthritis or psoriatic arthritis.

17. The method of claim 11 wherein said arthritic condition is rheumatoid arthritis osteoarthritis or psoriatic arthritis.

18. A pharmaceutical composition comprising
a. a therapeutically effective amount of tranilast or a pharmaceutical acceptable salt thereof; and
b. a therapeutically effective amount of a pharmaceutical agent selected from the group consisting of hydroxychloroquine, leflunomide, methotrexate, minocycline, cyclosporine, sulfasalazine, abatacept, adalimumab, entercept, infliximab, rituximab, anakinra, cyclophosphamide, penicillamine, tacrolimus, azathioprine, prednisone, methylprednisolone and pharmaceutically acceptable salts thereof.

19. The pharmaceutical composition of claim 18 wherein said amount of tranilast or salt thereof and said amount of said pharmaceutical agent together are an effective amount to treat rheumatoid arthritis.

20. The pharmaceutical of claim 18 wherein said pharmaceutical agent is methotrexate.
